SEQUENCE LISTING (28 sequences)

SEQ ID NO: 1 - DNA, 44 bp, Artificial
  Synthetic
gctttaatacgactcactatagggatgactgaatataaacttgt

SEQ ID NO: 2 - DNA, 21 bp, Artificial
  Synthetic
attgcactgtactcctcatga

SEQ ID NO: 3 - DNA, 44 bp, Artificial
  Synthetic
gctttaatacgactcactatagggtgaggagtacagtgcaatga

SEQ ID NO: 4 - DNA, 21 bp, Artificial
  Synthetic
ttgtgtctactgttctagaag

SEQ ID NO: 5 - DNA, 44 bp, Artificial
  Synthetic
gctttaatacgactcactataggggaacagtagacacaaaacag

SEQ ID NO: 6 - DNA, 21 bp, Artificial
  Synthetic
ttacataattacacactttgt

SEQ ID NO: 7 - DNA, 44 bp, Artificial
  Synthetic
gctttaatacgactcactatagggatacaatttgtacttttttc

SEQ ID NO: 8 - DNA, 21 bp, Artificial
  Synthetic
aaactctgggaatactggcac

SEQ ID NO: 9 - DNA, 44 bp, Artificial
  Synthetic
gctttaatacgactcactatagggaaatgcttattttaaaatga

SEQ ID NO: 10 - DNA, 20 bp, Artificial
  Synthetic
ttaatttgtttcacaccaac

SEQ ID NO: 11 - DNA, 44 bp, Artificial
  Synthetic
gctttaatacgactcactataggggtgcatgcagttgattactt

SEQ ID NO: 12 - DNA, 44 bp, Artificial; identical to SEQ ID NO: 11
  Synthetic

SEQ ID NO: 13 - RNA, 39 nt, Artificial
  Synthetic
cccguugcuuuuccucugggaaggauggcgcacgcuggg

SEQ ID NO: 14 - DNA, 39 bp, Artificial
  Synthetic
ccggaattcgccaccatgaacaagctttacatcggcaac

SEQ ID NO: 15 - DNA, 33 bp, Artificial
  Synthetic
ccgctcgagcttcctccgtgcctgggcctggtt

SEQ ID NO: 16 - DNA, 33 bp, Artificial
  Synthetic
ccgctcgagtcacttcctccgtgcctgggcctg

SEQ ID NO: 17 - DNA, 21 bp, Artificial
  Synthetic
acccaccttggcctcataaac

SEQ ID NO: 18 - DNA, 21 bp, Artificial
  Synthetic
actggcatctggtaggcactc

SEQ ID NO: 19 - DNA, 20 bp, Artificial
  Synthetic
gctgcttagacgctggattt

SEQ ID NO: 20 - DNA, 22 bp, Artificial
  Synthetic
gtcgaggtcatagttcctgttg

SEQ ID NO: 21 - DNA, 18 bp, Artificial
  Synthetic
ctctgcgggctgcttagt

SEQ ID NO: 22 - DNA, 24 bp, Artificial
  Synthetic
tttattcgaggttgaaaacagtga

SEQ ID NO: 23 - DNA, 22 bp, Artificial
  Synthetic
cctttcccatcaccaactacct

SEQ ID NO: 24 - DNA, 19 bp, Artificial
  Synthetic
gccgctgcctgtactcttc

SEQ ID NO: 25 - DNA, 23 bp, Artificial
  Synthetic
ggatttggaaagggtgtttattc

SEQ ID NO: 26 - DNA, 21 bp, Artificial
  Synthetic
tcccatctccttcatcacatc

SEQ ID NO: 27 - protein, 177 aa, Homo sapiens
VDIPLRLLVPTQYVGAIIGKEGATIRNITKQTQSKIDVHRKENAGAAEKAISVHSTPEGCSSACKMILEIMHKEAKDTKTADEVPLKILAHNNFVGRLIGKEGRNLKKVEQDTETKITISSLQDLTLYNPERTITVKGAIENCCRAEQEIMKKVIREAYENDVAAMSLQSHLIPGLN

SEQ ID NO: 28 - protein, 189 aa, Homo sapiens
PSSVTGAAPYSSFMQAPEQEMVQVFIPAQAVGAIIGKKGQHIKQLSRFASASIKIAPPETPDSKVRMVIITGPPEAQFKAQGRIYGKLKEENFFGPKEEVKLETHIRVPASAAGRVIGKGGKTVNELQNLTAAEVVVPRDQTPDEENDQVIVKIIGHFYASQMAQRKIRDILAQVKQQHQKGQSNQAQA

(19) United States
(12) Patent Application Publication
(10) Pub. No.: US 2024/0025886 A1
(43) Pub. Date: Jan. 25, 2024

(54) INHIBITORS OF IGF2BP1-RNA BINDING

(71) Applicants: YISSUM RESEARCH DEVELOPMENT COMPANY OF THE HEBREW UNIVERSITY OF JERUSALEM LTD., Jerusalem (IL); THE PENN STATE RESEARCH FOUNDATION, University Park, PA (US)

(72) Inventors: Vladimir SPIEGELMAN, Lancaster, PA (US); Vikash SINGH, Hershey, PA (US); Arun SHARMA, Hummelstown, PA (US); Amandeep SINGH, Hershey, PA (US); Joel YISRAELI, Efrat (IL); Nadav WALLIS, Ovnat (IL); Froma OBERMAN, Efrat (IL)

(73) Assignees: YISSUM RESEARCH DEVELOPMENT COMPANY OF THE HEBREW UNIVERSITY OF JERUSALEM LTD., Jerusalem (IL); THE PENN STATE RESEARCH FOUNDATION, University Park, PA (US)

(21) Appl. No.: 18/037,751

(22) PCT Filed: Nov. 18, 2021
(86) PCT No.: PCT/IL2021/051381

§ 371 Date: May 18, 2023

Related U.S. Application Data
(60) Provisional application No. 63/116,421, filed on Nov. 20, 2020.
(60) Provisional application No. 63/186,916, filed on May 11, 2021.

(51) Int. Cl. C07D 405/14; C07D 413/14; C07D 405/12; C07D 403/10; C07D 405/10; A61P 35/00

(52) U.S. Cl. CPC C07D 405/14; C07D 413/14; C07D 405/12; C07D 403/10; C07D 405/10; A61P 35/00

(57) ABSTRACT

The present invention is directed to compounds and compositions comprising thereof. Further, methods of use such as for the treatment and prevention of a disorder associated with binding of IGF2BP1 to an RNA in a subject in need thereof are also provided.

### CROSS REFERENCE TO RELATED APPLICATIONS

This application claims the benefit of priority to U.S. Provisional Patent Application No. 63/116,421 filed Nov. 20, 2020 entitled “INHIBITORS OF IGF2BP1-RNA BINDING”, and U.S. Provisional Patent Application No. 63/186,916 filed May 11, 2021, entitled “TARGETING THE RNA BINDING PROTEIN IGF2BP1 WITH A SMALL MOLECULE INHIBITOR” the contents of which are incorporated herein by reference in their entirety.

### FIELD OF THE INVENTION

The present invention relates to compounds for the treatment of medical disorders, and more particularly to inhibitors of IGF2BP1 binding to RNA useful for the treatment of cancers.

### BACKGROUND OF THE INVENTION

Kras is the most frequently mutated oncogene in humans and is a driver mutation in approximately 30% of lung adenocarcinomas. There are no effective targeted therapies for lung cancers driven by Kras-activating mutations. Although small molecule compounds targeting the most prominent Kras downstream mediators are available, they show minimal clinical efficacy in Kras mutant lung adenocarcinomas either as single agents or in combination with chemotherapy.

The insulin-like growth factor-II mRBA binding protein or IGF2BP (VICKZ) family of RNA binding proteins regulate RNA function at many levels, and one or more of the three IGF2BP paralogs is upregulated in many types of cancers. In lung adenocarcinoma patients, elevated IGF2BP1 expression correlates with lower overall survival, and this reduction is dramatically enhanced in patients with an oncogenic Kras gene. Further, IGF2BP1 not only binds Kras mRNA in lung adenocarcinoma cells but also synergizes with Kras to influence signaling and oncogenic activity.

There is a clear need for further therapeutics for the treatment of oncological disorders. This disclosure addresses this as well as other needs.

### SUMMARY OF THE INVENTION

The present disclosure provides compounds which are inhibitors of IGF2BP1 binding to RNA. Uses of these compounds in the treatment of medical disorders, for example cancer, are also provided.

In one aspect of the invention, there is provided a compound or a pharmaceutically acceptable salt thereof, wherein the compound is represented by Formula V:

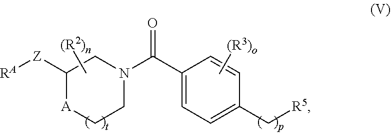

wherein: R^{A} is absent or selected from

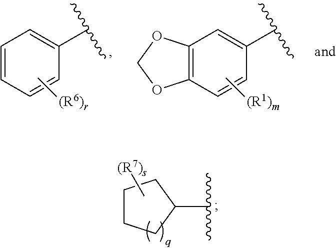

A is selected from CH and N—R, wherein R is

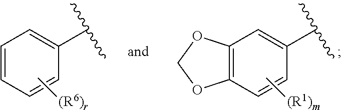

Z is absent, or selected from —C(O)— and —N(R⁸)—, wherein R⁸ is selected from hydrogen and C₁-C₆ alkyl; m is an integer ranging between 0 and 3; each n, o and s is independently an integer ranging between 0 and 4; p is an integer ranging between 0 and 8; q is an integer ranging between 0 and 2; r is an integer ranging between 0 and 5; t is an integer ranging between 1 and 3; R⁵ is selected from: a) 3- to 6-membered aliphatic ring, aromatic ring, or heteroaromatic ring having one or two ring heteroatoms independently selected from N, O, or S, wherein the 3- to 6-membered ring is optionally substituted with one or more X groups as allowed by valency; b) 5- to 10-membered monocyclic or bicyclic aliphatic ring, aromatic ring, or heteroaromatic ring having one, two, three, or four ring heteroatoms selected from N, O, or S, wherein the 5- to 10-membered ring is optionally substituted with one or more X groups as allowed by valency, wherein X is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃ alkyl), (5- to 10-membered monocyclic or bicyclic heteroaryl)(C₀-C₃ alkyl), R^{x}O—(C₀-C₃ alkyl)-, R^{x}S—(C₀-C₃ alkyl)-, (R^{x}R^{y}N)—(C₀-C₃ alkyl)-, RzC(O)—O—(C₀-C₃ alkyl)-, RzC(O)—(RxN)—(C₀-C₃ alkyl)-, R^{z}S(O)₂—O—(C₀-C₃ alkyl)-, R^{z}S(O)₂—(R^{x}N)—(C₀-C₃ alkyl)-, RzC(O)—, R^{z}S(O)—, and R^{z}S(O)₂—, each of which may be optionally substituted with one or more Y groups as allowed by valency, wherein Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol; c) —SeR⁹, wherein R⁹ is selected from hydrogen, cyano, alkyl, cyloalkyl, heterocycle, aryl, or heteroaryl, each of which R⁹ may be optionally substituted with one or more X groups as allowed by valency; and d) —NH(C═W)NH₂, wherein W is S or Se; each X, R¹, R², R³, R⁶ and R⁷ represents one or more substituents, each substituent is independently selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency, or wherein two or more of the substituents are interconnected to form a fused aromatic ring, a fused aliphatic ring, or a fused heteroaromatic ring; each R^{x} and R^{y} is independently selected from hydrogen, C₁-C₆alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is selected from hydrogen, halo, C₁-C₆ alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol; and if A is N—R, then t is 1 and Z and R^{A} are absent; and if A is CH, Z is NH, p is 1, and R^{A} is

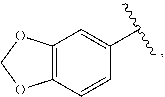 [SMILES: *c1ccc2c(c1)OCO2]

then R⁵ is devoid of an unsubstituted tetrazole.

In some embodiments, the compound is represented by Formula VIa:

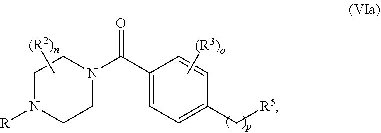

### or Formula VIb:

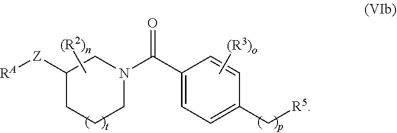

In some embodiments, r is an integer ranging between 1 and 5.

In some embodiments, R^{A} is

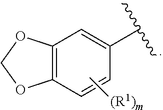

In some embodiments, R is selected from and

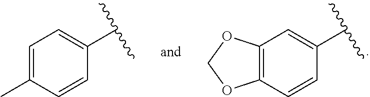

In some embodiments, R^{A} is

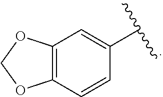 [SMILES: *c1ccc2c(c1)OCO2]

In some embodiments the compound is represented by Formula VIIIa:

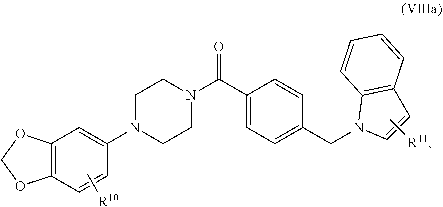

wherein: each R¹⁰ and R¹¹ is independently selected from hydrogen, halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, RzC(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; and Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

In some embodiments, the compound is represented by formula VIIIb:

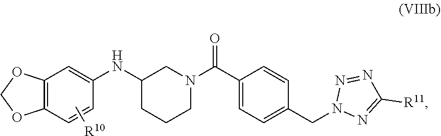

wherein: each R¹⁰ and R¹¹ is selected from hydrogen, halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl), R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol; and if R¹⁰ is hydrogen then R¹¹ is not hydrogen.

In some embodiments, R⁵ is selected from:

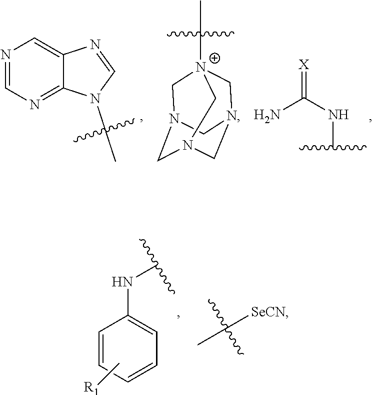

wherein: X is Se, O or S; and R₁ is selected from H, F, Cl, Br, NO₂, NH₂, CH₃, C₂H₅, CF₃, OH, CN, C₆H₅, CHO, COOH, and any combination thereof.

In some embodiments, R⁵ is selected from:

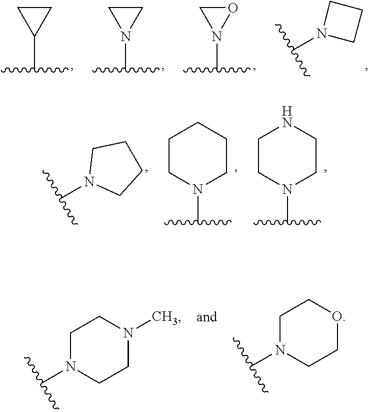

In some embodiments, R⁵ is selected from:

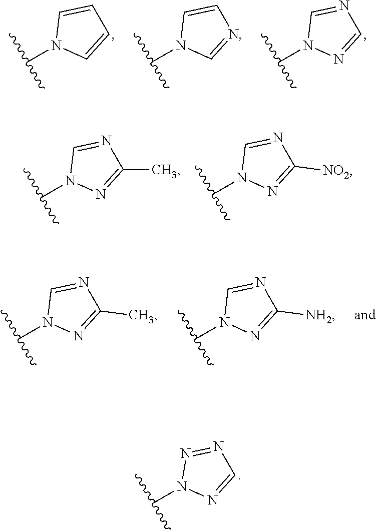

In some embodiments, R⁵ is selected from:

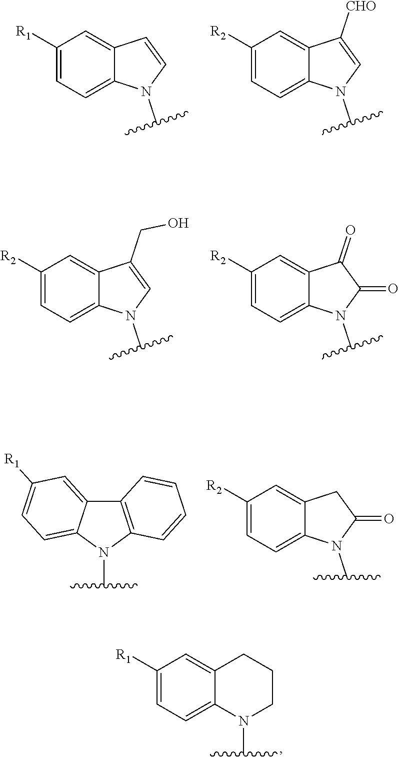

wherein: each R₁ and R₂ is independently selected from H, F, Cl, Br, NO₂, NH₂, CH₃, C₂H₅, CF₃, OH, CN, C₆H₅, CHO, COOH, and any combination thereof.

In some embodiments, R⁵ is selected from:

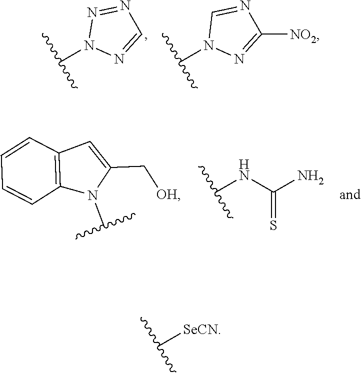

In some embodiments, Z is —N(R⁸)—.

In some embodiments, R⁸ is hydrogen.

In some embodiments, p is 1.

In some embodiments, the compound comprises:

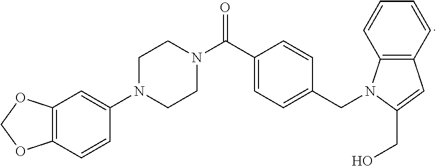 [SMILES: O=C(c1ccc(Cn2c(CO)cc3ccccc32)cc1)N1CCN(c2ccc3c(c2)OCO3)CC1]

In another aspect of the invention, there is provided a pharmaceutical composition comprising a therapeutically effective amount of a compound of the present invention, or a pharmaceutically acceptable salt thereof, and a pharmaceutically acceptable carrier or excipient, wherein: R^{A} is absent or selected from

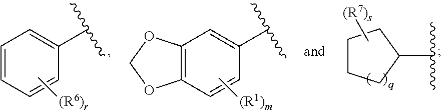

A is selected from CH and N—R, wherein R is

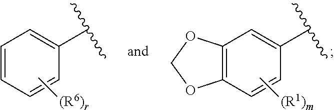

Z is absent, or selected from —C(O)— and —N(R⁸)—, wherein R⁸ is selected from hydrogen and C₁-C₆ alkyl; m is an integer ranging between 0 and 3; each n, o and s is independently an integer ranging between 0 and 4; p is an integer ranging between 0 and 8; q is an integer ranging between 0 and 2; r is an integer ranging between 0 and 5; t is an integer ranging between 1 and 3; R⁵ is selected from: a) 3- to 6-membered aliphatic ring, aromatic ring, or heteroaromatic ring having one or two ring heteroatoms independently selected from N, O, or S, wherein the 3- to 6-membered ring is optionally substituted with one or more X groups as allowed by valency; b) 5- to 10-membered monocyclic or bicyclic aliphatic ring, aromatic ring, or heteroaromatic ring having one, two, three, or four ring heteroatoms selected from N, O, or S, wherein the 5- to 10-membered ring is optionally substituted with one or more X groups as allowed by valency, wherein X is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃ alkyl), (5- to 10-membered monocyclic or bicyclic heteroaryl)(C₀-C₃ alkyl), R^{x}O—(C₀-C₃ alkyl)-, RxS—(C₀-C₃ alkyl)-, (RxRyN)—(C₀-C₃ alkyl)-, RzC(O)—O—(C₀-C₃ alkyl)-, RzC(O)—(RxN)—(C₀-C₃ alkyl)-, RzS(O)₂—O—(C₀-C₃ alkyl)-, RzS(O)₂—(RxN)—(C₀-C₃ alkyl)-, RzC(O)—, RzS(O)—, and R^{z}S(O)₂—, each of which may be optionally substituted with one or more Y groups as allowed by valency, wherein Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol; c) —SeR⁹, wherein R⁹ is selected from hydrogen, cyano, alkyl, cyloalkyl, heterocycle, aryl, or heteroaryl, each of which R⁹ may be optionally substituted with one or more X groups as allowed by valency; and d) —NH(C═W)NH₂, wherein W is S or Se; each X, R¹, R², R³, R⁶ and R⁷ represents one or more substituents, each substituent is independently selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency, or wherein two or more of the substituents are interconnected to form a fused aromatic ring, a fused aliphatic ring, or a fused heteroaromatic ring; each R^{x} and R^{y} is independently selected from hydrogen, C₁-C₆alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is selected from hydrogen, halo, C₁-C₆ alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

In some embodiments, the therapeutically effective amount comprises a concentration of the compound within the pharmaceutical composition of between 100 nM and 50 μM.

In some embodiments, the pharmaceutical composition is for use in the inhibition of insulin like growth factor 2 mRNA binding protein 1 (IGF2BP1) activity.

In some embodiments, the pharmaceutical composition is for use in the prevention or treatment of a disorder associated with cancer in a subject in need thereof.

In another aspect of the invention, there is provided a method for preventing or treating a disease or a disorder associated with binding of IGF2BP1 to an RNA in a subject in need thereof, comprising administering to the subject a therapeutically effective amount of the compound of the present invention, or the pharmaceutical composition of the present invention, thereby preventing or treating a disease or a disorder associated with binding of IGF2BP1 to an RNA in the subject.

In some embodiments, the disease comprises a cell proliferation related disease.

In some embodiments, the cell proliferation related disease comprises cancer.

In some embodiments, the cancer comprises any one of a metastatic cancer, a solid tumor, and a liquid tumor.

In another aspect of the invention, there is provided a method for treating or preventing cancer in a subject, comprising administering to the subject a therapeutically effective amount of the compound of the present invention, or the pharmaceutical composition of the present invention, thereby treating or preventing cancer in the subject.

In some embodiments, the subject is a human subject.

In some embodiments, the administering comprises an administration route selected from intravenous administration, intraperitoneal administration, subcutaneous administration, or any combination thereof.

In some embodiments, the treating comprises: (i) reducing intracellular expression of at least one protooncogene, (ii) preventing or reducing metastasis, or both (i) and (ii).

In some embodiments, the method further comprises a step preceding the administering, comprising determining abundance or levels of any one of: IGF2BP1 transcripts or a protein product thereof, IGF2BP1-RNA complexes, or both, in the subject, wherein an increase in any one of the IGF2BP1 transcripts or a protein product thereof, the IGF2BP1-RNA complexes, or both, in the subject compared to a control, is indicative of the subject being suitable for the treating.

In some embodiments, the determining is in a sample obtained or derived from the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-D Present the identification of a fragment of Kras mRNA to probe for Igf2bp1 binding. Bar graph presenting different fragments spanning the Kras mRNA coding region, and part of its 3′UTR, were 5′ end labeled with fluorescein and used as substrates for Igf2bp1 binding in an fluorescence polarization (FP) assay. Two fragments from cofilin mRNA, cfl7 and cfl4, that were previously shown to bind or not bind, respectively, Igf2bp1, were included as controls. (Y axis, FP ratio; no shift—i.e., no binding—FP ratio=1.) (FIG. 1A); schematic map of the different fragments of Kras used in the FP assay of FIG. 1A (FIG. 1B); increasing concentrations of Igf2bp1 protein (indicated at the top of the lanes), incubated with 100 nM fluorescently labeled Kras 6 RNA, retarded migration of the RNA in a non-denaturing polyacrylamide gel (EMSA) (FIG. 1C); and a graph presenting the kinetics of Kras 6 RNA binding to Igf2bp1 monitored by microScale thermophoresis (MST); insert, a representative MST thermophoresis curve (FIG. 1D);

FIGS. 2A-E present the high throughput screening for Igf2bp1 inhibitors: a snake plot of the FP screen for inhibitors of Igf2bp1 binding to fluorescent Kras 6 RNA (FIG. 2A). Over 27,000 compounds were screened (X-axis), and the robust Z score for each assay was plotted along the Y-axis. The circle indicates the position of compound 7773; chemical structure of compounds 7773 and 393 (FIG. 2B); graphs of activity of protein-RNA binding of compounds 7773 and 393 (FIG. 2C). 7773 or 393 was added at increasing concentrations (X-axis), and the percent activity of protein-RNA binding was plotted on the Y-axis. For Igf2bp1 binding to Kras 6 RNA, the IC₅₀ for 7773 ranged from 15-36 μM (n=4), and for 393, the IC₅₀ was greater than 100 μM (n=1). As a control, the ability of 7773 to inhibit binding of a different RNA binding protein, La, to Bcl2 RNA was assayed, and its IC₅₀ was much greater than 100 μM (n=3); MST was used to determine the Kd of Igf2bp1 binding to either 7773 or 393 (FIG. 2D); and a graph showing no binding of 7773 to La in an MST assay (FIG. 2E); Representative MST thermophoresis curves are inserted in FIG. 2D and FIG. 2E;

FIGS. 3A-C present the binding of 7773 to RNA binding domains of Igf2bp1: domain structure organization of Igf2bp1 (FIG. 3A). Six RNA binding domains are organized in three di-domains: RRM12, KH12 and KH34. The arrowheads indicate the boundaries of the individual RNA binding domains; the di-domain constructs used in this study are indicated by arrows below the map; MST binding curves of 7773 to the di-domains RRM12, KH12, and KH34 (FIG. 3B). Representative MST thermophoresis curves are inserted. Overlay of ¹⁵N-HSQC spectra recorded on the free RRM12, KH12 and KH34 di-domains and at a 1:14 protein-to-7773 molar ratio (FIG. 3C). The dispersed region between ¹H 8.3-9.6 and ¹⁵N 111.6-127.2 ppm is displayed, as representative of the full spectra of the titration reported in FIG. 13. Grey arrowheads indicate peaks that shifted significantly upon addition of 7773;

FIGS. 4A-H present 7773 binding surface: KH34 per-residue chemical shift perturbations (CSP) recorded upon 1:14 7773 binding (FIG. 4A). The boxes indicate the KH3 and KH4 domains boundaries. The dotted lines indicate, respectively, one, two and three standard deviations of the average CSP observed; model representation of KH34 di-domain (FIG. 4B); model of the binding surface of 7773 on KH34 (FIG. 4C). The left panel is oriented as in FIG. 4B, and the right panel with a 180° rotation; model of DALI structural alignment of Igf2bp1 KH12 and KH34 di-domains (FIG. 4D); primary sequence CLUSTAL X alignment of KH12 and KH34 (FIG. 4E). The secondary structure elements are reported above and below KH12 and KH34 sequences, respectively. The residues of the binding surface are indicated by arrowheads; model of KH34 hydrophobicity (FIG. 4F). The panel is oriented as in FIG. 4B; overlay of the ¹⁵N-HSQC spectra of KH34 in the apo and complex with 7773 in low and high salt conditions (FIG. 4G). The panel reports a zoom in the representative region between ¹H 8.66-9.10 and ¹⁵N 111.50-116.00 ppm; and a bar graph of KH34 per-residue chemical shift perturbations (CSP) recorded upon 1:14 7773 binding in low and high salt (FIG. 4H);

FIGS. 5A-C present the inhibition of Igf2bp1 RNA binding in vitro: EMSA with fluorescent Kras 6 RNA comparing the effect of incubation of increasing concentrations of Igf2bp1 with either DMSO or 200 mM 7773 on retardation of RNA migration (FIG. 5A); EMSA with fluorescent Bcl2 RNA comparing the effect of incubation of increasing concentrations of La protein with either DMSO or 200 mM 7773 on retardation of RNA migration (FIG. 5B) and MST analysis of the effect of 7773 on the binding of Igf2bp1 to Kras 6 RNA (FIG. 5C). In the absence of compound, the K_{D}=56 nM; in the presence of 50 mM 7773, the K_{D}=63 nM, and in the presence of 100 mM 7773, the K_{D}=120 nM. Representative MST thermophoresis curves are inserted;

FIGS. 6A-C present the binding of 7773 to other Igf2 bp paralogues: MST analysis of 7773 incubation with Igf2bp2 (FIG. 6A) and Igf2bp3 (FIG. 6B). Representative MST thermophoresis curves are inserted. EMSA with fluorescent Kras 6 RNA comparing the effect of incubation of increasing concentrations of Igf2bp3 with either DMSO or 200 mM 7773 on retardation of RNA migration (FIG. 6C). *, indicates that some of the sample leaked out of the lane;

FIG. 7 presents a graph showing 7773 targets human Igf2bp1 in transfected mouse LKR-M cells. LKR-M cells were transfected with either full length human Igf2bp1-GFP (FI, circles) or GFP alone (GFP, triangles) and then assayed by Incucyte Live Cell Analysis System for their ability to migrate in a wound healing assay in the presence of either 20 mM 7773 or DMSO;

FIGS. 8A-E present the use of a split-luciferase assay to observe 7773 targeting of Igf2bp1 in transfected RKO cells: schematic presentation of assay principal: luciferase activity is detected only when Igf2bp1 dimerizes on target RNA (FIG. 8A); four split luciferase reporter plasmids fused with Igf2bp1 in different orientations vis-à-vis the luciferase fragments (left) were assessed for efficiency of luciferase activity in RKO cells (right) (FIG. 8B); ectopic expression of wild-type Igf2bp1 inhibits split-luciferase activity (FIG. 8C). Treatment with 50 mM 7773 inhibited split-luciferase activity (FIG. 8D), but not control luciferase in RKO cells (FIG. 8E);

FIGS. 9A-E present that 7773 downregulates Kras and other Igf2bp1 target RNAs, Kras protein, and downstream signaling: Real-time PCR analysis of steady state levels of CD44, Kras, cMyc, and SRF RNA in ES2 or H1299 cells treated with either 20 mM 7773 or DMSO for 12 or 24 hours (FIG. 9A); western blot analysis of Kras expression in H1299 cells incubated in 20 mM 7773, 10 mM 7773, or DMSO for 48 hours (FIG. 9B). Duplicates were performed and analyzed for each concentration. Tubulin was used as loading control; bar graph of the quantification of the blot in FIG. 9B, normalized to tubulin and to the relative level of Kras protein in the DMSO-treated samples (FIG. 9C); H1299 cells were cultured with either 20 mM 7773 or DMSO for 24 hours, after which FCS was removed from the medium and the cells were cultured for another 24 hours (FIG. 9D). At that point, FCS was re-added for either 10′ or 30′ and then protein extracts were prepared and analyzed by western blot for expression of ERK and phosphoERK. Cells grown continuously in FCS for 48.5 hours were used as controls. Experimental duplicates were run on the gel; and bar graph of the quantification of the western shown in FIG. 9D, with the ratio of pERK to ERK plotted on the Y-axis; Quantification of the western shown in (D), with the ratio of pERK to ERK plotted on the Y-axis (FIG. 9E).

FIGS. 10A-D present that 7773 inhibits wound healing and growth in soft agar without affecting cell proliferation: graph of H1299, ES2, and HEK293 cells incubated with DMSO or different concentrations of 7773 (5 mM; 10 mM; 20 mM) and analyzed for wound healing (FIG. 10A) or cell proliferation using the Incucyte Live Cell Analysis System (FIG. 10B); picture of H1299 cells seeded in triplicate in soft agar in the presence of either DMSO or 7773 (20 mM) and cultured for 2 weeks (FIG. 10C) and bar graph of the quantification of the results presented in FIG. 10C; FIG. 10D is a bar graph representing quantification of the results in FIG. 10C.

FIGS. 11A-B present Kras 6 RNA competing better for binding than Kras 2: picture of EMSA assay assessing the ability of unlabeled Kras 2 and Kras 6 RNA to compete for Igf2bp1 binding to fluorescent Kras 6 RNA (FIG. 11A); the fold excess of unlabeled RNA is shown at the top of each lane. Bar graph showing the unlabeled Kras 6 RNA, when included in excess in the FP reaction, reduces the FP shift by competing for Igf2bp1 binding (FIG. 11B);

FIG. 12 is a graph showing that Kras 6 RNA binds to KH34 but not to KH12 or RRM12. MST was used to test for the ability of Kras 6 RNA to bind to each of the di-domains of Igf2bp1. Representative MST thermophoresis curves are inserted.

FIG. 13 is a graph showing that 7773 does not inhibit G9a enzymatic activity: 7773 was incubated at increasing concentrations (X-axis) in a fluorescence polarization G9a enzymatic activity assay (see Materials and Methods). 100% activity indicates low fluorescence polarization (i.e., no inhibition);

FIG. 14 presents a ¹⁵N-HSQC spectra of KH34: Overlay of the ¹⁵N-HSQC spectra of KH34 in the apo and holo form with 7773. The figure reports the assignment of the residues that showed CSP values above at least one standard deviation of the average CSP observed. The shifting peaks are indicated by a black cross;

FIG. 15 presents a plot reporting the overlay of the ¹⁵N-HSQC spectrum of apo-KH3 with the spectra obtained upon addition of 1:1, 1:2, 1:4, 1:8, 1:14 protein-to-7773 molar ratios in 150 mM NaCl conditions. The addition of increasing amounts of 7773 induced greater shifts when compared with the same titration performed with 50 mM NaCl, suggesting that the binding of 7773 to KH34 is hydrophobically driven. The blow-up reports the CSP variation for the reporter G472 in titration with 7773 in low and high salt conditions;

FIG. 16 is a picture of EMSA with fluorescent Kras 6 RNA comparing the effect of incubation of increasing concentrations of Igf2bp1 with either DMSO or 200 mM 393 on retardation of RNA migration;

FIGS. 17A-B are bar graphs showing the control RNAs are not affected by incubation with 7773: real-time PCR analysis of steady state levels of 185, emc7, or RPLPO RNAs (all RNAs that are not targets of Igf2bp1) in ES2 (FIG. 17A) or H₁₂₉₉ (FIG. 17B) cells treated with either 20 mM 7773 or DMSO for 24 hours;

FIG. 18 is a bar graphs showing 7773 inhibits signaling in LKR-M-Fl cells: LKR-M cells transfected with full length human Igf2bp1 were treated with 7773 as described in FIG. 9D. The ratio of pERK/ERK was quantified from a western blot and plotted, normalized to the DMSO control;

FIGS. 19A-B are bar graphs showing the 7773 does not affect cytotoxicity or cell viability: H1299 cells were incubated with DMSO or 20, 10, or 5 mM 7773 for 48 hours and tested for cytotoxicity (FIG. 19A) or cell viability (FIG. 19B); and

FIGS. 20A-B present graphs comparing compound 7773 and analog compound 15 targeting human Igf2bp1 in transfected mouse LKR-M cells. LKR-M cells were transfected with either full length human Igf2bp1-GFP (Fl, circles) or GFP alone (GFP, triangles) and then assayed by Incucyte Live Cell Analysis System for their ability to migrate in a wound healing assay in the presence of either 20 μM 7773 or DMSO (FIG. 20A) or 10 μM compound 15 or DMSO (FIG. 20B).

### DETAILED DESCRIPTION OF THE INVENTION

According to some embodiments, the present invention provides a compound represented Formula I to Formula VIII as described herein.

According to some embodiments, the present invention provides a compound represented by Formula V as described herein.

In some embodiments, provided herein is a composition comprising a compound of Formula V and a carrier. In some embodiments, provided herein is a pharmaceutically acceptable salt of a compound of the invention such as but not limited to the compound of Formula V. In some embodiments, the composition comprises the compound of the invention, a pharmaceutically acceptable salt thereof or both. In some embodiments, the composition is a pharmaceutical composition, comprising the compound of the invention (and/or any pharmaceutically acceptable salt, or any derivative thereof) and a pharmaceutically acceptable carrier.

In some embodiments, the compound or the composition of the present invention inhibits insulin like growth factor 2 mRNA binding protein 1 (IGF2BP1) activity.

According to some embodiments, the present invention provides a compound or a composition (e.g. a pharmaceutical composition) as described herein, for use in the prevention or treatment of a disorder associated with cancer.

According to some embodiments, the present invention provides a method for preventing or treating a disease or a disorder associated with binding of IGF2BP1 to an RNA in a subject in need thereof.

### Compounds

According to some embodiments, the present invention provides a compound represented by Formula V:

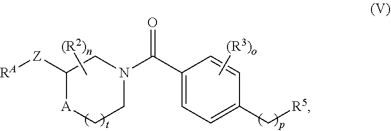

wherein: R^{A} is absent or selected from

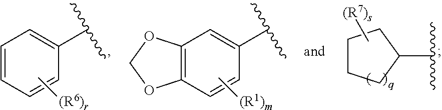

A is selected from CH and N—R, wherein R is

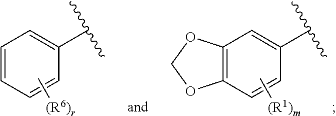

Z is absent, or selected from —C(O)— and —N(R⁸)—, wherein R⁸ is selected from hydrogen and C₁-C₆ alkyl; m is an integer ranging between 0 and 3; each n, o and s is independently an integer ranging between 0 and 4; p is an integer ranging between 0 and 8; q is an integer ranging between 0 and 2; r is an integer ranging between 0 and 5; t is an integer ranging between 1 and 3; R⁵ is selected from: a) 3- to 6-membered aliphatic ring, aromatic ring, or heteroaromatic ring having one or two ring heteroatoms independently selected from N, O, or S, wherein the 3- to 6-membered ring is optionally substituted with one or more X groups as allowed by valency; b) 5- to 10-membered monocyclic or bicyclic aliphatic ring, aromatic ring, or heteroaromatic ring having one, two, three, or four ring heteroatoms selected from N, O, or S, wherein the 5- to 10-membered ring is optionally substituted with one or more X groups as allowed by valency, wherein X is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃ alkyl), (5- to 10-membered monocyclic or bicyclic heteroaryl)(C₀-C₃ alkyl), R^{x}O—(C₀-C₃ alkyl)-, RxS—(C₀-C₃ alkyl)-, (RxRyN)—(C₀-C₃ alkyl)-, R^{z}C(O)—O—(C₀-C₃ alkyl)-, RzC(O)—(RxN)—(C₀-C₃ alkyl)-, R^{z}S(O)₂—O—(C₀-C₃ alkyl)-, R^{z}S(O)₂—(R^{x}N)—(C₀-C₃ alkyl)-, RzC(O)—, RzS(O)—, and R^{z}S(O)₂—, each of which may be optionally substituted with one or more Y groups as allowed by valency, wherein Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol; c) —SeR⁹, wherein R⁹ is selected from hydrogen, cyano, alkyl, cyloalkyl, heterocycle, aryl, or heteroaryl, each of which R⁹ may be optionally substituted with one or more X groups as allowed by valency; and d) —NH(C═W)NH₂, wherein W is S or Se; each X, R¹, R², R³, R⁶ and R⁷ represents one or more substituents, each substituent is is independently selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-R^{x}S—(C0-C3 alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency, or wherein two or more of the substituents are interconnected to form a fused aromatic ring, a fused aliphatic ring, or a fused heteroaromatic ring; each R^{x} and R^{y} is independently selected from hydrogen, C₁-C₆alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is selected from hydrogen, halo, C₁-C₆ alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol. In some embodiments, if A is N—R, then t is 1, and Z and R^{A} are absent. In some embodiments, if A is CH, Z is NH, p is 1, and R^{A} is

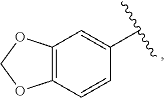 [SMILES: *c1ccc2c(c1)OCO2]

then R⁵ is devoid of an unsubstituted tetrazole. In some embodiments, R⁵ is devoid of alkyl chain or substituted alkyl chain.

In some embodiments, m is an integer ranging between 0 and 2. In some embodiments, m is 0, 1, 2 or 3. Each possibility represents a separate embodiment of the invention. In some embodiments, each n, o and s is independently an integer ranging between 0 and 4, 0 and 3, 0 or 0 and 2. In some embodiments, each n, o and s is 0, 1, 2, 3 or 4. Each possibility represents a separate embodiment of the invention. In some embodiments, p is an integer ranging between 0 and 8, between 0 and 7, between 0 and 5, between 0 and 3, or between 0 and 2, including any range therebetween. In some embodiments, p is 0, 1, 2, 3, 4, 5, 6, 7 or 8. Each possibility represents a separate embodiment of the invention. In some embodiments, q is 0, 1, or 2. In some embodiments, r is 0, 1, 2, 3, 4 or 5. In some embodiments, t is 1, 2 or 3. Each possibility represents a separate embodiment of the invention.

In some embodiments, the compound is represented by Formula VIa:

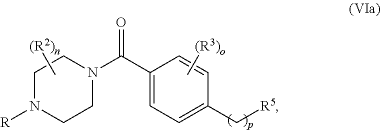

wherein R, R², R³, R⁵, n, o and p are as described herein.

In some embodiments, R⁵ is

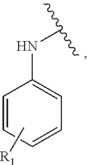

wherein R₁ is selected from H, F, Cl, Br, NO₂, NH₂, CH₃, C₂H₅, CF₃, OH, CN, C₆H₅, CHO, COOH, and any combination thereof.

In some embodiments, R⁵ is selected from:

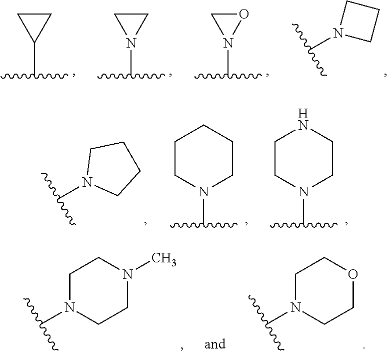

In some embodiments, R⁵ is a 5- to 10-membered monocyclic or bicyclic heteroaryl having one, two, three, or four ring heteroatoms selected from N, O, and S, and optionally substituted with one or more (e.g., one, two, three, or four) X groups.

In some embodiments, R⁵ is a 5-membered monocyclic heteroaryl having one two, three, or four ring heteroatoms selected from N, O, and S, and optionally substituted with one or more (e.g., one, two, three, or four) X groups.

In some embodiments, R⁵ is pyrrolyl, imidazolyl, pyrazolyl, triazolyl, or tetrazolyl, each of which is optionally substituted with one, two, three, or four X groups.

In some embodiments, R⁵ is selected from:

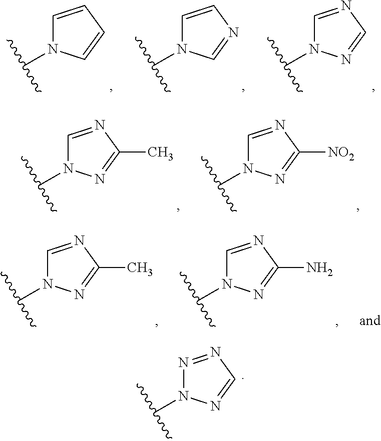

In some embodiments, R⁵ is a 9- to 10-membered bicyclic heteroaryl having one, two, three or four ring heteroatoms selected from N, O, and S, and optionally substituted with one or more (e.g., one, two, three, or four) X groups.

In some embodiments, R⁵ is 1-indolyl optionally substituted with one, two, three, or four X groups.

In some embodiments, R⁵ is selected from

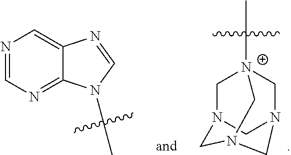 [SMILES: *n1cnc2cncnc21 ; *[N+]12CN3CN(CN(C3)C1)C2]

In some embodiments, R⁵ is

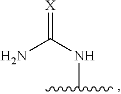

wherein X is Se, O or S.

In some embodiments, R⁵ is selected from:

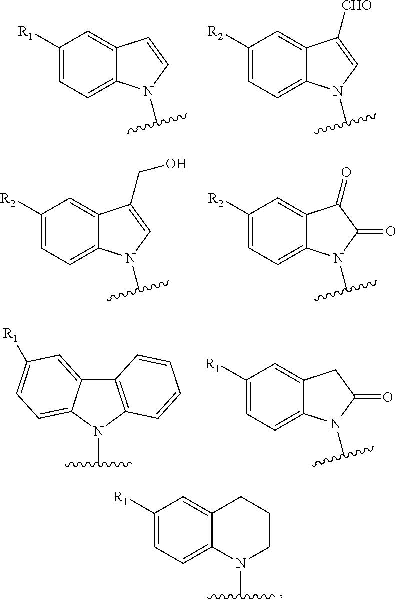

wherein each R₁ and R₂ is independently selected from H, F, Cl, Br, NO₂, NH₂, CH₃, C₂H₅, CF₃, OH, CN, C₆H₅, CHO, COOH, and any combination thereof.

In some embodiments, R⁵ is selected from:

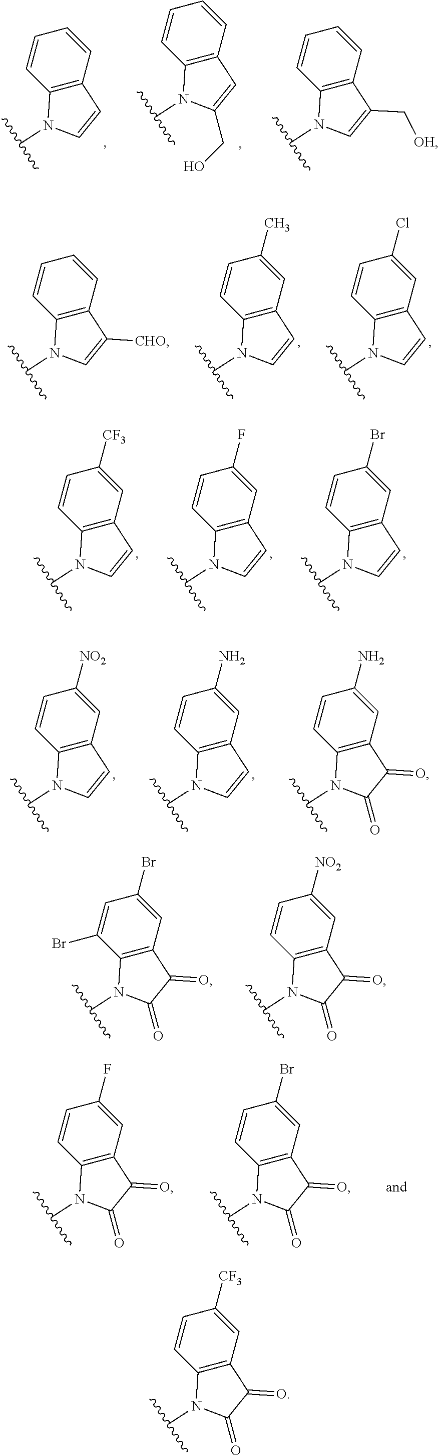

In some embodiments, R⁵ is —SeCN. In some embodiments, R⁵ is —NHC(═S)NH₂.

In some embodiments, R⁵ is selected from

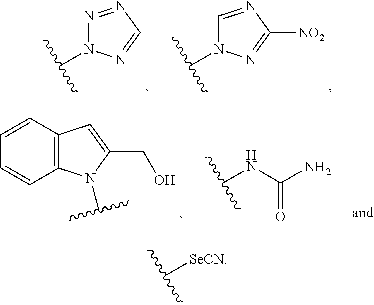

In some embodiments, the compound is represented by Formula VIIa:

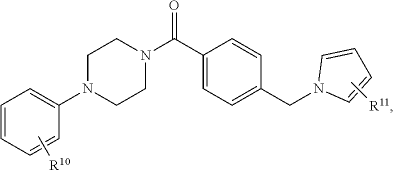

wherein R¹⁰ is selected from hydrogen, halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; R¹¹ is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; R¹¹ is selected from, halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C0-C3 alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; R¹¹ is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; and Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

In some embodiments, the compound is represented by Formula VIIIa:

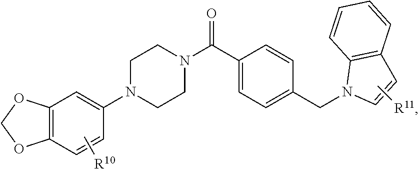

wherein each R¹⁰ and R¹¹ is independently selected from hydrogen, halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; and Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

In some embodiments, the compound comprises any one of:

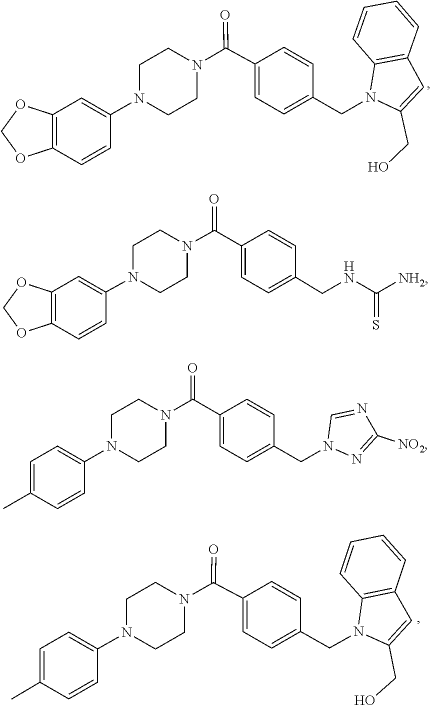 [SMILES: O=C(c1ccc(Cn2c(CO)cc3ccccc32)cc1)N1CCN(c2ccc3c(c2)OCO3)CC1 ; NC(=S)NCc1ccc(C(=O)N2CCN(c3ccc4c(c3)OCO4)CC2)cc1 ; Cc1ccc(N2CCN(C(=O)c3ccc(Cn4cnc([N+](=O)[O-])n4)cc3)CC2)cc1 ; Cc1ccc(N2CCN(C(=O)c3ccc(Cn4c(CO)cc5ccccc54)cc3)CC2)cc1]

combination thereof.

In some embodiments, the compound is selected from the group consisting of:

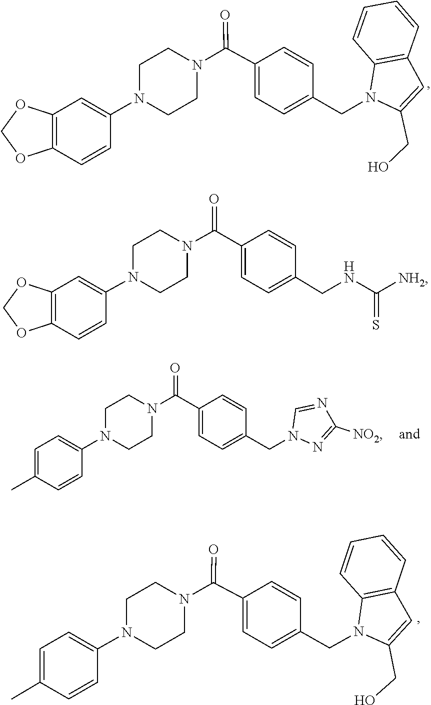 [SMILES: O=C(c1ccc(Cn2c(CO)cc3ccccc32)cc1)N1CCN(c2ccc3c(c2)OCO3)CC1 ; NC(=S)NCc1ccc(C(=O)N2CCN(c3ccc4c(c3)OCO4)CC2)cc1 ; Cc1ccc(N2CCN(C(=O)c3ccc(Cn4cnc([N+](=O)[O-])n4)cc3)CC2)cc1 ; Cc1ccc(N2CCN(C(=O)c3ccc(Cn4c(CO)cc5ccccc54)cc3)CC2)cc1]

In some embodiments, the compound is represented by Formula VIb:

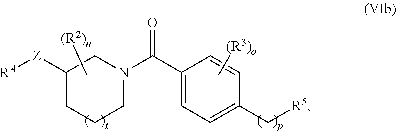

wherein Z, R^{A}, R², R³, R⁵, t, n, o and p are as described herein.

In some embodiments, the compound is represented by Formula VIIb:

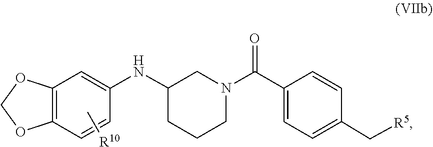

wherein R⁵ is as described hereinabove, and R¹⁰ is selected from hydrogen, halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; R¹¹ is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-(5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; and Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

In some embodiments, the compound is represented by formula VIIIb:

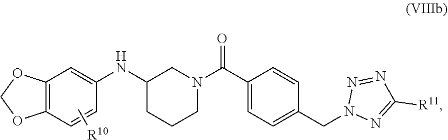

wherein R¹⁰ is as described hereinabove, and R¹¹ is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; R¹¹ is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; and Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

In some embodiments, the compound comprises any one of:

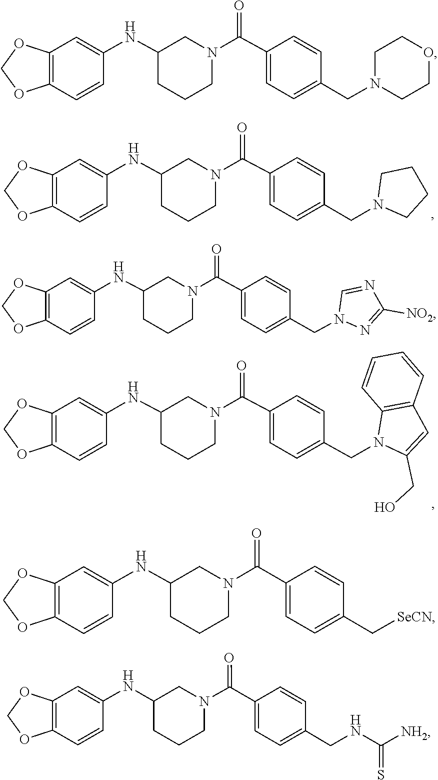

or any combination thereof.

In some embodiments, the compound is selected from the group consisting of:

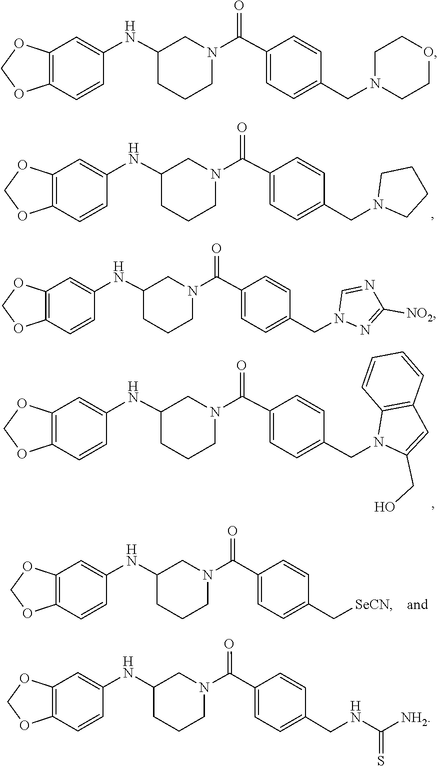

According to some embodiments, the present invention provides a compound represented by Formula I:

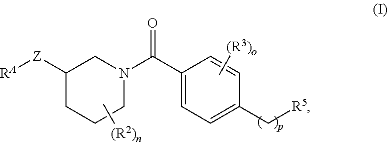

or a pharmaceutically acceptable salt thereof, wherein:
R^{A} is absent or selected from

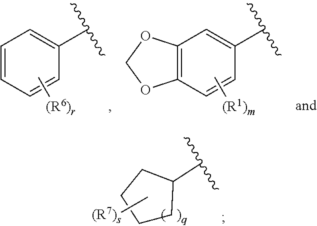

Z is absent or selected from —C(O)— and —N(R⁸)—; n is 0, 1, 2, 3, or 4; o is 0, 1, 2, 3, or 4; p is 0, 1, 2, 3, 4, 5, 6, 7, or 8; q is 0, 1, or 2; r is 0, 1, 2, 3, 4, or 5; s is 0, 1, 2, 3, or 4; R² and R³ are independently selected at each occurrence from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z} S(O)₂—, each of which may be optionally substituted with one or more Y groups as allowed by valency; R⁵ is selected from: a) 3- to 6-membered aliphatic ring, aromatic ring, or heteroaromatic ring having one or two ring heteroatoms independently selected from N, O, or S, wherein the 3- to 6-membered ring is optionally substituted with one or more X groups as allowed by valency; b) 5- to 10-membered monocyclic or bicyclic aliphatic ring, aromatic ring, or heteroaromatic ring having one, two, three, or four ring heteroatoms selected from N, O, or S, wherein the 5- to 10-membered ring is optionally substituted with one or more X groups as allowed by valency; c) —SeR⁹, wherein R⁹ is selected from hydrogen, cyano, alkyl, cyloalkyl, heterocycle, aryl, or heteroaryl, each of which R⁹ may be optionally substituted with one or more X groups as allowed by valency; and d) —NH(C═W)NH₂, wherein W is S or Se; R⁶ and R⁷ are independently selected at each occurrence from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃ alkyl), (5- to 10-membered monocyclic or bicyclic heteroaryl)(C₀-C₃ alkyl), R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R¹N)—(C₀-C₃ alkyl)-, R^{z}S(O)₂—O—(C₀-C₃ alkyl)-, R^{z}S(O)₂—(R^{x}N)—(C₀-C₃alkyl)-, R^{z}C(O)—, R^{z}S(O)—, and R^{z} S(O)₂—, each of which may be optionally substituted with one or more Y groups as allowed by valency; R⁸ is selected from hydrogen and C₁-C₆ alkyl; X is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃ alkyl), (5- to 10-membered monocyclic or bicyclic heteroaryl)(C₀-C₃ alkyl), R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z} S(O)₂—, each of which may be optionally substituted with one or more Y groups as allowed by valency; R^{x} and R^{y} are independently selected at each occurrence from hydrogen, C₁-C₆ alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is independently selected at each occurrence from hydrogen, halo, C₁-C₆ alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and X is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol. In some embodiments, R⁵ is as described hereinabove. In some embodiments, two or more of the substituents are interconnected to form a fused aromatic ring, a fused aliphatic ring, or a fused heteroaromatic ring.

In some embodiments, R^{A} is

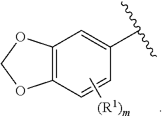

In some embodiments, R^{A} is

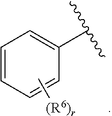

In some embodiments, r is 0. In some embodiments, r is 1. In some embodiments, r is 2. In some embodiments, r is 3. In some embodiments, r is 4. In some embodiments, r is 5. In some embodiments, R⁶ is C₁-C₃ alkyl, for example methyl. In some embodiments, R⁶ is halo, for example fluoro, chloro, or bromo. In some embodiments, R⁶ is nitro. In some embodiments, R⁶ is amino. In some embodiments, R⁶ is C₁-C₃ alkoxy, for example methoxy.

In some embodiments, R^{A} is

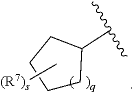

In some embodiments, q is 0. In some embodiments, q is 1. In some embodiments, q is 2. In some embodiments, s is 0. In some embodiments, s is 1. In some embodiments, s is 2. In some embodiments, s is 3. In some embodiments, s is 4.

In some embodiments, Z is —C(O)—. In some embodiments, Z is —N(R⁸)—. In some embodiments, Z is —NH—.

In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8.

In some embodiments, the compound is selected from:

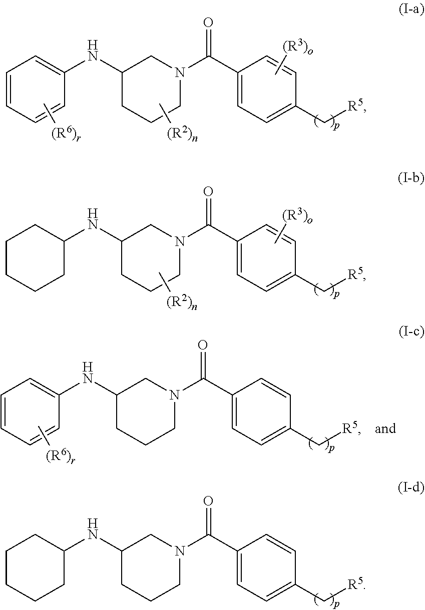

In another aspect, a compound is provided of Formula II:

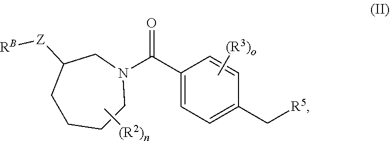

or a pharmaceutically acceptable salt thereof; wherein: R^{B} is selected from

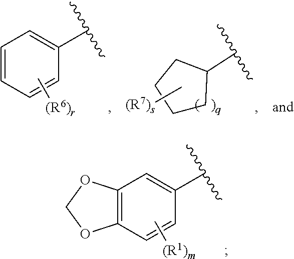

Z is selected from —C(═O)— and —N(R⁸)—; m is 0, 1, 2, or 3; n is 0, 1, 2, 3, or 4; o is 0, 1, 2, 3, or 4; p is 1, 2, 3, 4, 5, 6, 7, or 8; q is 0, 1, or 2; r is 0, 1, 2, 3, 4, or 5; s is 0, 1, 2, 3, or 4; R¹, R², and R³ are independently selected at each occurrence from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z} S(O)₂—, each of which may be optionally substituted with one or more Y groups as allowed by valency; R⁵ is selected from: a) 4- to 6-membered heterocycle having one or two ring heteroatoms independently selected from N, O, or S, wherein the 4- to 6-membered heterocycle is optionally substituted with one or more X groups as allowed by valency; b) 5- to 10-membered monocyclic or bicyclic heteroaryl having one, two, three, or four ring heteroatoms selected from N, O, or S, wherein the 4- to 6-membered heteroaryl is optionally substituted with one or more X groups as allowed by valency; c) —SeR⁹, wherein R⁹ is selected from hydrogen, cyano, alkyl, cyloalkyl, heterocycle, aryl, or heteroaryl, each of which R⁹ may be optionally substituted with one or more X groups as allowed by valency; and d) —NH(C═W)NH₂, wherein W is S or Se; R⁶ and R⁷ are independently selected at each occurrence from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃alkyl), (5- to 10-membered monocyclic or bicyclic heteroaryl)(C₀-C₃ alkyl), R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z} S(O)₂—, each of which may be optionally substituted with one or more Y groups as allowed by valency; R⁸ is selected from hydrogen and C₁-C₆ alkyl; X is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃alkyl), (5- to 10-membered monocyclic or bicyclic heteroaryl)(C₀-C₃ alkyl), R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z} S(O)₂—, each of which may be optionally substituted with one or more Y groups as allowed by valency; R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol. In some embodiments, R⁵ is as described hereinabove.

In some embodiments, R^{B} is

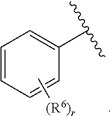

In some embodiments, R⁶ is C₁-C₃ alkyl, for example methyl. In some embodiments, R⁶ is halo, for example fluoro, chloro, or bromo. In some embodiments, R⁶ is nitro. In some embodiments, R⁶ is amino. In some embodiments, R⁶ is C₁-C₃ alkoxy, for example methoxy.

In some embodiments, R^{B} is

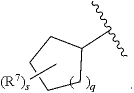

In some embodiments, q is 0. In some embodiments, q is 1. In some embodiments, q is 2. In some embodiments, s is 0. In some embodiments, s is 1. In some embodiments, s is 2. In some embodiments, s is 3. In some embodiments, s is 4.

In some embodiments, R^{B} is

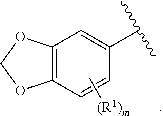

In some embodiments, m is 0. In some embodiments, m is 1. In some embodiments, m is 2. In some embodiments, m is 3.

In some embodiments, Z is as described hereinabove. In some embodiments, p is as described hereinabove.

In some embodiments, the compound is selected from:

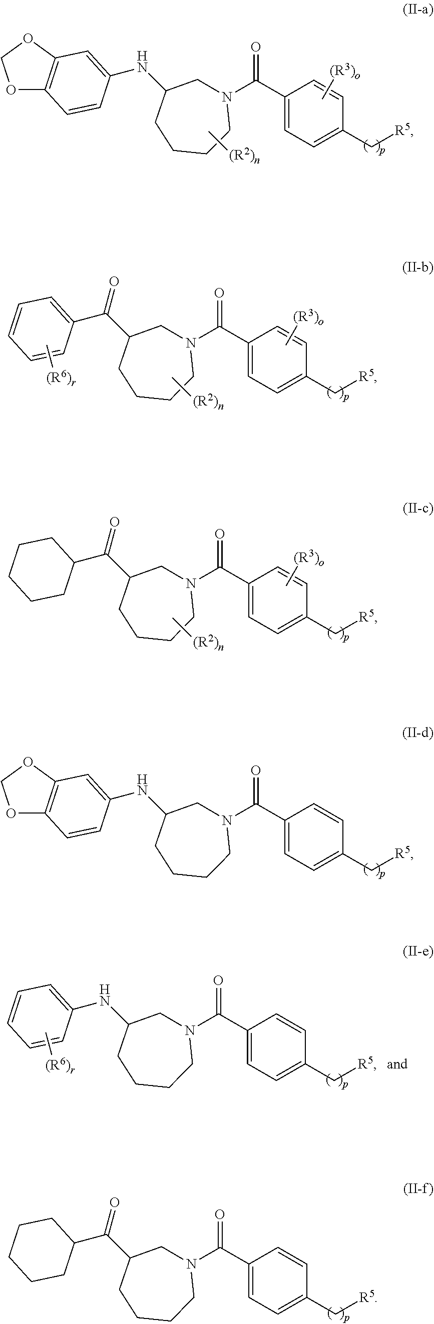

In another aspect, there is provided a compound of Formula III:

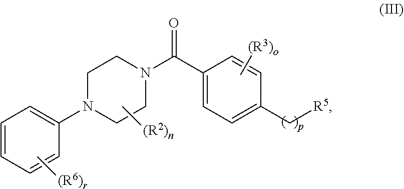

or a pharmaceutically acceptable salt thereof;
wherein: n is 0, 1, 2, 3, or 4; o is 0, 1, 2, 3, or 4; p is 1, 2, 3, 4, 5, 6, 7, or 8; r is 0, 1, 2, 3, 4, or 5; R² and R³ are independently selected at each occurrence from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z} S(O)₂—, each of which may be optionally substituted with one or more Y groups as allowed by valency; R⁵ is selected from: a) 4- to 6-membered heterocycle having one or two ring heteroatoms independently selected from N, O, or S, wherein the 4- to 6-membered heterocycle is optionally substituted with one or more X groups as allowed by valency; b) 5- to 10-membered monocyclic or bicyclic heteroaryl having one, two, three, or four ring heteroatoms selected from N, O, or S, wherein the 4- to 6-membered heteroaryl is optionally substituted with one or more X groups as allowed by valency; c) —SeR⁹, wherein R⁹ is selected from hydrogen, cyano, alkyl, cyloalkyl, heterocycle, aryl, or heteroaryl, each of which R⁹ may be optionally substituted with one or more X groups as allowed by valency; and d) —NH(C═W)NH₂, wherein W is S or Se; R⁶ is independently selected at each occurrence from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃alkyl), (5- to 10-membered monocyclic or bicyclic heteroaryl)(C₀-C₃ alkyl), R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z} S(O)₂—, each of which may be optionally substituted with one or more Y groups as allowed by valency; X is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃alkyl), (5- to 10-membered monocyclic or bicyclic heteroaryl)(C₀-C₃ alkyl), R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z} S(O)₂—, each of which may be optionally substituted with one or more Y groups as allowed by valency; R^{x} and R^{y} are independently selected at each occurrence from hydrogen, C₁-C₆alkyl, C₁-C₆ haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol. In some embodiments, R⁵ is as described hereinabove.

In some embodiments, the compound is of the formula:

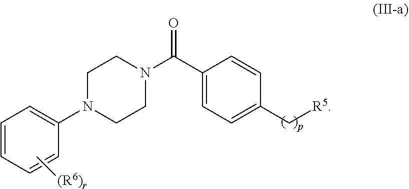

In another aspect, there is provided a compound of Formula IV:

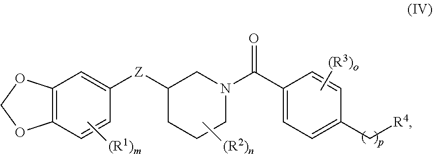

or a pharmaceutically acceptable salt thereof, wherein: Z is selected from —C(═O)— and —N(R⁸)—; m is 0, 1, 2, or 3; n is 0, 1, 2, 3, or 4; o is 0, 1, 2, 3, or 4; p is 1, 2, 3, 4, 5, 6, 7, or 8; R¹, R², and R³ are independently selected at each occurrence from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z} S(O)₂—O—(C₀-C₃ alkyl)-, R^{z} S(O)₂—(R^{x}N)—(C₀-C₃ alkyl)-, R^{z}C(O)—, R^{z}S(O)—, and R^{z} S(O)₂—, each of which may be optionally substituted with one or more Y groups as allowed by valency; R⁴ is selected from: a) 4- to 6-membered heterocycle having one or two ring heteroatoms independently selected from N, O, or S, wherein the 4- to 6-membered heterocycle is optionally substituted with one or more X groups as allowed by valency; b) 5- to 10-membered monocyclic or bicyclic heteroaryl having one, two, or three ring heteroatoms selected from N, O, or S, wherein the 4- to 6-membered heteroaryl is optionally substituted with one or more X groups as allowed by valency; c) —SeR⁹, wherein R⁹ is selected from hydrogen, cyano, alkyl, cyloalkyl, heterocycle, aryl, or heteroaryl, each of which R⁹ may be optionally substituted with one or more X groups as allowed by valency; and d) —NH(C═W)NH₂, wherein W is S or Se; R⁸ is selected from hydrogen and C₁-C₆ alkyl; X is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃alkyl), (5- to 10-membered monocyclic or bicyclic heteroaryl)(C₀-C₃ alkyl), R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z} S(O)₂—O—(C₀-C₃ alkyl)-, R^{z} S(O)₂—(R^{x}N)—(C₀-C₃ alkyl)-, R^{z}C(O)—, R^{z}S(O)—, and R^{z} S(O)₂—, each of which may be optionally substituted with one or more Y groups as allowed by valency; R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some embodiments, R⁴ is 4- to 6-membered heterocycle having one or two ring atoms selected from N, O, and S, and optionally substituted one or more (e.g., one, two, three, or four) X groups.

In some embodiments, R⁴ is azetidinyl, pyrrolidinyl, piperazinyl, or morpholinyl, each of which is optionally substituted with one, two, three, or four X groups.

In some embodiments, R⁴ is selected from:

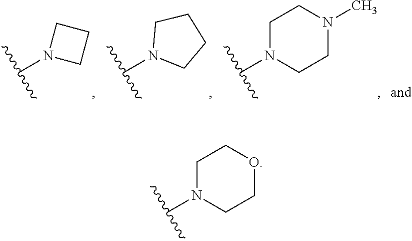

In some embodiments, R⁴ is a 5- to 10-membered monocyclic or bicyclic heteroaryl having one, two, or three ring heteroatoms selected from N, O, and S, and optionally substituted with one or more (e.g., one, two, three, or four) X groups.

In some embodiments, R⁴ is a 5-membered monocyclic heteroaryl having one two, or three heteroatoms selected from N, O, and S, and optionally substituted with one or more (e.g., one, two, three, or four) X groups.

In some embodiments, R⁴ is pyrrolyl, imidazolyl, pyrazolyl, or triazolyl, each of which is optionally substituted with one, two, three, or four X groups.

In some embodiments, R⁴ is selected from:

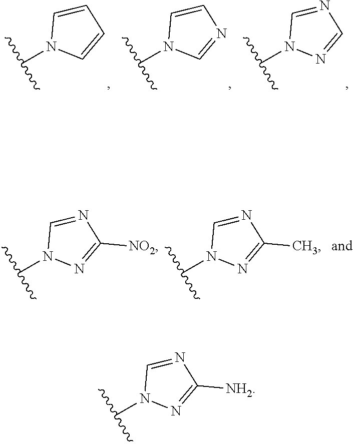

In some embodiments, R⁴ is a 9- to 10-membered bicyclic heteroaryl having one, two, three or four ring heteroatoms selected from N, O, and S, and optionally substituted with one or more (e.g., one, two, three, or four) X groups.

In some embodiments, R⁴ is 1-indolyl optionally substituted with one, two, three, or four X groups.

In some embodiments, R⁴ is selected from:

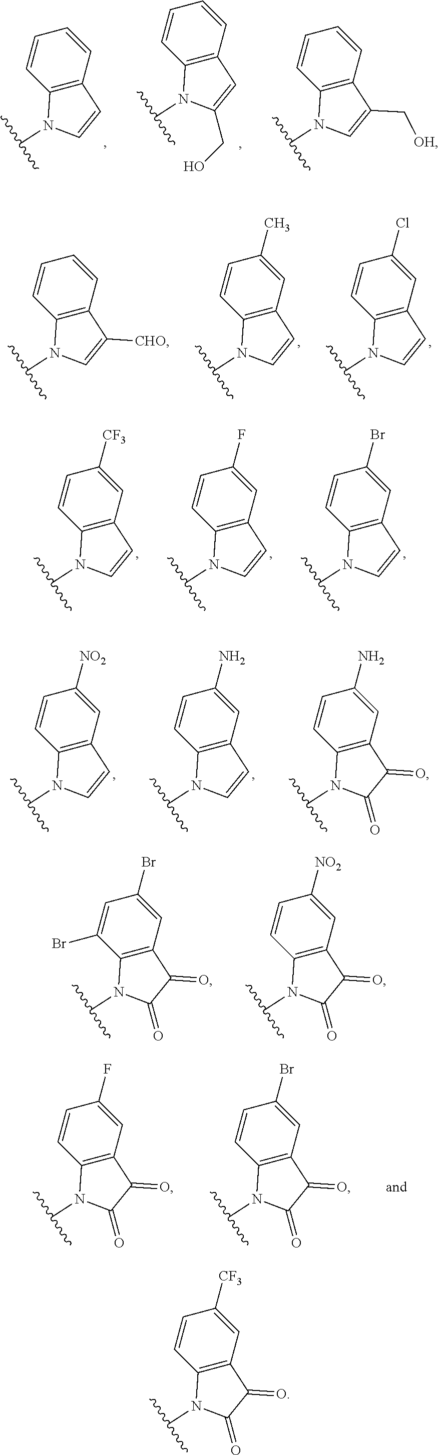

In some embodiments, R⁴ is —SeCN. In some embodiments, R⁴ is —NHC(═S)NH₂.

In some embodiments, the compound is selected from:

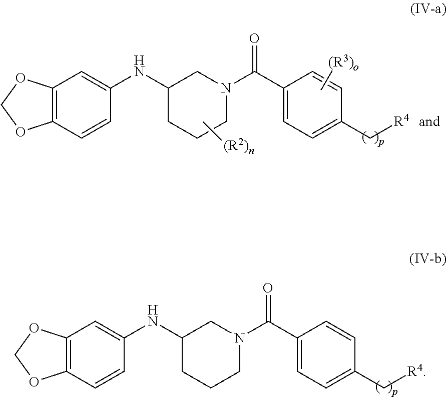

Representative examples of compounds of the present disclosure include, but are not limited to, the compounds provided in Table 1.

TABLE 1
Representative Compounds of the Present Disclosure

| Compound No. | Structure |
|---|---|
| 1 | 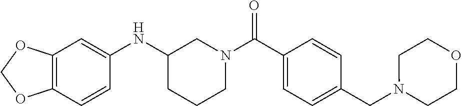 [SMILES: O=C(c1ccc(CN2CCOCC2)cc1)N1CCCC(Nc2ccc3c(c2)OCO3)C1] |
| 2 | 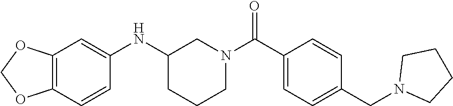 [SMILES: O=C(c1ccc(CN2CCCC2)cc1)N1CCCC(Nc2ccc3c(c2)OCO3)C1] |
| 3 | 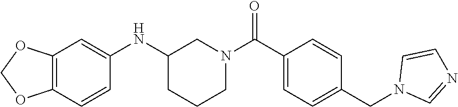 [SMILES: O=C(c1ccc(Cn2ccnc2)cc1)N1CCCC(Nc2ccc3c(c2)OCO3)C1] |
| 4 | 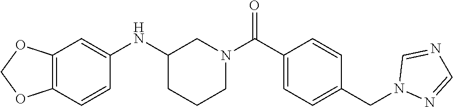 [SMILES: O=C(c1ccc(Cn2cncn2)cc1)N1CCCC(Nc2ccc3c(c2)OCO3)C1] |
| 5 | 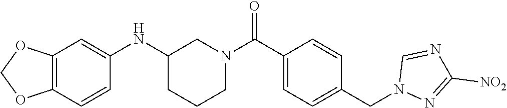 [SMILES: O=C(c1ccc(Cn2cnc([N+](=O)[O-])n2)cc1)N1CCCC(Nc2ccc3c(c2)OCO3)C1] |
| 6 | 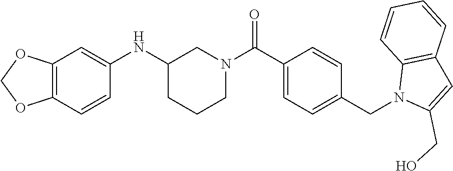 [SMILES: O=C(c1ccc(Cn2c(CO)cc3ccccc32)cc1)N1CCCC(Nc2ccc3c(c2)OCO3)C1] |
| 7 | 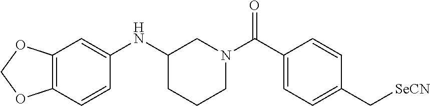 |
| 8 | 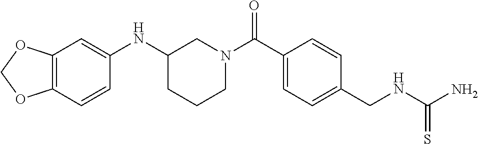 [SMILES: NC(=S)NCc1ccc(C(=O)N2CCCC(Nc3ccc4c(c3)OCO4)C2)cc1] |
| 11 | 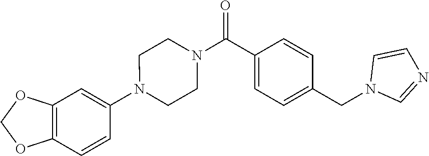 [SMILES: O=C(c1ccc(Cn2ccnc2)cc1)N1CCN(c2ccc3c(c2)OCO3)CC1] |
| 12 | 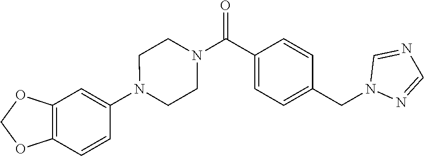 [SMILES: O=C(c1ccc(Cn2cncn2)cc1)N1CCN(c2ccc3c(c2)OCO3)CC1] |
| 13 | 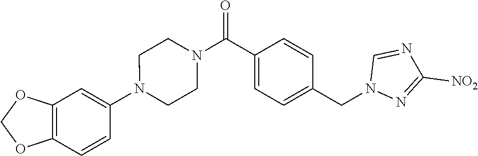 [SMILES: O=C(c1ccc(Cn2cnc([N+](=O)[O-])n2)cc1)N1CCN(c2ccc3c(c2)OCO3)CC1] |
| 14 | 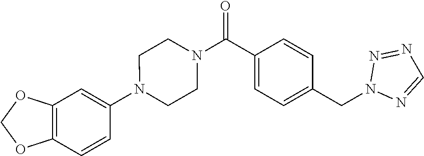 [SMILES: O=C(c1ccc(Cn2ncnn2)cc1)N1CCN(c2ccc3c(c2)OCO3)CC1] |
| 15 | 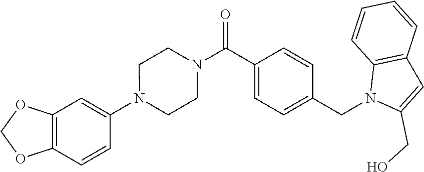 [SMILES: O=C(c1ccc(Cn2c(CO)cc3ccccc32)cc1)N1CCN(c2ccc3c(c2)OCO3)CC1] |
| 16 | 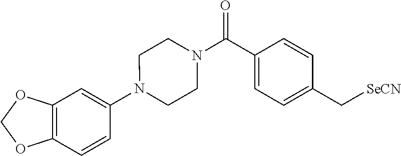 |
| 17 | 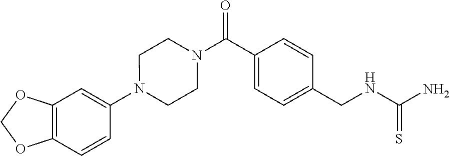 [SMILES: NC(=S)NCc1ccc(C(=O)N2CCN(c3ccc4c(c3)OCO4)CC2)cc1] |
| 18 | 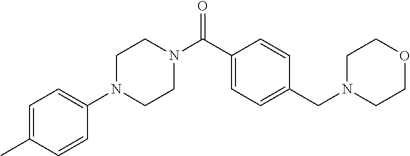 [SMILES: Cc1ccc(N2CCN(C(=O)c3ccc(CN4CCOCC4)cc3)CC2)cc1] |
| 19 | 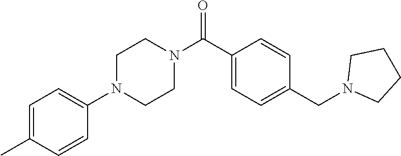 [SMILES: Cc1ccc(N2CCN(C(=O)c3ccc(CN4CCCC4)cc3)CC2)cc1] |
| 20 | 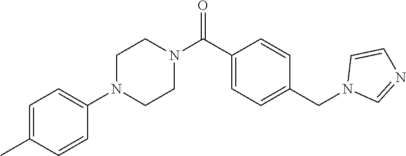 [SMILES: Cc1ccc(N2CCN(C(=O)c3ccc(Cn4ccnc4)cc3)CC2)cc1] |
| 21 | 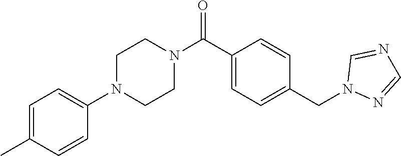 [SMILES: Cc1ccc(N2CCN(C(=O)c3ccc(Cn4cncn4)cc3)CC2)cc1] |
| 22 | 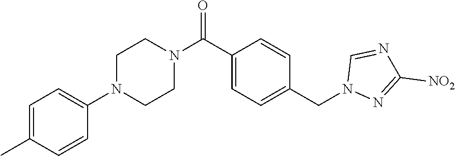 [SMILES: Cc1ccc(N2CCN(C(=O)c3ccc(Cn4cnc([N+](=O)[O-])n4)cc3)CC2)cc1] |
| 23 | 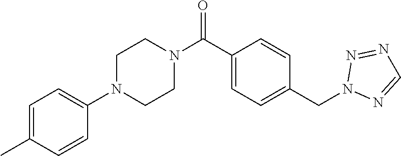 [SMILES: Cc1ccc(N2CCN(C(=O)c3ccc(Cn4ncnn4)cc3)CC2)cc1] |
| 24 | 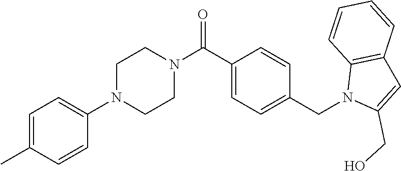 [SMILES: Cc1ccc(N2CCN(C(=O)c3ccc(Cn4c(CO)cc5ccccc54)cc3)CC2)cc1] |
| 25 | 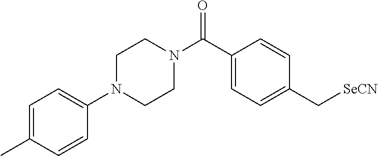 |
| 26 | 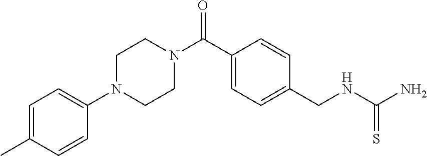 [SMILES: Cc1ccc(N2CCN(C(=O)c3ccc(CNC(N)=S)cc3)CC2)cc1] |

The present disclosure also includes compounds of Formula I, Formula II, Formula III, Formula IV, Formula V, Formula VIa, Formula VIb, Formula VIIa, Formula VIIb, Formula VIIIIa, or Formula VIIIIb, with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tmax, Cmax, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d6-acetone, or d6-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Methods of Treatment

According to some embodiments, the present invention provides a method for preventing or treating a disease or a disorder associated with binding of IGF2BP1 to an RNA in a subject in need thereof, comprising administering to the subject a therapeutically effective amount of a compound or a pharmaceutical composition as described herein, thereby preventing or treating a disease or a disorder associated with binding of IGF2BP1 to an RNA in the subject.

As used herein “insulin-like growth factor-2 mRNA-binding protein 1 (IGF2BP1)” refers to a protein that in humans is encoded by the IGF2BP1 gene. IGF2BP1 is known as a non-catalytic post-transcriptional enhancer of tumor growth upregulated and associated with adverse prognosis in solid cancers.

In some embodiments, the disease comprises a cell proliferation related disease. In some embodiments, the proliferation related disease comprises cancer.

In some embodiments, the cancer comprises any one of a metastatic cancer, a solid tumor, and a liquid tumor.

According to some embodiments, the present invention provides a method for treating or preventing cancer in a subject, comprising administering to the subject a therapeutically effective amount of a compound or a pharmaceutical composition as described herein, thereby treating or preventing cancer in the subject. In some embodiments, the subject is a human subject.

As used herein the term “subject” refers to an individual, or a patient, which is a vertebrate, e.g., a mammal, including especially a human. In some embodiments, the subject is a human. In some embodiments, the subject is a mammal. In some embodiments, the subject suffers from cancer.

In some embodiments, administering comprises an administration route selected from intravenous administration, intraperitoneal administration, subcutaneous administration, or any combination thereof.

In some embodiments, treating comprises: (i) reducing intracellular expression of at least one protooncogene, (ii) preventing or reducing metastasis, or both (i) and (ii).

In some embodiments, the method further comprises a step preceding the administering, comprising determining abundance or levels of any one of: IGF2BP1 transcripts or a protein product thereof, IGF2BP1-RNA complexes, or both, in the subject, wherein an increase in any one of the IGF2BP1 transcripts or a protein product thereof, the IGF2BP1-RNA complexes, or both, in the subject compared to a control, is indicative of the subject being suitable for the treating.

In some embodiments, an increase is by at least 1%, at least 5%, at least 10%, at least 50%, at least 100%, at least 200%, at least 300%, at least 1000%, including any range between) above a pre-determined threshold. Each possibility represents a separate embodiment of the invention.

In some embodiments, the determining is in a sample obtained or derived from the subject. In some embodiments, the sample is a body tissue or a body fluid.

In some embodiments, the method comprises administering the pharmaceutical composition of the invention at least 1 time, at least 2 times, at least 3 times, at least 4 times, at least 5 times, at least 7 times, or at least 10 times per day, or any value and range therebetween. Each possibility represents a separate embodiment of the invention. In some embodiments, the method comprises administering the composition of the invention 1-2 times per day, 1-3 times per day, 1-4 times per day, 1-5 times per day, 1-7 times per day, 2-3 times per day, 2-4 times per day, 2-5 times per day, 3-4 times per day, 3-5 times per day, or 5-7 times per day. Each possibility represents a separate embodiment of the invention.

In some embodiments, the composition of the present invention is administered in a therapeutically safe and effective amount. As used herein, the term “safe and effective amount” refers to the quantity of a component which is sufficient to yield a desired therapeutic response without undue adverse side effects, including but not limited to toxicity, such as calcemic toxicity, irritation, or allergic response, commensurate with a reasonable benefit/risk ratio when used in the presently described manner. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005).

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages may vary depending on the dosage form employed and the route of administration utilized. In one embodiment, the exact formulation, route of administration and dosage can be chosen by the individual physician in view of the patient's condition. [See e.g., Goodman and Gilman's The Pharmacological Basis of Therapeutics, 13th Ed., McGraw-Hill/Education, New York, NY (2017)].

In some embodiments, the effective amount or dose of the active ingredient can be estimated initially from in vitro assays. In one embodiment, a dose can be formulated in animal models and such information can be used to more accurately determine useful doses in humans. In some embodiments, the effective amount or dose of the active ingredient can be estimated by performing a diagnostic method described herein (e.g. a detectable probe based imaging).

The present disclosure also provides methods for treating or preventing cancer in a subject, comprising administering to the subject a therapeutically effective amount of a compound or composition disclosed herein. The methods can further comprise administering one or more additional therapeutic agents, for example anti-cancer agents or anti-inflammatory agents. Additionally, the method can further comprise administering a therapeutically effective amount of ionizing radiation to the subject. Methods of killing a cancer or tumor cell are also provided comprising contacting the cancer or tumor cell with an effective amount of a compound or composition as described herein. In some embodiments, the compounds can inhibit the binding of IGF2BP1 to RNA, for example Kras mRNA. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow pig, or horse, or other animals having an oncological disorder. In some aspects, the subject can receive the therapeutic compositions prior to, during, or after surgical intervention to remove part or all of a tumor.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas. Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocelular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenström macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

### Pharmaceutical Composition

In another aspect of the invention disclosed herein, there is a pharmaceutical composition comprising the compound of the invention, a pharmaceutically acceptable salt thereof or both. In some embodiments, the pharmaceutical composition of the invention comprises a therapeutically effective amount of the compound of the invention and/or any pharmaceutically acceptable salt and/or derivative thereof. In some embodiments, therapeutically effective amount is sufficient for reduction of at least one symptom, or for substantial reduction in the severity and/or inhibition of the progression of a disease, disorder, or condition as described hereinabove. In some embodiments, the therapeutically effective amount can be determined as described hereinabove.

In some embodiments, there is provided herein a composition comprising one or more compounds of the invention, including any salt (e.g. a pharmaceutically acceptable salt), any tautomer, and/or any stereoisomer thereof. In some embodiments, the compound as described hereinabove is the only active ingredient within the composition of the invention (e.g. pharmaceutical composition).

In some embodiments, there is provided herein a pharmaceutical composition comprising a compound represented by Formula V:

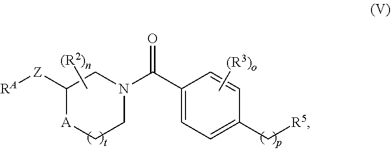

wherein: R^{A} is absent or selected from

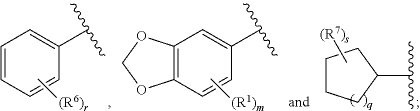

A is selected from CH and N—R, wherein R is

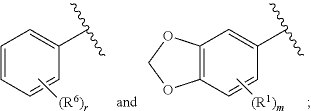

Z is absent, or selected from —C(O)— and —N(R⁸)—, wherein R⁸ is selected from hydrogen and C₁-C₆ alkyl; m is an integer ranging between 0 and 3; each n, o and s is independently an integer ranging between 0 and 4; p is an integer ranging between 0 and 8; q is an integer ranging between 0 and 2; r is an integer ranging between 0 and 5; t is an integer ranging between 1 and 3; R⁵ is selected from: a) 3- to 6-membered aliphatic ring, aromatic ring, or heteroaromatic ring having one or two ring heteroatoms independently selected from N, O, or S, wherein the 3- to 6-membered ring is optionally substituted with one or more X groups as allowed by valency; b) 5- to 10-membered monocyclic or bicyclic aliphatic ring, aromatic ring, or heteroaromatic ring having one, two, three, or four ring heteroatoms selected from N, O, or S, wherein the 5- to 10-membered ring is optionally substituted with one or more X groups as allowed by valency, wherein X is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃ alkyl), (5- to 10-membered monocyclic or bicyclic heteroaryl)(C₀-C₃ alkyl), R^{x}O—(C₀-C₃ alkyl)-, R^{x}S—(C₀-C₃ alkyl)-, (RxRyN)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency, wherein Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol; c) —SeR⁹, wherein R⁹ is selected from hydrogen, cyano, alkyl, cyloalkyl, heterocycle, aryl, or heteroaryl, each of which R⁹ may be optionally substituted with one or more X groups as allowed by valency; and d) —NH(C═W)NH₂, wherein W is S or Se; each X, R¹, R², R³, R⁶ and R⁷ represents one or more substituents, each substituent is independently selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency, or wherein two or more of the substituents are interconnected to form a fused aromatic ring, a fused aliphatic ring, or a fused heteroaromatic ring; each R^{x} and R^{y} is independently selected from hydrogen, C₁-C₆alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is selected from hydrogen, halo, C₁-C₆ alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

In some embodiments, the composition of the invention is a pharmaceutical composition comprising at least one compound of the invention and a pharmaceutically acceptable carrier. In some embodiments, the composition of the invention is a pharmaceutical composition comprising at least one compound of the invention as a first active ingredient and an additional active ingredient.

Non-limiting examples of pharmaceutically acceptable salts include but are not limited to: acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

In some embodiments, the pharmaceutical composition comprises the compound of the invention and a pharmaceutically acceptable carrier. In some embodiments, the pharmaceutical composition comprises a therapeutically effective amount of the compound of the invention and the pharmaceutically acceptable carrier.

In some embodiments, the pharmaceutical composition is in a form of a combination or of a kit of parts. In some embodiments, the pharmaceutical composition of the invention is for use as a medicament.

For example, the term “pharmaceutically acceptable” can mean approved by a regulatory agency of the Federal or a state government or listed in the U.S. Pharmacopeia or other generally recognized pharmacopeia for use in animals, and more particularly in humans. In some embodiments, the compound of the invention is referred to herein as an active ingredient of a pharmaceutical composition.

In some embodiments, the pharmaceutical composition as described herein is a topical composition. In some embodiments, the pharmaceutical composition is an oral composition. In some embodiments, the pharmaceutical composition is an injectable composition. In some embodiments, the pharmaceutical composition is for a systemic use.

As used herein, the term “carrier” refers to a diluent, adjuvant, excipient, or vehicle with which the active ingredient is administered. Such carriers can be sterile liquids, such as water-based and oils, including those of petroleum, animal, vegetable or synthetic origin, such as peanut oil, soybean oil, mineral oil, sesame oil and the like, polyethylene glycols, glycerin, propylene glycol or other synthetic solvents.

Other non-limiting examples of carriers include, but are not limited to: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpineol, terpinen-4-ol, thymol, and thymoquinone. One skilled in the art will appreciate, that a particular carrier used within the pharmaceutical composition of the invention may vary depending on the route of administration.

In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

Water may be used as a carrier such as when the active ingredient has a sufficient aqueous solubility, so as to be administered intravenously. Saline solutions and aqueous dextrose and glycerol solutions can also be employed as liquid carriers, particularly for injectable solutions.

In some embodiments, the carrier is a liquid carrier. In some embodiments, the carrier is an aqueous carrier.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from 0.1% to 99.99999% by weight of the composition/s or the pharmaceutical composition/s presented herein.

In some embodiments, the pharmaceutical composition includes incorporation of any one of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

In some embodiments, the pharmaceutical composition comprising the compound of the invention is in a unit dosage form. In some embodiments, the pharmaceutical composition is prepared by any of the methods well known in the art of pharmacy. In some embodiments, the unit dosage form is in the form of a tablet, capsule, lozenge, wafer, patch, ampoule, vial or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems. In some embodiments, the effective dose is determined as described hereinabove.

In another embodiment, the pharmaceutical composition of the invention is administered in any conventional oral, parenteral or transdermal dosage form.

As used herein, the terms “administering”, “administration”, and like terms refer to any method which, in sound medical practice, delivers a composition containing an active agent to a subject in such a manner as to provide a therapeutic effect. In some embodiments, administering is by an oral administration, a systemic administration or a combination thereof.

In some embodiments, the pharmaceutical composition is administered via oral (i.e., enteral), rectal, vaginal, topical, nasal, ophthalmic, transdermal, subcutaneous, intramuscular, intraperitoneal or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, for oral applications, the pharmaceutical composition or is in the form of a tablets or a capsule, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. In some embodiments, the tablet of the invention is further film coated. In some embodiments, oral application of the pharmaceutical composition or of the kit is in a form of a drinkable liquid. In some embodiments, oral application of the pharmaceutical composition or of the kit is in a form of an edible product.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

In some embodiments, the pharmaceutical composition is for use in the inhibition of disulphide isomerase (PDI) activity. In some embodiments, the pharmaceutical composition is for use in the prevention or treatment of a neurological disorder.

### Pharmaceutically Acceptable Salts

In some embodiments, the compounds of the present invention can exist in free form for treatment, or as a pharmaceutically acceptable salt.

As used herein, the term “pharmaceutically acceptable salt” refers to any non-toxic salt of a compound of the present invention that, upon administration to a subject, e.g., a human, is capable of providing, either directly or indirectly, a compound of this invention or an inhibitory active metabolite or residue thereof. For example, the term “pharmaceutically acceptable” can mean approved by a regulatory agency of the Federal or a state government or listed in the U. S. Pharmacopeia or other generally recognized pharmacopeia for use in animals, and more particularly in humans.

Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified compound in its acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N+(C1-4alkyl)4 salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

In some embodiments, the compounds described herein are chiral compounds (i.e. possess an asymmetric carbon atom). In some embodiments, diastereomers, geometric isomers and individual isomers are encompassed within the scope of the present invention. In some embodiments, a chiral compound described herein is in form of a racemic mixture. In some embodiments, a chiral compound is in form of a single enantiomer, with an asymmetric carbon atom having the R configuration. In some embodiments, a chiral compound is in form of a single enantiomer, with an asymmetric carbon atom having the S configuration as described hereinabove.

In some embodiments, a chiral compound is in form of a single enantiomer with enantiomeric purity of more than 70%. In some embodiments, a chiral compound is in form of a single enantiomer with enantiomeric purity of more than 80%. In some embodiments, a chiral compound is in form of a single enantiomer with enantiomeric purity of more than 90%. In some embodiments, a chiral compound is in form of a single enantiomer with enantiomeric purity of more than 95%.

In some embodiments, the compound of the invention comprising an unsaturated bond is in a form of a trans-, or cis-isomer. In some embodiments, the composition of the invention comprises a mixture of cis- and trans-isomers, as described hereinabove.

In some embodiments, the compounds described herein can exist in unsolvated form as well as in solvated form, including hydrated form. In general, the solvated form is equivalent to the unsolvated form and is encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

In another embodiment, the compositions of the invention take the form of solutions, suspensions, emulsions, tablets, pills, capsules, powders, gels, creams, ointments, foams, pastes, sustained-release formulations and the like. In another embodiment, the compositions of the invention can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in: Remington's Pharmaceutical Sciences” by E. W. Martin, the contents of which are hereby incorporated by reference herein. Such compositions will contain a therapeutically effective amount of the polypeptide of the invention, preferably in a substantially purified form, together with a suitable amount of carrier so as to provide the form for proper administration to the subject.

### Methods of Administration

The compounds described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art. The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications. Dosage can vary, and can be administered in one or more dose administrations daily, for one or several days.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

As used herein, the term “alkyl” describes an aliphatic hydrocarbon including straight chain and branched chain groups. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)₂R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R′″ end group or a —R′NC(N)NR″— linking group, as these phrases are defined hereinabove, where R′, R″ and R′″ are as defined herein. As used herein, the term “azide” refers to a —N₃ group. The term “sulfonamide” refers to a —S(O)2NR′R″ group, with R′ and R″ as defined herein.

The term “phosphonyl” or “phosphonate” describes an —OP(O)—(OR′)₂ group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C₃₋₈ heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C₁₋₆-alkyl, C₁₋₆-haloalkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C₁₋₆-alkyl, C₁₋₆-alkyl, hydroxy-C₁₋₆-alkyl and amino-C₁₋₆-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfone, ethane disulfonic, oxalic, isethionic, HOOC—(CH₂)1-4-COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Material and Methods

### RNA Probe Synthesis

Kras fragments for the 3′UTR RNA probes were synthesized by PCR amplifications of a pGEM-T_Easy plasmid containing part of the Kras cDNA, using the following primer sets (Table 2):

TABLE 2

| | | | | Position/region |
|---|---|---|---|---|
| | F primer | R primer | Length | (Ref sequence- |
| | (SEQ ID NO:) | (SEQ ID NO:) | (bp) | NM_004985) |
| Kras 1 | GCTTTAATACGA | ATTGCACTGTACTCC | 200 | 191/CDS |
| | CTCACTATAGGG | TCATGA (2) | | |
| | ATGACTGAATAT | | | |
| | AAACTTGT (1) | | | |
| Kras2 | GCTTTAATACGA | TTGTGTCTACTGTTC | 199 | 372/CDS |
| | CTCACTATAGGG | TAGAAG (4) | | |
| | TGAGGAGTACAG | | | |
| | TGCAATGA (3) | | | |
| Kras3 | GCTTTAATACGA | TTACATAATTACACA | 200 | 557/CDS |
| | CTCACTATAGGG | CTTTGT (6) | | |
| | GAACAGTAGACA | | | |
| | CAAAACAG (5) | | | |
| Kras4 | GCTTTAATACGA | AAACTCTGGGAATA | 200 | 758/3′UTR |
| | CTCACTATAGGG | CTGGCAC (8) | | |
| | ATACAATTTGTA | | | |
| | CTTTTTTC (7) | | | |
| Kras5 | GCTTTAATACGA | TTAATTTGTTTCACA | 200 | 892/3′UTR |
| | CTCACTATAGGG | CCAAC (10) | | |
| | AAATGCTTATTTT | | | |
| | AAAATGA (9) | | | |
| Kras6 | GCTTTAATACGA | GCTTTAATACGACTC | 200 | 1027/3′UTR |
| | CTCACTATAGGG | ACTATAGGGGTGCAT | | |
| | GTGCATGCAGTT | GCAGTTGATTACTT | | |
| | GATTACTT (11) | (12) | | |

The amplified DNA fragments contain overlapping sequences of approximately 200 bp from the Kras coding region and 5′ end of the 3′UTR with a T7 promoter. These fragments were then used as templates in in vitro transcription reactions, using the MEGA Script T7 High Yield Transcription kit (Invitrogen). For FP experiments, the 5′EndTag Nucleic Acid Labelling System (Vector Laboratories) was used to label the RNA with fluorescein at its 5′ end. Bcl RNA oligo/5′6FAM/CCCGUUGCUUUUCCUCUGGGAAGGAUGGCGCACGCUGGG (SEQ ID NO: 13), was synthesized and HPLC purified by IDT.

### Fluorescence Polarization (FP)

For the small molecule screen, compounds were obtained from the collection maintained at the Grand Israel National Center for Personalized Medicine, consisting of molecules purchased from commercial vendors. Labelled RNA (10 nM) was incubated with recombinant Igf2bp1 protein (400 nM) in FAM-RNA buffer (10 mM Tris pH 8.0, 75 mM KCl, 0.5 mM EDTA, 1 mM DTT, 0.0188 U/μL RNAsin, 0.0005 μg/mL heparin, 0.0001 μg/μL tRNA, 0.01% Triton X-100, 3 mM MgCl₂, 0.05% IGEPAL) at room temperature for 15 min. 7 mL of this reaction mix was aliquoted with GNF WDII (USA) into wells of 1536 well plates, with a final concentration of 20 mM compound or DMSO. The plates were spun down and read in a BMG Pherastar FS (Germany) plate reader plate reader, in FP mode with a 485/520/520 polarization filter, calibrated to 35 mP for free fluorescein with similar total intensity as experimental samples. The G9a FP assay was based on using an S-adenosylhomocysteine (SAH)-FITC probe and antibody to SAH to produce a high FP signal when the antibody binds to SAH-FITC. Unlabeled SAH produced by incubation of G9a enzyme with S-adenosylmethionine (SAM) and peptide substrate competes for the antibody and results in a low FP signal. 100% activity means that G9a is fully active and therefore a low FP signal is observed (SAH is produced).

### FP Data Analysis and Hit Scoring

To determine the quality of the inventors screening assay, the Z′ factor for each plate was calculated with a Z′ factor greater than 0.5 indicating a robust assay suitable for high throughput screening. Compounds that altered the overall fluorescence intensity compared to controls were considered auto florescence and excluded from further analysis. Inhibition was evaluated by calculating percent inhibition relative to the control wells, where % inhibition=(1−((mPComp−mPMin)/(mPMax−mPMin)))×100, where the assay minimum (mPMin) is fluorescence Kras RNA alone and the assay maximum (mAMax) is fluorescence Kras with Igf2bp1 protein. Any small molecule that causes a change of more than three standard deviations from the mean is determined appropriate for further investigation.

### Microscale Thermophoresis (MST)

Recombinant full-length Igf2bp1 protein was labelled with RED-tris-NTA dye using a Monolith NT His-Tag Labelling Kit (NanoTemper) while RRM12, KH12 and KH34 di-domain peptides were labelled using the Protein Labeling Kit RED NHS 2^{nd} generation (NanoTemper). Labelled proteins were spun at 15,000 g for 5 min to eliminate aggregates. All samples were 2× labelled protein mix of Igf2bp1 (200 nM) in PBST and were combined (1:1) with serial dilutions of compound or Kras6 RNA in binding buffer (10 mMTris pH=7.5, 100 mM NaCl, 0.1 mM EDTA, 0.06% IPEGAL, 0.02 μg/μL tRNA, 2 mM DTT, 0.1 μg/μL heparin) in final volume of 20 μL, room temperature for 15 min. Samples were then loaded into standard capillaries (NanoTemper) and read in the Monolith NT.115 scanner (NanoTemper) using the red filter with reading conditions of 95% LED power and 40% MST power, 5 sec fluorescence before, 30 sec MST on, 5 sec fluorescence after and 25 sec delay between samples. Results were analyzed using the MO Control software (NanoTemper) for curve fitting to K_{D}.

### Electrophoretic Mobility Shift Assay (EMSA)

Recombinant Igf2bp1 protein was serially diluted (1:1) starting at an initial concentration of 10,000 nM in 0.75 μg heparin, in a total volume of 13 μL. Fluorescently labelled Kras 6 RNA (750 nM) diluted in reaction buffer (10 mM Tris pH 7.5, 100 mM NaCl, 0.1 mM EDTA, 1 mg/mL tRNA, 0.25% IGEPAL (Sigma), 0.5 mM DTT) was heated to 65° C. for 5 minutes, kept on ice for 1 minute and RNasin (200 U/mL) (ThermoScientific, RiboLock RNase inhibitor) was then added. 2 mL RNA was added to each well protein sample, and after a 30 min incubation at room temperature in the dark, loading dye (3 μL) (30% glycerol, 0.01% bromocresol green) was added to each mixture. For compound inhibition experiments, compounds or DMSO were incubated for 4 hours at 37° C. with the protein prior to adding the RNA. Samples were electrophoresed on native TBE polyacrylamide gels (6% acrylamide/bis-acrylamide 37.5:1, 0.5×TBE) that had been pre-run for 30 min. Gels were run at 120V in 0.5×TBE, at 4° C. in the dark. They were then carefully washed and scanned using a Typhoon FLA9500 imager using Alexa 488 laser and LPB1/2 filter. Igf2bp1-RNA complex formation was quantified using the ImageJ software. Prism6 software was used for curve fitting and K_{D} calculations.

### Tissue Culture

H1299 cells were maintained in RPMI medium, and ES2, RKO, LKR-M-Fl, LKR-M-GFP, and HEK293 cells were maintained in DMEM (Biological Industries—Israel). Both media contained 10% FCS (Biological Industries) and 10 μg/mL ciprofloxacin (Bayer).

### Cell Migration Assay

Cells were seeded in 96 IncuCyte® ImageLock plates (20×10\hat{ }3 cells per well) for 24 hours, to a near confluency of 95%, before the addition of increasing concentrations of compound. After a further 24 hours, wells were scratched using the IncuCyte® 96-well WoundMaker Tool, and the cells cultured for an additional 48 hours using the IncuCyte® S3 Live-Cell Analysis System (Essen BioScience). The plate was imaged at increments of 120 minutes for a period of 48 hours, and then analyzed for relative wound healing.

### Cell Proliferation Assay

ES2, H1299 and HEK293 cells were seeded in a 96 well plate (5×10\hat{ }3 cells per well) for 24 hours before adding the compound at increasing concentrations. After an additional 24 hours, the plate was incubated in an IncuCyte® S3 Live-Cell Analysis System (Essen BioScience). The wells were filmed for 72 hours, and then analyzed for proliferation rate Using IncuCyte® S3 Live-Cell Analysis System (Essen BioScience)

### Split-Luciferase Assay

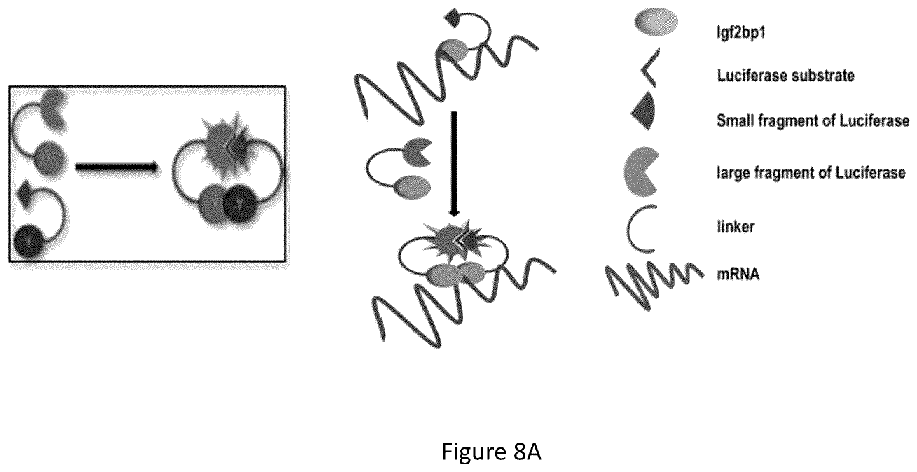

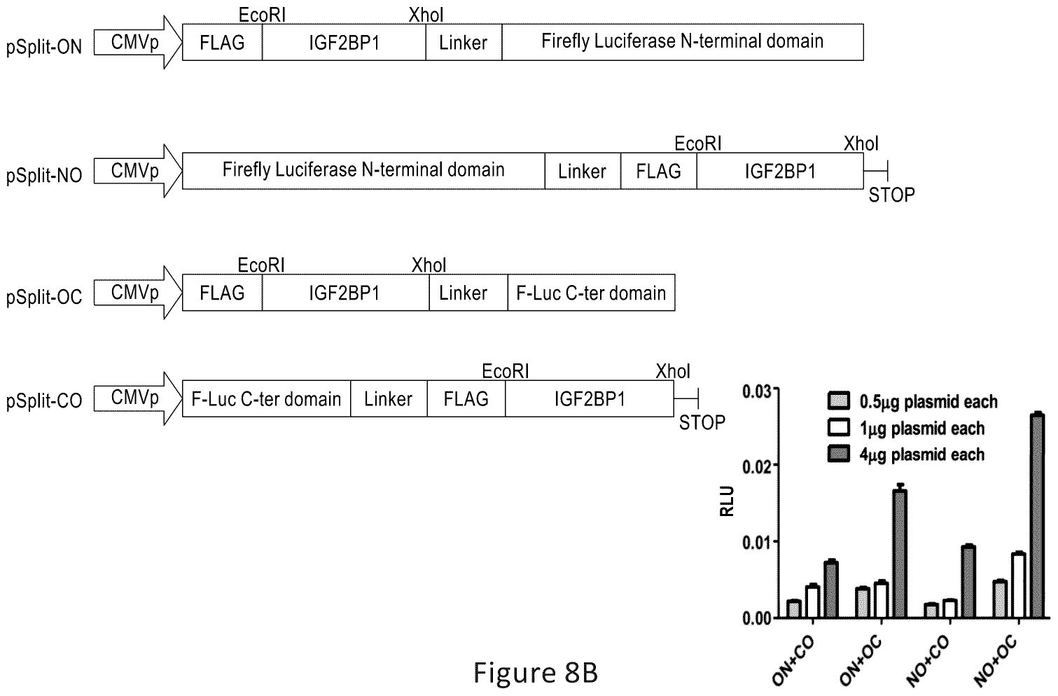

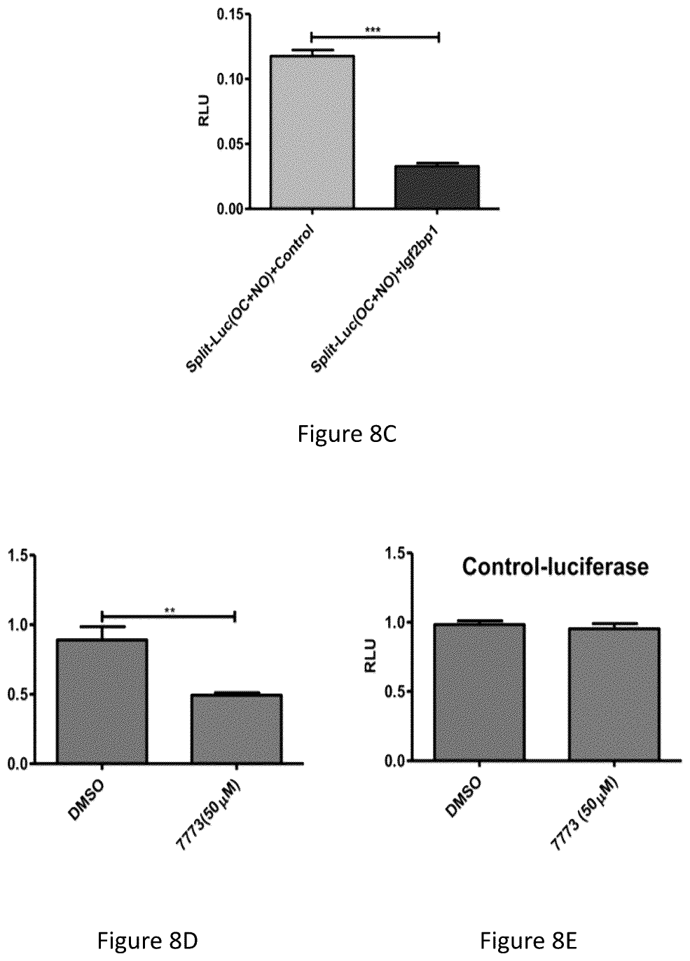

Vectors expressing different fragments of split luciferase reporters with flexible linker were procured from Addgene (catalog #58786, 58787, 58788 and 58789). The vectors were digested with EcoRI and XhoI. EcoRI and XhoI restriction enzyme sites were added to 5′ and 3′ ends of the coding region of IGF2BP1 respectively using PCR. The inventors constructed four split luciferase reporter vectors by cloning IGF2BP1 in different orientation with the luciferase fragments (FIG. 8B). Primers used for the amplification of IGF2BP1 were:

IGF2BP1 Forward Primer-
(SEQ ID NO: 14)
CCGGAATTCGCCACCATGAACAAGCTTTACATCGGCAAC
IGF2BP1 Reverse Primer without STOP Codon-
(SEQ ID NO: 15)
CCGCTCGAGCTTCCTCCGTGCCTGGGCCTGGTT
IGF2BP1 Reverse Primer with STOP Codon-
(SEQ ID NO: 16)
CCGCTCGAGTCACTTCCTCCGTGCCTGGGCCTG

The RKO cells were seeded in DMEM with 10% calf serum 24 hrs prior to the transfection. Transfection was done using Lipofectamine 2000 with reporter plasmids and Renilla-luciferase expressing vector (as internal control). After 24 hrs, cells were lysed using passive lysis buffer (Promega catalog #E1941). The lysates were cleared by centrifugation and its 20 mL volume was mixed with 100 mL of luciferase substrate solution for luminescence recording which was followed by addition of 100 mL of Stop-glo solution (Promega catalog #E1910) with an additional luminescence reading. Both readings were detected by using GloMax 20/20 Luminometer (Promega), and the final value for each sample was the ratio of their two respective readings. To test the effects of 7773, transfected cells with reporter plasmids were washed with PBS after 24 hrs of transfection and incubated with medium containing 7773.

### Western Blot Analysis

Western blots were performed as previously described. For fluorescent westerns, cell lysates were prepared with phosphatase inhibitors (β-Glycerophosphate and Sodium orthovanadate) and a protease inhibitor (cOmplete™, Mini Protease Inhibitor Cocktail, Roche 04693124001), membranes were read using a Li-Core Odyssey laser scanner, and results analyzed using Image Studio Lite software.

The following primary antibodies were used: mouse anti-total ERK (p44/42 MAPK (Erk1/2) L34F12 Cell Signaling 4696s), mouse anti-Kras (CPTC-KRAS4B-2, DSHB) rabbit anti-dpERK, (p44/42 MAPK, Cell Signaling 4370), rabbit anti-alpha/beta tubulin (Cell Signaling 2148), goat anti-rabbit IgG-HRP (Jackson), goat anti-mouse IgG-HRP (Jackson). Secondary antibodies for fluorescent westerns: donkey anti-mouse 800 (Rockland 610-732-124), goat anti-rabbit 680 (Molecular Probes A21076).

qPCR

Primers were calibrated for several Igf2bp1 RNA targets (Table 3):

TABLE 3

| | F primer (SEQ ID NO:) | R primer (SEQ ID NO:) |
|---|---|---|
| Kras | ACCCACCTTGGCCTCATAAAC | ACTGGCATCTGGTAGGCACTC |
| | (17) | (18) |
| c-Myc | GCTGCTTAGACGCTGGATTT | GTCGAGGTCATAGTTCCTGTT |
| | (19) | G (20) |
| CD44 | CTCTGCGGGCTGCTTAGT (21) | TTTATTCGAGGTTGAAAACAG |
| | | TGA (22) |
| SRF | CCTTTCCCATCACCAACTACCT | GCCGCTGCCTGTACTCTTC (24) |
| | (23) | |
| HPRT | GGATTTGGAAAGGGTGTTTATT | TCCCATCTCCTTCATCACATC |
| | C (25) | (26) |

Cells were grown for 12 hours in 12 well plates prior to incubation with the compound at different concentrations for an additional 12 or 24 hours. Total RNA was then extracted using EZ-RNA total RNA isolation kit (Biological Industries) and cDNA prepared from the RNA using the First Strand cDNA Synthesis kit (Quanta bio). Real time PCR was performed with Fast SYBR Green Master Mix (Thermo Fisher Scientific), and cDNA expression analyzed with the Bio-Rad CFX Manager 3.1.

### Growth in Soft Agar

Cells were grown for 12 hours in 6 well plates prior to incubation with the compound or DMSO for an additional 48 hours, 2000 cells were suspended in 0.3% agar-complete medium and then seeded in triplicates onto a 0.6% agar-complete medium base in a 6 well plate. After one week, the colonies were fed with 0.3% agar-complete medium, and after 2 additional weeks, imaged and quantified.

### Cloning

The open reading frame of Igf2bps were cloned into the pET-21(d) plasmid (Novagen) between the Nco1 and Xho1 sites, as previously described. Human Igf2bp1 di-domains RRM12 (M1-P188) and KH34 (P387-A574) cDNA sequences were PCR-amplified from a pCMV6-Entry vector containing the full-length sequence (Origene—NCBI reference sequence NM_006546.4), using the KOD Hot Start Master Mix (Novagen) and primers incorporating NcoI/XhoI restriction sites. After 1-3 h digestion at 37° C. with NcoI/XhoI (NEB) the cDNA sequences were ligated into the bacterial expression vector pETM11 (EMBL) overnight at 16° C. using T4 DNA ligase (NEB) (insert-to-plasmid ratios of 4:1 and 3:1 for RRM12 and KH34 respectively). Plasmids were amplified in DH5α C2987 E. coli (NEB) and successful cloning was confirmed by sequencing (Source Bioscience). Igf2bp1 di-domain KH12 (V194-N369) cDNA sequence was previously cloned in pETM30 (EMBL).

### Protein Expression and Purification.

To induce expression of recombinant Igf2bps-His tag proteins, a single transformed BL21(DE3) colony was grown in LB medium supplemented with ampicillin (100 g/ml) at 37° C. for 7 hours, with agitation. This starter was then transferred to 500 ml fresh LB and incubated with agitation for 1 hour. Protein expression was induced by the addition of IPTG to a final concentration of 0.5 mM for an additional 3 hours. Cells were harvested by centrifugation at 4700 rpm for 20 min at 4° C. The pellet was suspended in HNTA buffer (1M NaCl, 50 mM NaPi buffer pH=7.8 (0.9 M Na2HPO4, 0.1M NaH2PO4), 1% TritonX-100) and Protease Inhibitor Complex (1:100) (APExBIO). Samples were lysed by sonication and spun at 13,000 RPM in cold for 20 min. The collected supernatant was incubated for 2 hours on a high speed rotator at 4° C. with 10 mM imidazole and 0.5 mL Nickel beads (Adar Biotech) which had been prewashed 3× in NTA buffer (0.3 M NaCl, 50 mM NaPi buffer pH=7.8, 1% TritionX-100). The nickel beads were then loaded on a Poly-Prep Chromatography Column (BioRad) and the recombinant protein was eluted through sequential washes of imidazole (1 ml), at the following concentrations: 20 mM, 40 mM, 50 mM, and 250 mM. Samples from all steps of the protein production process were collected and analyzed by SDS-polyacrylamide gel electrophoresis followed by Coomassie blue staining. The fractions which contained the protein were dialyzed at 4° C. overnight using SnakeSkin Dialysis Tubing, 1000 MWCO (Thermo Scientific) in TGKED buffer (50 mM Tris pH=7.5, 50 mM KCL, 0.1 mM EDTA, 0.5 mM DTT). Glycerol (20% v/v) was added to the protein following the dialysis.

The three Igf2bp1 di-domains RRM12, KH12 and KH34 were expressed in BL21(DE3) E. coli cells (Invitrogen). ¹⁵N-labelled N-6×His-RRM12 and —KH34 and N-6×His/GST-KH12 fusion proteins were obtained by growing the cells in M9 minimal media supplemented with ¹⁵NH₄Cl as the sole nitrogen source. Cells were cultured at 37° C. and expression was induced overnight at 18° C. by adding 0.5 mM Isopropyl 3-d-1-thiogalactopyranoside. After cell lysis and sonication in 10 mM TRIS-Base pH 8.0, 10 mM imidazole, 1M NaCl, 5% glycerol, 2 mM 2-Mercaptoethanol, a tablet of cOmplete™ protease inhibitor cocktail (Roche) per 50 mL of buffer, 0.01% Triton-X, 200 μg/mL lysozyme (Sigma), KH12 and KH34 were initially purified by immobilised metal affinity chromatography (IMAC) using a HisTrap™ FF Nickel Sepharose Column (GE Healthcare). RRM12 expressing cells were lysed and sonicated in the same lysis buffer as above added with 8 M urea. Initial purification was performed with a nitrilotriacetic acid agarose matrix (ThermoFisher Scientific) using the same buffers used in IMAC added with 8 M urea. Refolding was obtained by step-wise dialysis at 4° C. After this first step of purification, the tags were removed from the three proteins by overnight cleavage with 5 μM TEV protease at 4° C. in 50 mM TRIS-Base pH 7.5, 150 mM NaCl, 2 mM 2-Mercaptoethanol. The proteins were then loaded onto a cation-exchange Hi-Load SP-Sepharose 26/10 column (GE Healthcare) and eluted by applying a 0-100% gradient of 10 mM TRIS-Base, 1 M NaCl, 2 mM 2-Mercaptoethanol (pH 6.25 for RRM12 and pH 7.3 for KH12 and KH34). The final step of purification was performed using a Hi-Load 16/600 Superdex 75 pg (GE Healthcare), equilibrated with 10 mM Na₂HPO₄ pH 7.4, 50 mM NaCl, 1 mM tris(2-carboxyethyl)phosphine) (TCEP). The purity of the purified protein peak was assessed using SDS-PAGE (Laemmli, 1970), whilst concentration adjusted according to sample absorbance at 280 nm and theoretical extinction coefficient calculated by ProtParam ExPASy (Wilkins et al, 1999). The >95% pure protein samples were stored at −80° C. for use in MSP and NMR assays.

A La protein plasmid was graciously provided by Tilman Helse and purification was performed as previously described.

### Nuclear Magnetic Resonance (NMR) Spectroscopy.

NMR experiments were recorded at 25° C. on a Bruker Avance spectrometer operating at 800 MHz ¹H frequency. ¹H-¹⁵N-Heteronuclear Single Quantum Coherence Nuclear Magnetic Resonance (¹⁵N-HSQC) experiments were performed by adding the 7773 compound (in DMSO) into 50 μM samples of ¹⁵N-RRM12, ¹⁵N—KH12 and ¹⁵N—KH34, obtaining protein-to-7773 molar ratios of 1:1, 2, 4, 8, 14, in 10 mM Na₂HPO₄ pH 7.4, 50 mM NaCl, 1 mM (TCEP), 10% D₂O, 0.02% NaN₃. Some non-specific signal loss is observed during the titration of RRM12 but not of the other di-domains, most likely because of a small amount of aggregation. For ¹⁵N—KH34, an additional equivalent titration was performed in the same buffer but higher salt concentration (150 mM NaCl). All NMR spectra were processed using NMRpipe and analysed with CCPN and TopSpin 4.0.6 (Bruker) software. Chemical shifts perturbations (CSP) were calculated with the formula:

$$CSP = \sqrt{{( {δ}_{{1}_{H}} )}^{2} + {( {δ}_{{15}_{N}} )}^{2}}$$

where δ_{1H} and δ_{15N} are the chemical shift differences of the ¹H and ¹⁵N dimensions respectively. The published ¹⁵N-HSQC resonance assignments were obtained from the Biological Magnetic Resonance Data Bank database and transferred to the ¹⁵N-HSQC spectra.

### Sequence and Protein Alignments.

Primary sequence alignments of KH12 and KH34 were carried out with T-COFFEE multiple sequence alignment server and alignment figures were generated using Jalview using the CLUSTAL X conservation representation. Protein structure alignments were computed in DALI, using the previously reported structures of KH12 and KH34 (PDB access used: 6QEY, 3KRM, 2N8M, 2N8L). All the structure images were obtained with PyMOL Molecular Graphics System 2.0 (Schrödinger, LLC).

### Statistics and Reproducibility

All MST experiments were repeated at least three times, with every point performed in triplicate. K_{D} values are reported with standard deviation. FP experiments for the HTS (greater than 27,000 compounds) were performed once (with an overall Robust Z′ factor of 0.53); 504 molecules showed a standard deviation greater than 3. Of these, 48 compounds passed a further quality control screen (see text) and then were tested in a dose-response assay performed in triplicate in parallel to a counter screen of an unrelated protein/RNA pair using the same FP reaction. Seven molecules demonstrated specific, dose-dependent inhibition of Igf2bp1-Kras 6 RNA binding. One molecule, 7773, was resynthesized twice and inhibited Igf2bp1 binding of Kras 6 RNA with an IC₅₀ of ˜30 μM. EMSA experiments were repeated 3 times, and representative gels are shown. Three biological repeats were used for each point in the wound healing and proliferation assays, and each experiment was performed at least twice. For each RT-PCR experiment, 3 biological repeats were performed, with 3 technical repeats for each sample. Western blots of phosphoERK and Kras proteins were performed twice, with 2 biological repeats for each point. Growth in soft agar was performed twice, with 3 biological repeats for each experimental group.

### Data Availability

In accordance to the UK Medical Research Council policy, the authors will make available data, software and materials related to this study. Plasmids for the expression of IMP1 RRM12, KH12, and KH34 are available from the authors upon request.

### Example 1

### Assay for Kras RNA-Igf2bp1 Binding

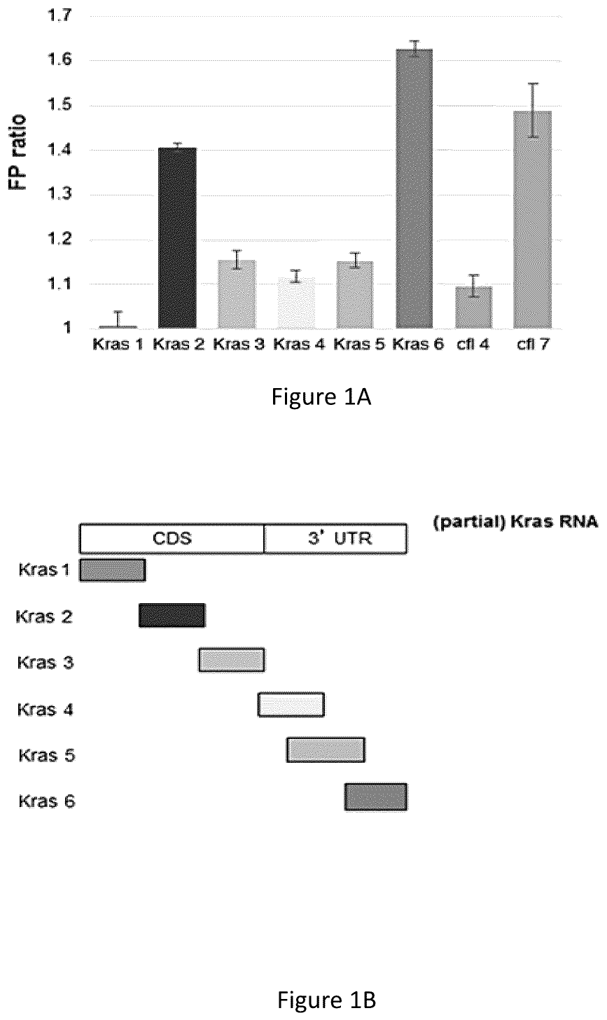

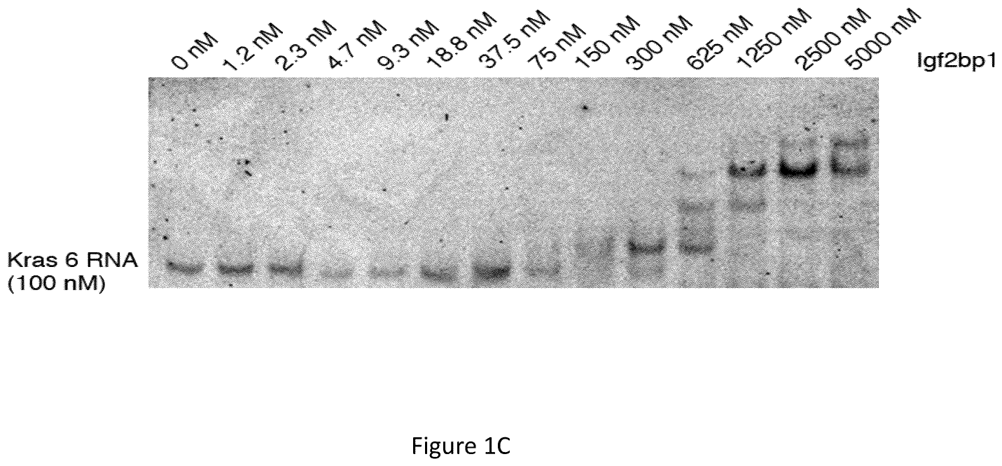

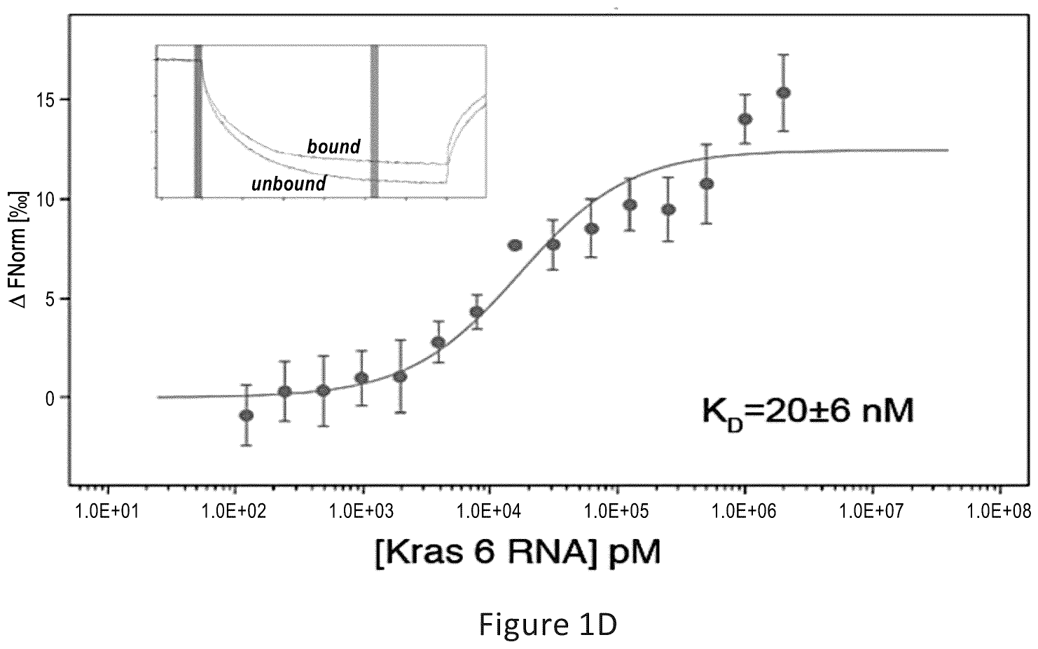

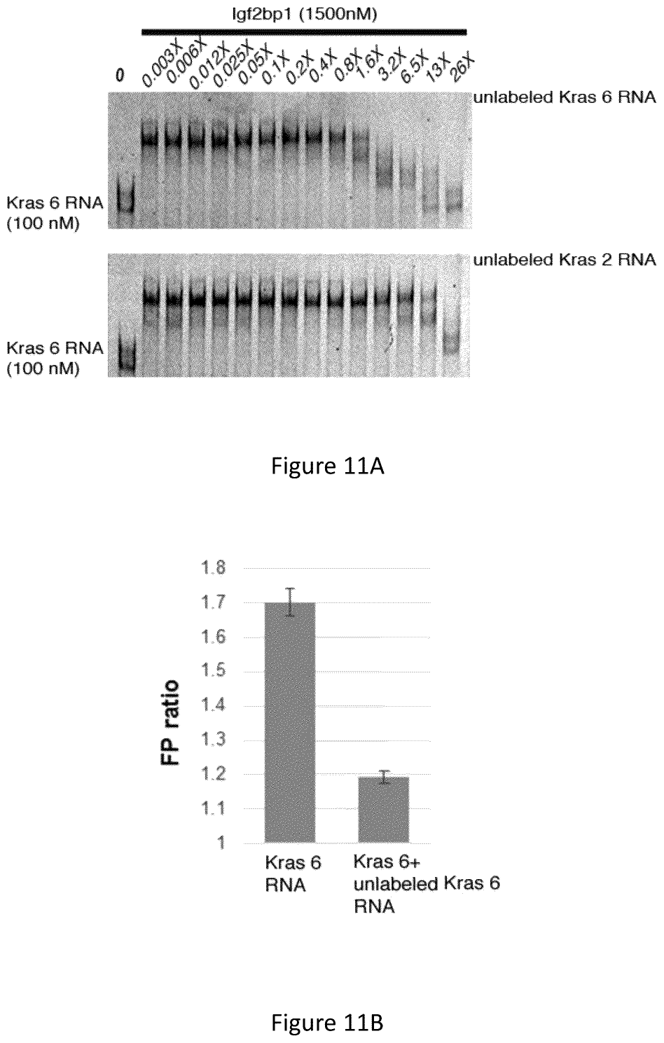

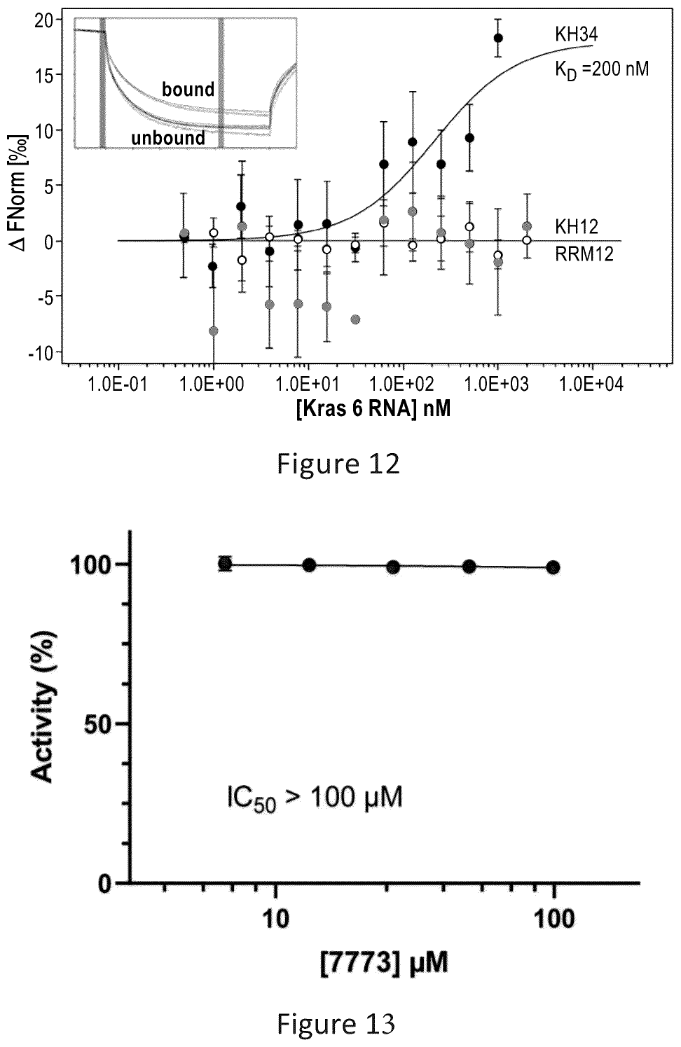

Kras^{G12V} is known to be a driver mutation for lung adenocarcinomas, and the inventors have shown that Igf2bp1 not only binds Kras RNA in mouse lung carcinoma (LKR-M) cells but also synergizes with mutant Kras in promoting human lung adenocarcinomas. Previous work indicated that Igf2bp1 binds the 3′UTR of Kras RNA. The inventors therefore developed an HTS assay to look for small molecules that would inhibit Igf2bp1 binding to Kras RNA, in order to identify molecules that could have therapeutic benefit in preventing Igf2bp1-mediated adenocarcinomas. The inventors synthesized and fluorescently labelled RNA fragments that spanned the coding region and beginning of the 3′UTR of Kras RNA and tested these probes for their ability to bind Igf2bp1 using an FP assay. This homogeneous and rapid technique measures the degree to which a fluorescent molecule is sequestered in solution upon binding to another molecule. As seen in FIGS. 1A-B, one fragment in the 3′UTR, Kras 6, bound Igf2bp1 with high affinity, even when compared with a control RNA fragment from cofilin mRNA (cf7), previously shown to be a functional Igf2bp1 target. Igf2bp1 binding to the Kras 6 fragment was also tested using an electrophoretic mobility shift assay (EMSA, FIG. 1C). As protein concentration increased, an initial high affinity binding event was followed by the appearance of several migration intermediates, suggesting additional recruitment of Igf2bp1 molecules to the RNA-protein complex. This multimerization of Igf2bp1 on RNA had been previously reported for other target RNAs. As expected, inclusion of increasing amounts of unlabeled Kras 6 RNA in the binding reactions inhibited the formation of the protein-RNA complexes seen in the EMSA assay (FIG. 11A), and a similar inhibition of binding was observed in an equivalent FP assay (FIG. 11B). In contrast, competition with unlabeled Kras 2 RNA was less efficient at inhibiting the Igf2bp1-Kras 6 RNA interaction (FIG. 11A). These results confirm that Kras 6 contains a high affinity binding site for Igf2bp1, as observed in the inventors FP experiments, although Kras 2 may represent a secondary binding site, albeit with lower affinity.

Microscale thermophoresis (MST) is a quantitative method for assessing interactions between molecules. Differential migration of fluorescent molecules in an induced thermal gradient can be used to determine the strength of the interaction between Igf2bp1 and an RNA target. The inventors fluorescently labelled the poly-histidine tag of recombinant Igf2bp1-His tag protein and analysed its binding to unlabeled Kras 6 mRNA using MST. In addition to confirming the interaction between Igf2bp1 and Kras 6 mRNA, the inventors found that the dissociation constant (K_{D}) between Igf2bp1 and Kras 6 mRNA is ˜20 nM (FIG. 1D). These results are consistent with the binding strength observed in the inventors EMSA assay (FIG. 1C), even though, in the case of the MST experiment, it is the protein, and not the RNA, that is fluorescently labelled.

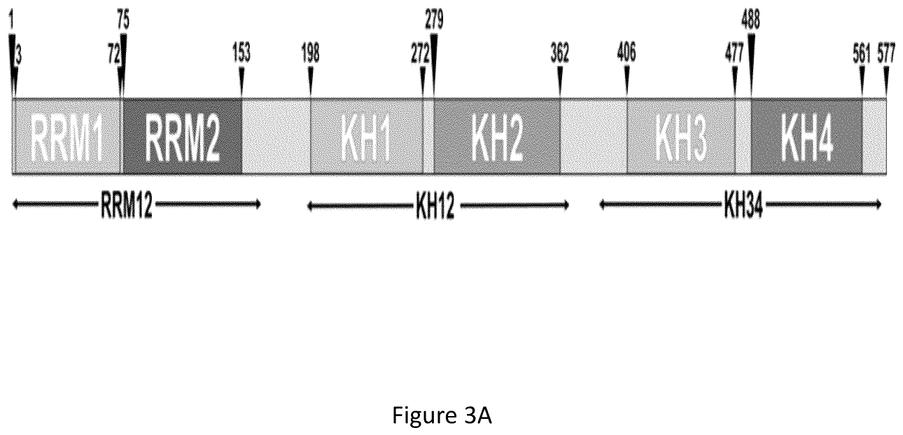

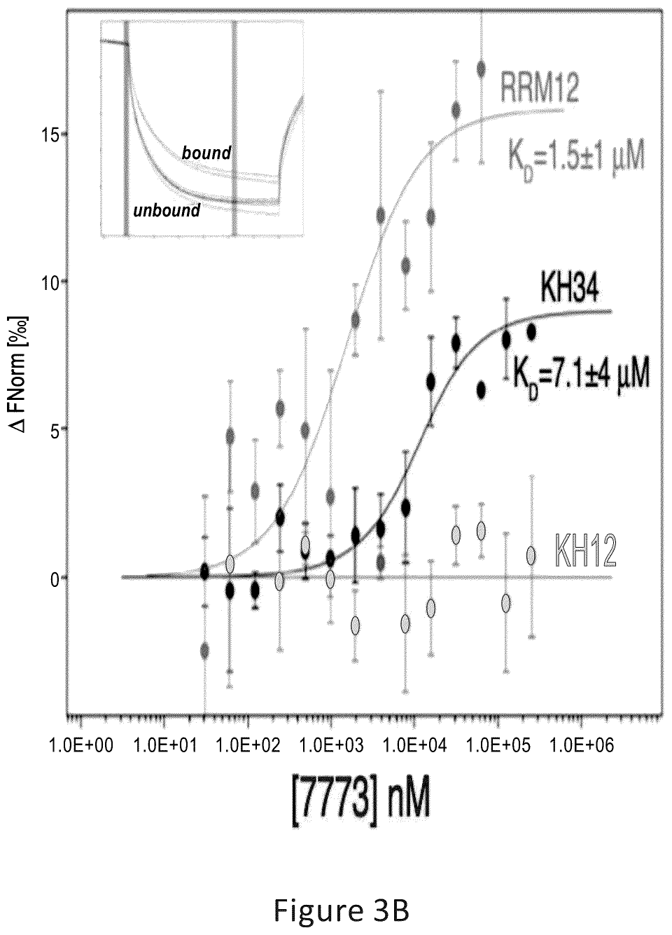

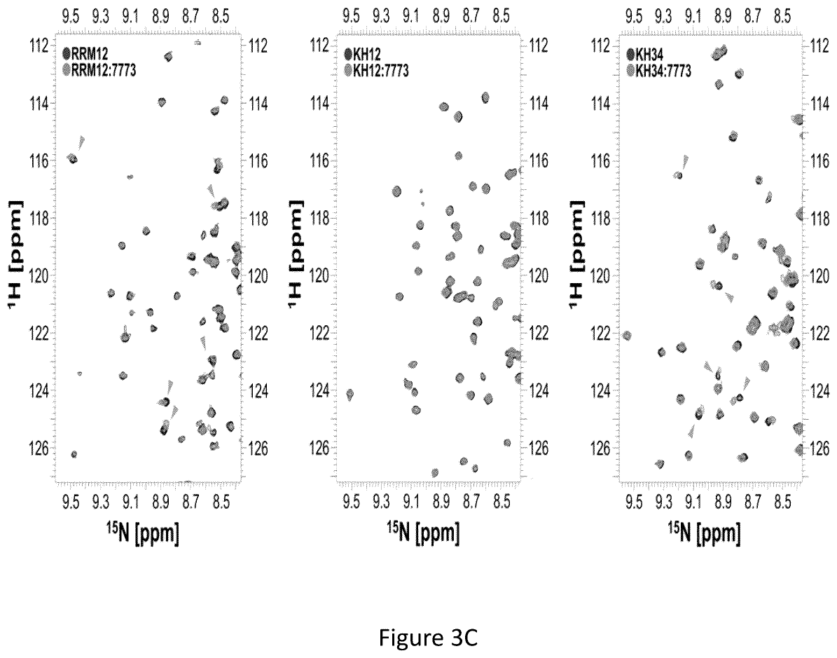

Igf2bps contain 6 putative RNA binding domains, arranged in di-domains from the N- to C-terminus: RRM (RNA Recognition Motif) 1 and 2, KH (hnRNP-K homology domain) 1 and 2, and KH3 and 4 (FIG. 3A). Two of these di-domains, KH12 and KH34, were shown to bind to a range of RNA cognate sequences in vitro and are required for the functional interaction with different targets in cells. It is noteworthy that the functional interaction of Igf2bp1 with some targets, such as β-actin, requires only the KH34 di-domain, while for the interaction with other targets, both KH12 and KH34 are required. To the best of the inventors knowledge, there is no report of the RRM12 di-domain's being essential for the interaction with physiological RNA targets. To characterize which of the RNA binding di-domains of the protein binds Kras 6 RNA, the inventors fluorescently labelled peptides containing the different di-domains and tested these in the MST assay. As shown in Supplemental FIG. 3, Kras 6 RNA bound only to KH34, with a K_{D} of ˜200 nM. This affinity is only ten-fold weaker than that of the entire protein, and, as no binding to the other isolated di-domains is observed, these data suggest that the key interactions between Igf2bp1 and Kras 6 RNA are mediated by KH34. The higher affinity of the full-length protein indicates that additional, weaker interactions with the other domains are also possible. As for many other Igf2bp1 targets in highly proliferating cells, the exact binding site of KH34 is difficult to define, with multiple putative imperfect sites for the individual KH domains present in the Kras6 sequence.

### HTS for Small Molecule Inhibitors of Igf2bp1

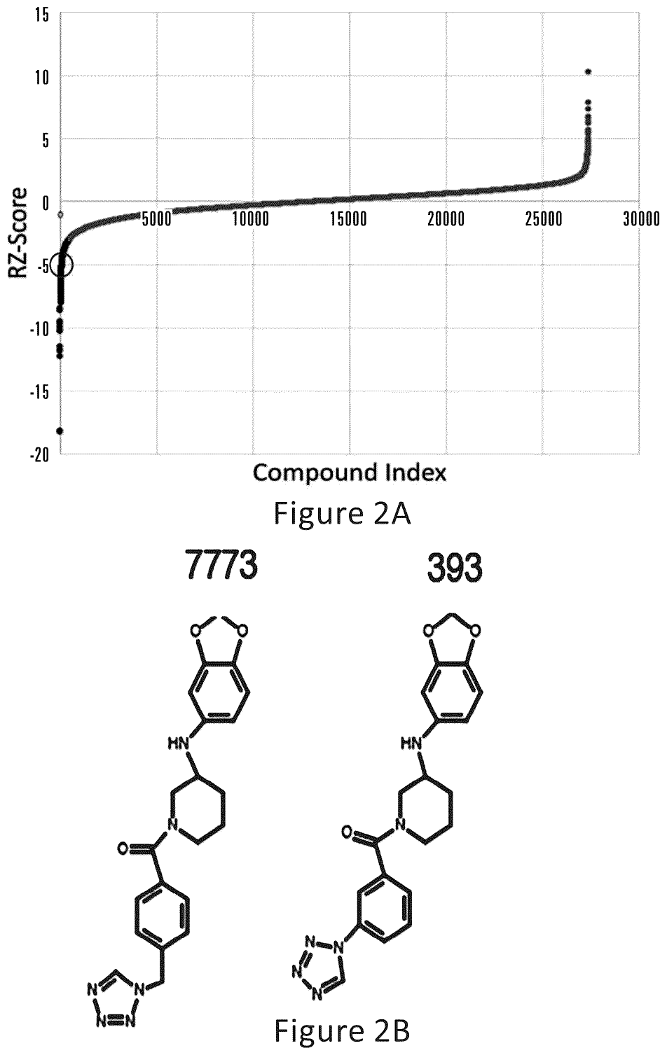

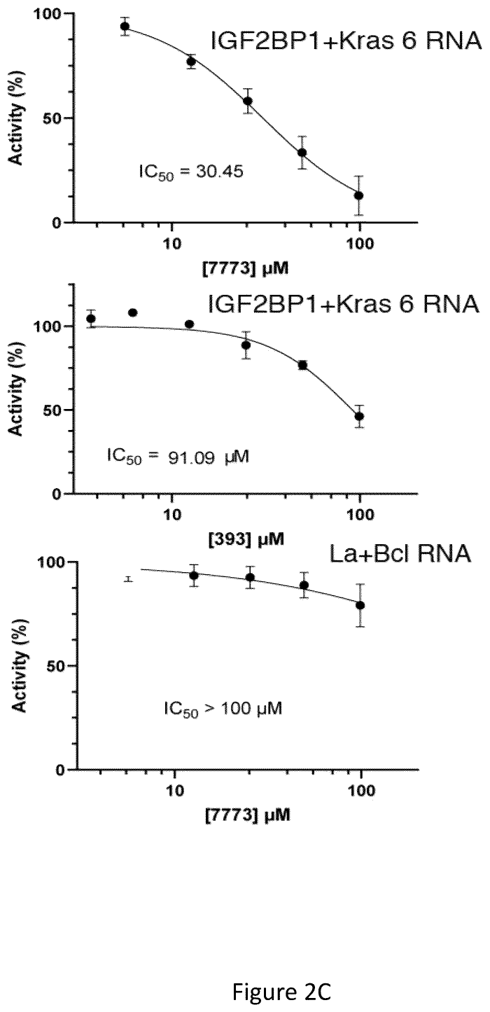

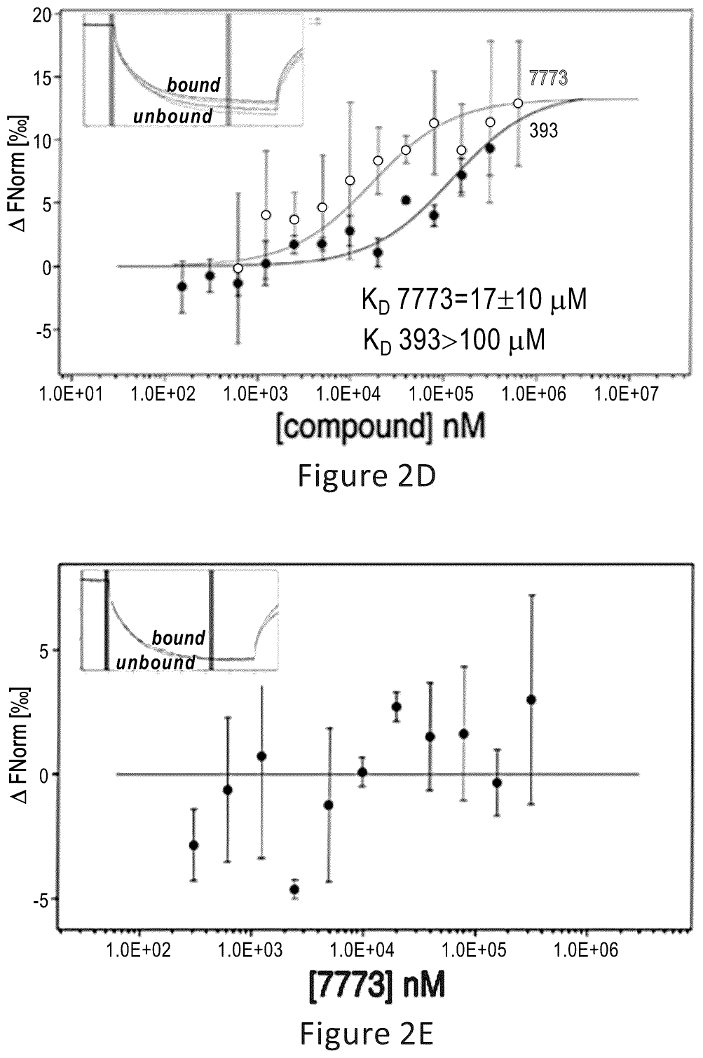

Having identified and characterized a fragment of Kras RNA that binds Igf2bp1, the inventors miniaturized the FP assay to a 1536-well format in order to screen approximately 27,000 compounds (FIG. 2A). The screen was robust, with an overall Robust Z′ factor of 0.53. 504 compounds caused an FP shift of 3 standard deviations or greater. After removing molecules with low or high total fluorescence and correcting for technical plate and assay trending, promiscuity in other assays, and low chemical tractability, 48 compounds were selected for further confirmation by repeat assays and counter screening of an unrelated protein/RNA pair using a similar FP reaction. Seven molecules demonstrated specific, dose-dependent inhibition of Igf2bp1-Kras 6 RNA binding. One molecule, 7773 (FIG. 2B), was resynthesized and inhibited Igf2bp1 binding of Kras 6 RNA with an IC₅₀ of ˜30 μM but had almost no effect on a control RBP (La) binding to its target (Bcl2 RNA) (FIG. 2C) or on a histone methyltransferease protein, G9a, catalyzing methylation of a peptide (FIG. 13). A very similar molecule, 393 (FIG. 2B), showed mild inhibition of Igf2bp1-Kras 6 RNA binding, with an IC₅₀ of ˜90 μM (FIG. 2C). These molecules were used for further studies.

### In Vitro Validation of Igf2bp1 RNA Binding Inhibition

The inventors made use of the MST assay to determine the kinetics of the interaction between the inhibitors, 7773 and 393, and Igf2bp1 protein. As seen in FIG. 2D, increasing concentrations of 7773 cause a significant shift in the curve, yielding a calculated K_{D} of 17 mM. When the less effective 393 compound was used, an approximately seven times lower affinity was observed (K_{D} of ˜120 mM). The K_{D} values calculated from the MST data are consistent with the IC₅₀'s obtained from the dose response FP curves and confirm a direct interaction between the inhibitors and Igf2bp1. Compound 7773 showed no binding in the MST assay to the control RNA binding protein, La (FIG. 2E).

To identify which of the Igf2bp1 structural units may interact with 7773, the inventors tested the ability of the compound to bind the various di-domains using MST. 7773 interacts with both RRM12 and KH34 with K_{D} values of 1.5 and of 7.2 μM respectively (FIG. 3B). These affinities are similar or higher than the one measured for the full-length protein (17 μM), consistent with the compound's binding independently to the individual di-domains. This may result in an underestimate of the K_{D}. It is also worth mentioning that, although the intact protein binds the compound with marginally lower affinity than the individual di-domains, the quality of the data is not the same in the two cases, and this may be a confounding factor. In addition, it is possible that the interaction surface is more exposed in the individual domains. Interestingly, no binding was observed to KH12.

The interactions above were validated using ¹⁵N-HSQC NMR experiments, where the amide proton signals of the individual di-domains were monitored by titration with increasing amounts of 7773. Changes in position and intensity of the peaks are diagnostic of perturbations of the microenvironment of the amide groups and can be used to evaluate binding. While the inventors observed peak shifts of several amide protons in the RRM12 and KH34 titrations, the inventors did not detect similar changes in the KH12 titration (FIG. 3C). In corroboration with the results from the MST experiments, these data indicate that the 7773 compound interacts in vitro with the RRM12 and the KH34, but not the KH12, di-domains.

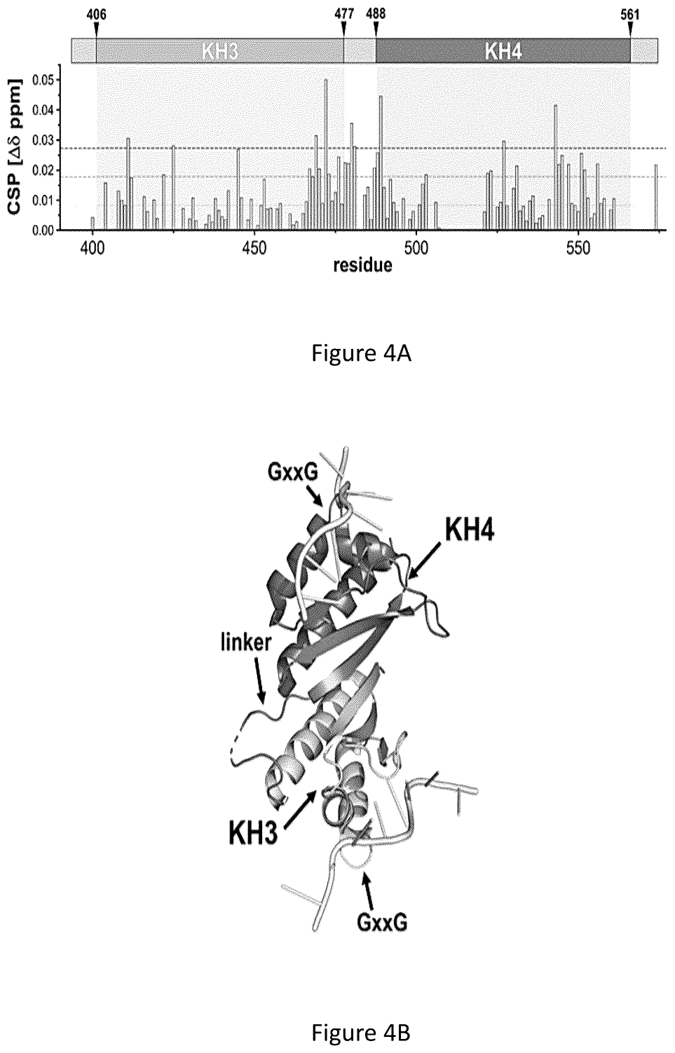

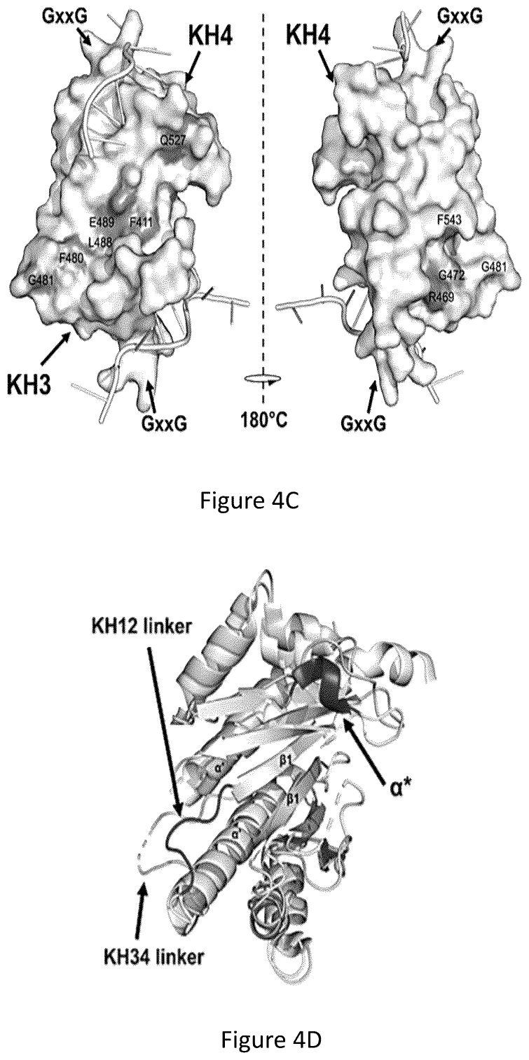

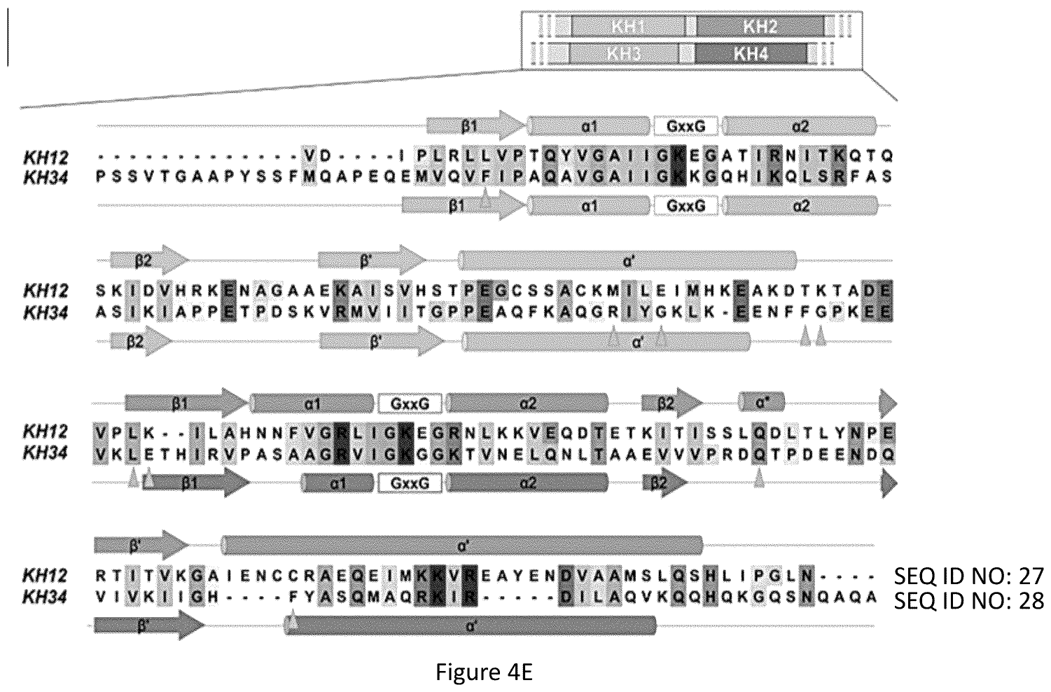

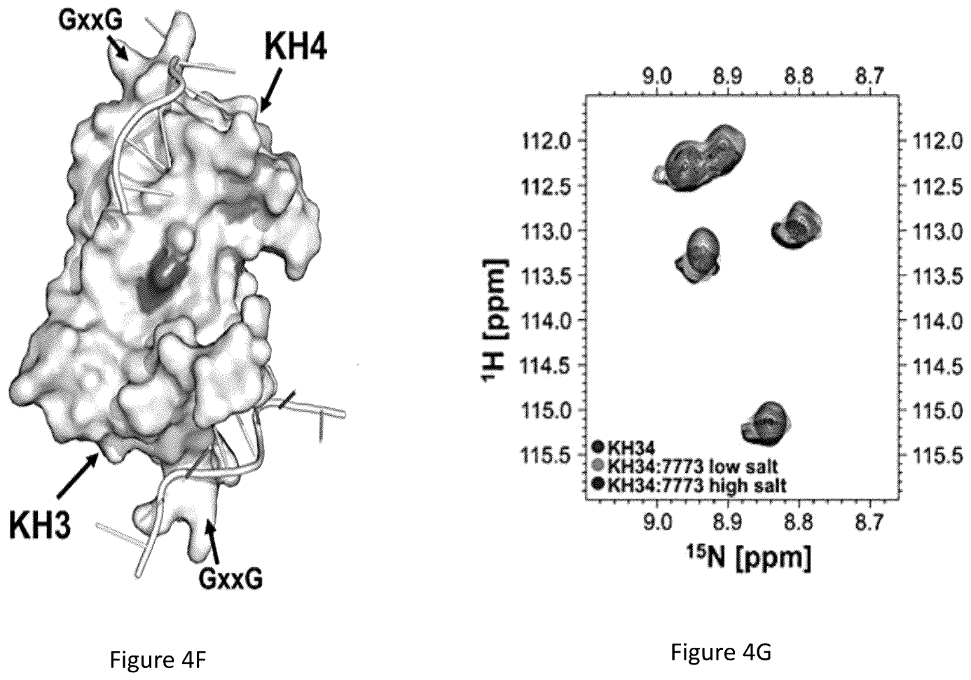

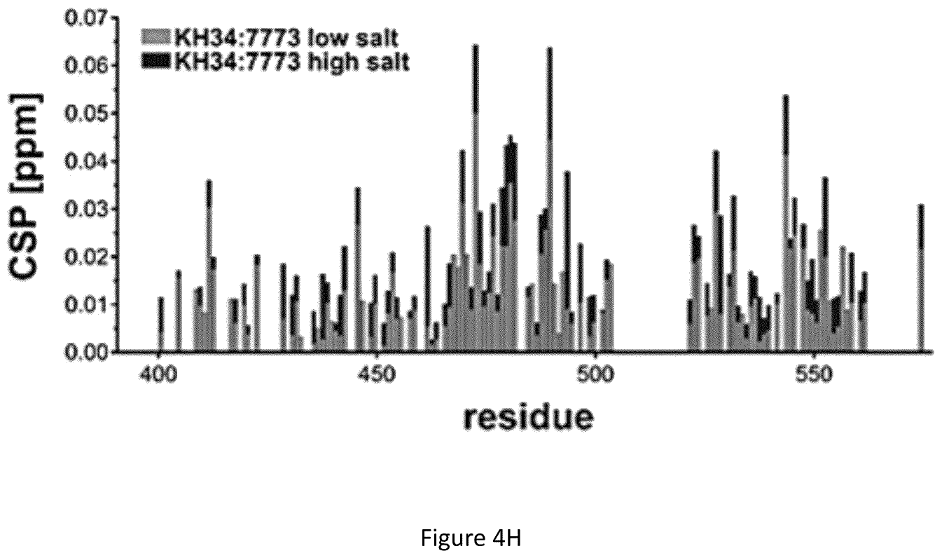

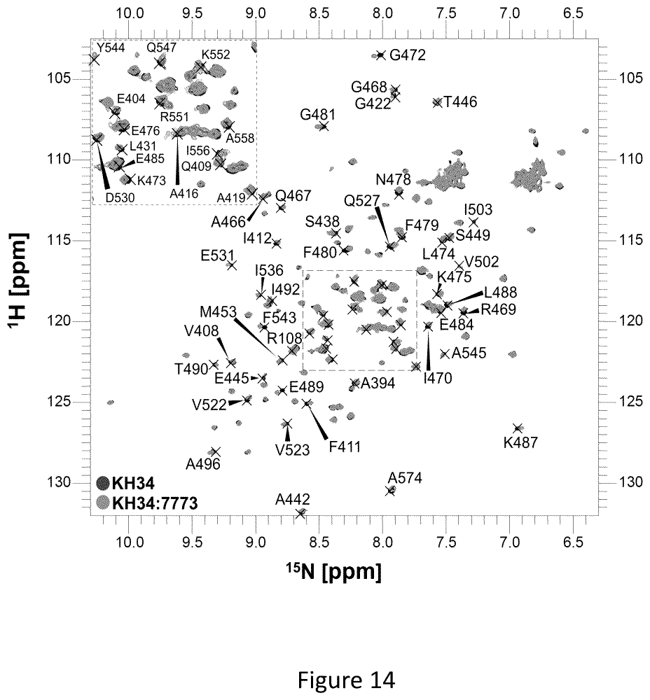

Knowing that Kras 6 RNA binds only to the KH34 domain, the inventors wanted to identify the 7773 binding surface on KH34. The inventors thus transferred the existing assignment of Igf2bp1 KH34 backbone amide onto the inventors ¹⁵N-HSQC spectra (FIG. 14) and identified the amide-protons' resonances that shift upon addition of 7773 (FIG. 3C; FIG. 14). To map the binding site of 7773 onto KH34, the inventors focused on those residues that exhibited a chemical shift perturbation (CSP) above three standard deviations of the average CSP observed (FIG. 4A). The mapping of these onto the structure of KH34 (FIG. 4B) allowed us to visualize the residues whose amide groups' microenvironment is perturbed the most by the inhibitor. The inventors could therefore build a main 7773 binding site onto KH34 (FIG. 4C), an elongated surface at the interface between KH3 and KH4, involving mostly residues of the α′ and β1 of both domains, as well as amino acids in the inter-domain linker. A few more residues were found to significantly shift outside, but close to, the main binding site. The perturbation of these residues might indicate a direct interaction with a chemical moiety of 7773, but could also result from a small conformational rearrangement due to the binding of the molecule onto the KH34 di-domain. Significantly, the inventors data indicate that 7773 binds outside the canonical RNA interacting grooves, the GxxG motifs.

Interestingly, despite the similar inter-domain arrangement and overall structural features of the di-domains, 7773 binds to KH34 but not KH12. In order to understand this difference, the inventors compared the structural features of the two di-domains. Although in both KH12 and KH34, the inter-domain interface is mediated by the interaction of residues of the six-stranded beta-sheets β1 and the alpha helices α′, the conformation of the inter-domain linker, which is a key part of the 7773 interaction surface, is very different. In KH12, the shorter linker is stretched in a linear conformation that spans the distance between KH1 α′ and KH2 β1, while in KH34 it traces a wide turn that allows formation of a small hydrophobic core. Presumably related to the differences in the arrangement of the linker, the angle between the individual KH domains is different in KH12 and KH34, with the KH3 and KH4 domains creating a more open, planar, inter-domain surface. Moreover, KH2 presents a non-conventional one-turn helix α* between β2 and β1 which, although outside of the main binding area, may contribute to occlude the 7773 binding surface (FIG. 4D). In addition to the above structural differences, most of the residues in the interaction surface are different in KH12 and KH34. The mapping of the 7773-dependent chemical shift changes on the sequence alignment of the KH12 and KH34 indicated that seven of the nine significantly perturbed residues are different in the two di-domains (FIG. 4E). The analysis of the structure and amino acid composition of the 7773-KH34 interaction surface together explain the difference in binding between KH12 and KH34.

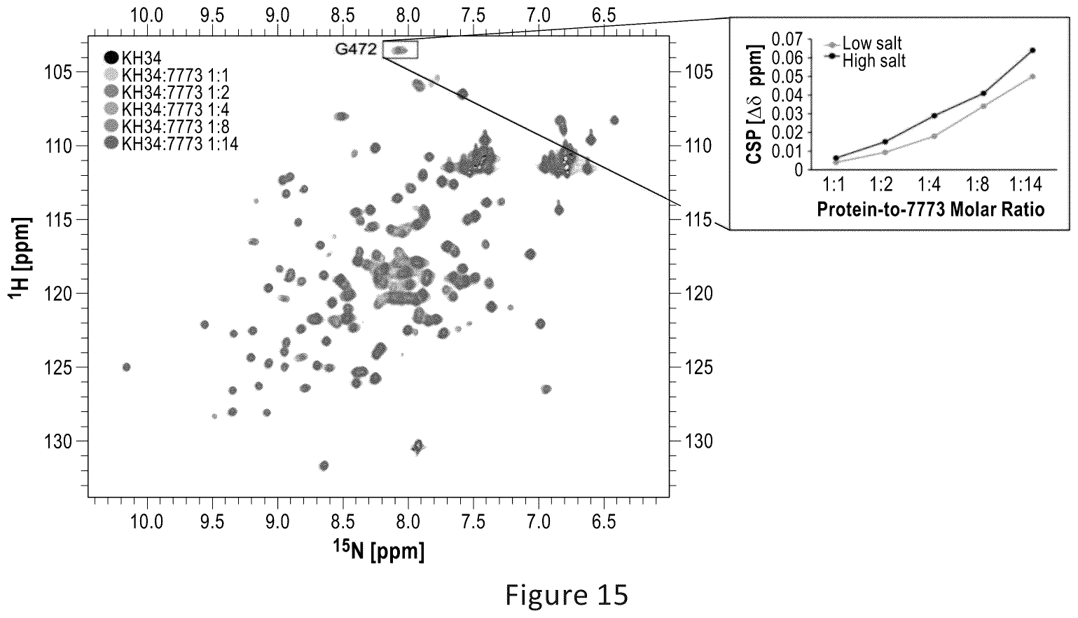

The inventors also evaluated the overall properties of the 7773-KH34 interaction surface. An analysis of residue type (polar, charged and hydrophobic) revealed that the inhibitor binding region is predominantly hydrophobic (FIG. 4F). To validate this observation and assess the contribution of the hydrophobic contacts to binding, the inventors probed the salt-dependency of the interaction in ¹⁵N-HSQC KH34 experiments. The inventors titrated 7773 into a KH34 sample in a buffer with a higher but physiological salt concentration and compared the results with the lower salt titration discussed above (FIG. 4G and FIG. 15). Comparison of the chemical shift changes caused by 7773 binding at 50 mM and 150 mM salt showed that even a relatively modest three-fold increase in salt concentration shifted the protein resonances towards the bound position (25% on average) (FIG. 4H), which is best visualized by plotting the shift of the representative G472 residue during the titration (FIG. 15). This increase in binding affinity confirms the importance of the hydrophobic residues and provides a general insight into the forces driving the interaction.

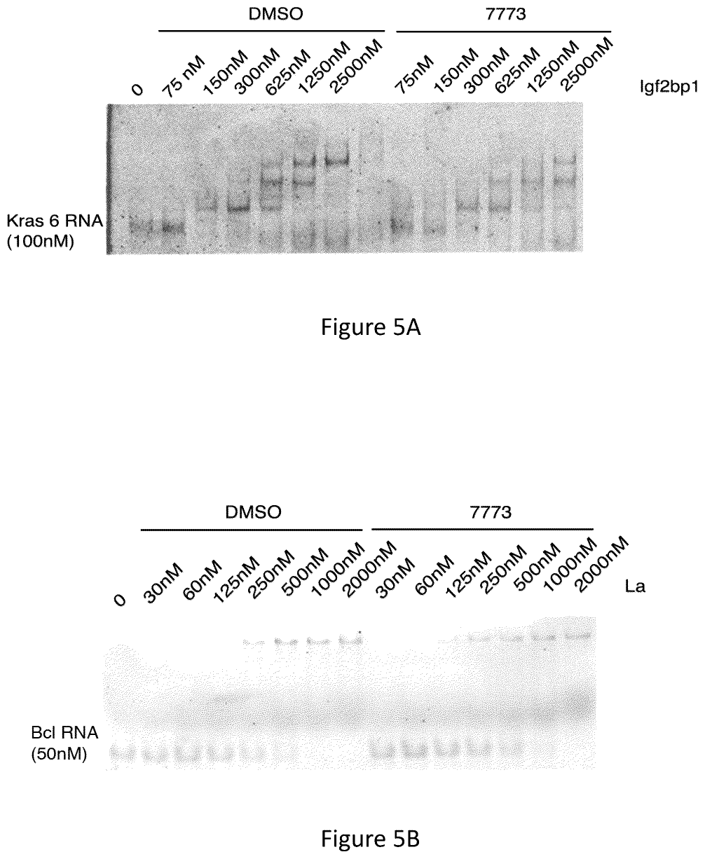

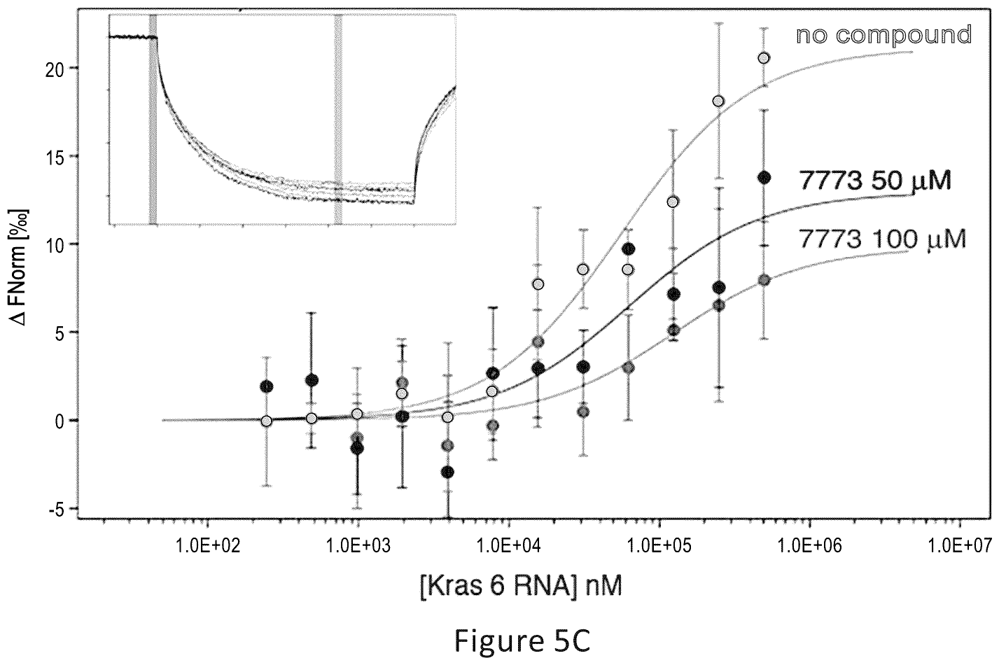

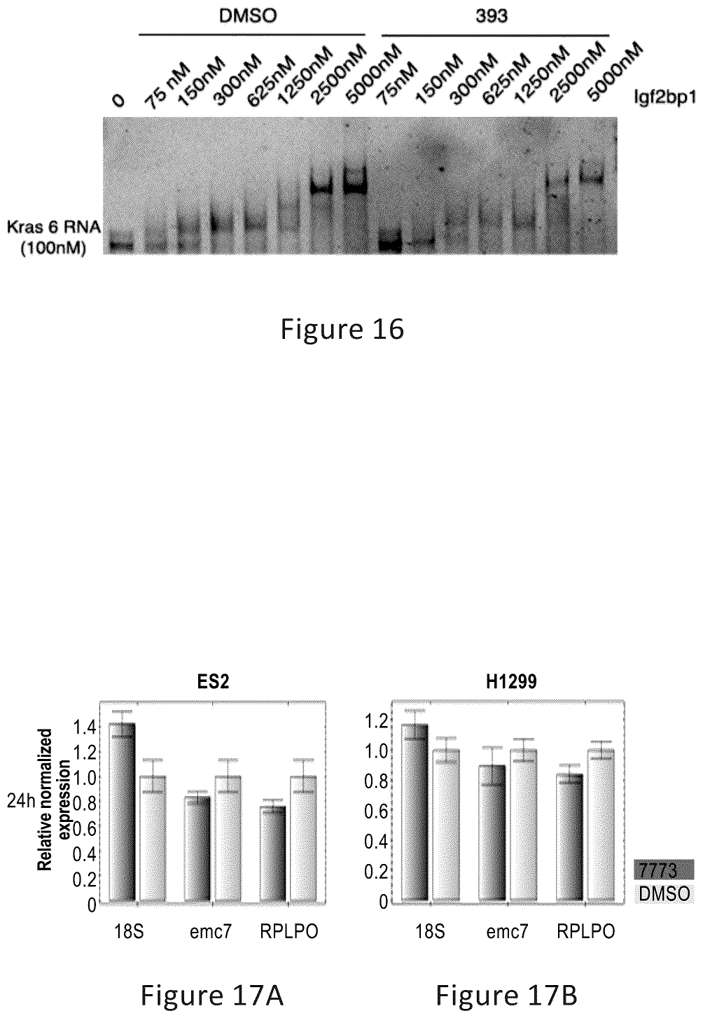

To validate that 7773 disrupts Igf2bp1 binding to its RNA target, the inventors made use of both EMSA and MST assays. Igf2bp1 protein was pre-incubated with either 7773 (dissolved in DMSO) or DMSO and then tested for its ability to bind Kras 6 RNA, as assayed by gel shift. As Igf2bp1 concentrations are increased, slower-migrating RNA-protein complexes appear on the gel in a protein concentration-dependent manner, even in the presence of DMSO (compare FIG. 5A to FIG. 1C). In the presence of the inhibitor 7773, however, a clear reduction in slower-migrating bands is observed at all of the protein concentrations tested. The 393 inhibitor, which showed a much lower affinity for binding to Igf2bp1 in the MST assay, exhibited a lower, but observable, inhibition of RNA binding in the EMSA assay (FIG. 16). Consistent with the results observed with FP, neither 7773 nor DMSO affected the ability of La protein to retard the mobility of its target RNA, Bcl2 (FIG. 5B). It is worth noting that La binds RNA mainly via a La domain and an RRM domain. La protein therefore acts both as a general control and an ‘RRM fold’ control in the assays above.

To further verify the activity of 7773, the inventors tested its ability to inhibit Igf2bp1 binding to Kras 6 RNA in a dose dependent manner using the MST assay. As seen in FIG. 5C, increased concentrations of 7773 led to smaller shifts in the thermophoresis curves, as expected from the reduction in the number of Igf2bp1 molecules, not complexed with 7773, that are available to bind Kras 6 RNA. These results validate those obtained from the FP, NMR, and EMSA assays and suggest that 7773 is a direct, effective, and selective inhibitor of Igf2 bp1 RNA binding in vitro.

### Inhibition of the Other Igf2 bp Paralogues

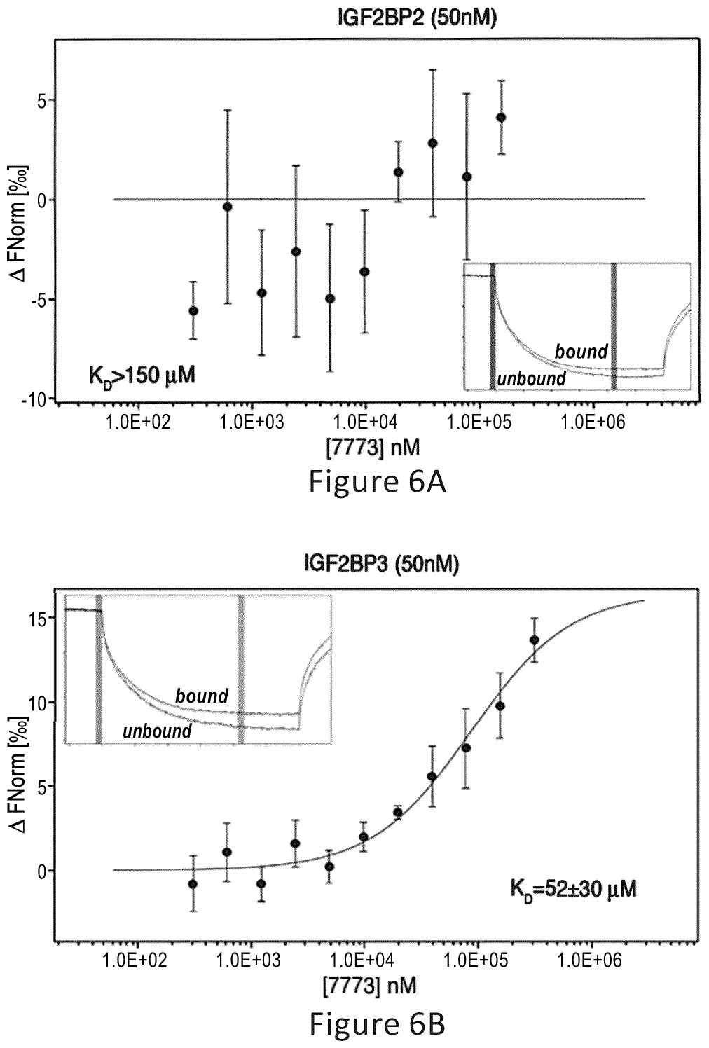

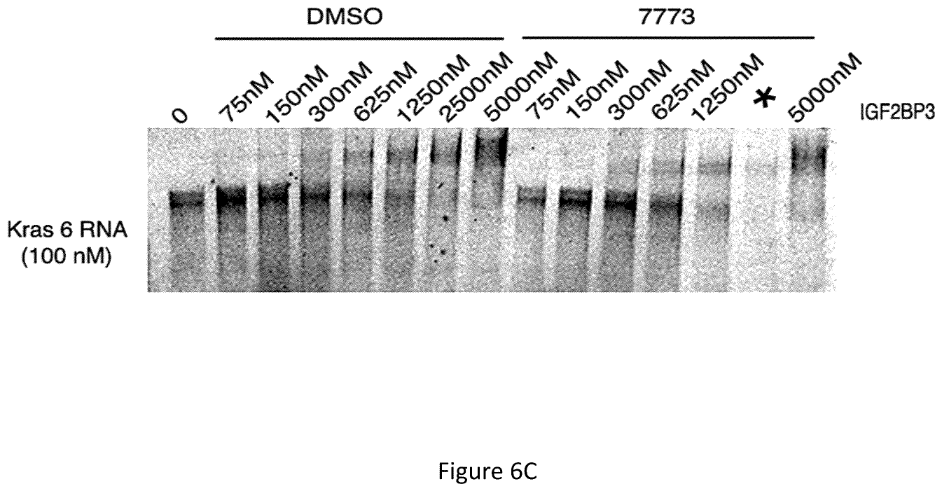

Given the high degree of similarity among the Igf2 bp paralogues, the inventors explored the specificity of 7773 for Igf2 bp paralogs using the MST assay. 7773 did not significantly bind Igf2bp2, even at concentrations that almost saturate the binding to Igf2bp1 (FIG. 6A). 7773 did bind Igf2bp3 (FIG. 6B), although the K_{D} was significantly higher (52 mM) than that observed with Igf2bp1 (17 mM). The gel shift assay also confirmed that 7773 weakly inhibited Igf2bp3 binding of Kras 6 RNA compared to the DMSO control (FIG. 6C). These results indicate that 7773 inhibits in vitro Kras RNA binding of both Igf2bp1 and Igf2bp3, but not Igf2bp2.

### 7773 Targets Igf2bp1 Activity in Cells

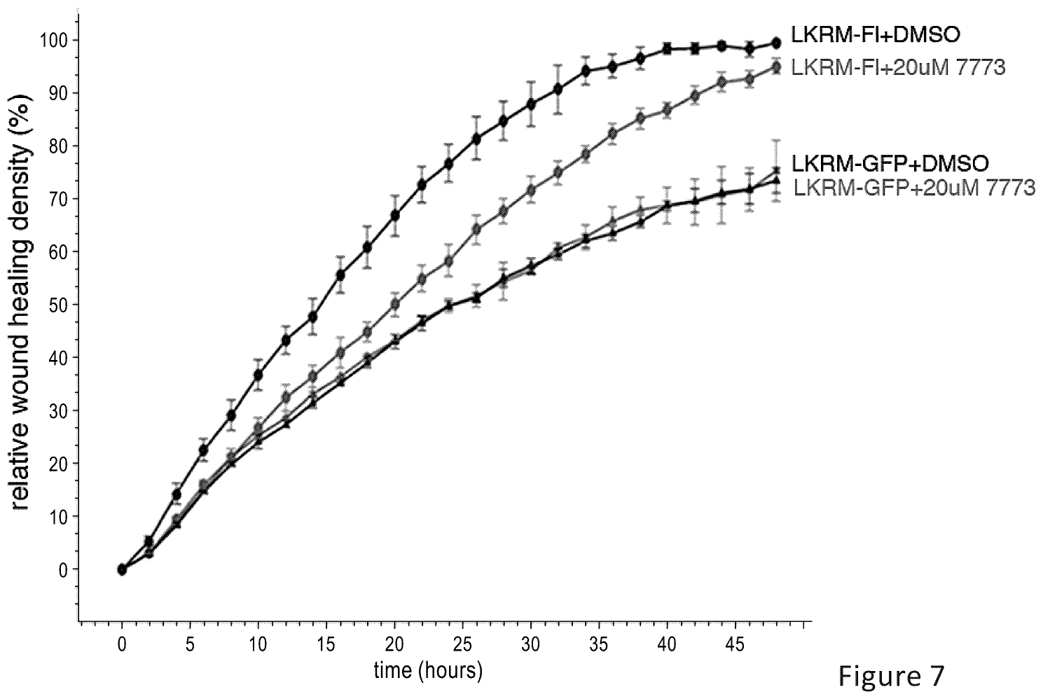

The inventors made use of two different assays to test whether 7773 also targets Igf2bp1 in cultured cells. First, the inventors followed how treating cells with the compound effects their migration in a wound healing assay. Previous work demonstrated the involvement of Igf2bp1 in helping mediate cell migration in lung adenocarcinoma cells, both in cultured cells and in mice. Here, mouse LKR-M cells overexpressing either GFP or human Igf2bp1-GFP were cultured with either DMSO or 7773. As seen previously, exogenous human Igf2bp1-GFP enhanced wound healing when compared to expression of GFP alone (FIG. 7). This enhancement was significantly inhibited by exposure of the Igf2bp1-GFP-expressing cells to 7773. The migration of LKR-M cells expressing GFP alone, however, was unaffected by incubation with 7773 (as compared to DMSO), suggesting that there were no off-target effects of 7773 incubation. Notably, the only Igf2 bp paralogue endogenously expressed in LKR-M cells is Igf2bp2, which is not bound by 7773 in vitro (see FIG. 6). These results indicate that human Igf2bp1 activity, which helps mediate cell migration when expressed in LKR-M cells, is directly and specifically targeted and inhibited in these cells by incubation with 7773.

As a second test for direct 7773 targeting of Igf2bp1 in cells, the inventors developed an assay based on the requirement of Igf2bp1 to dimerize in order to stably bind RNA (FIG. 8A). Vectors expressing fragments of a split luciferase reporter with a flexible linker were fused to Igf2bp1 in different orientations, and the combination and concentration that gave optimal luminescence was identified by transfection into RKO cells (that express very low levels of Igf2bp1(FIG. 8B). When dimerization of the Igf2bp1-luciferase reporter fusions was inhibited by co-transfection of an Igf2bp1 overexpression plasmid, luminescence was significantly reduced compared to co-transfection with a control plasmid (with no insert; FIG. 8C). This result was consistent with previous reports demonstrating the presence of Igf2bp1 dimers/multimers in RNP complexes and validates the utility of the inventors reporter system for detecting these interactions. Incubation with the 7773 inhibitor significantly reduced luminescence in RKO cells transfected with the luciferase reporter fusions, indicating that the compound was targeting Igf2bp1 in these cells (FIG. 8D). Importantly, 7773 did not affect the activity of wild type luciferase, demonstrating its specificity for Igf2bp1 (FIG. 8E).

### 7773 Inhibits Neoplastic Activity

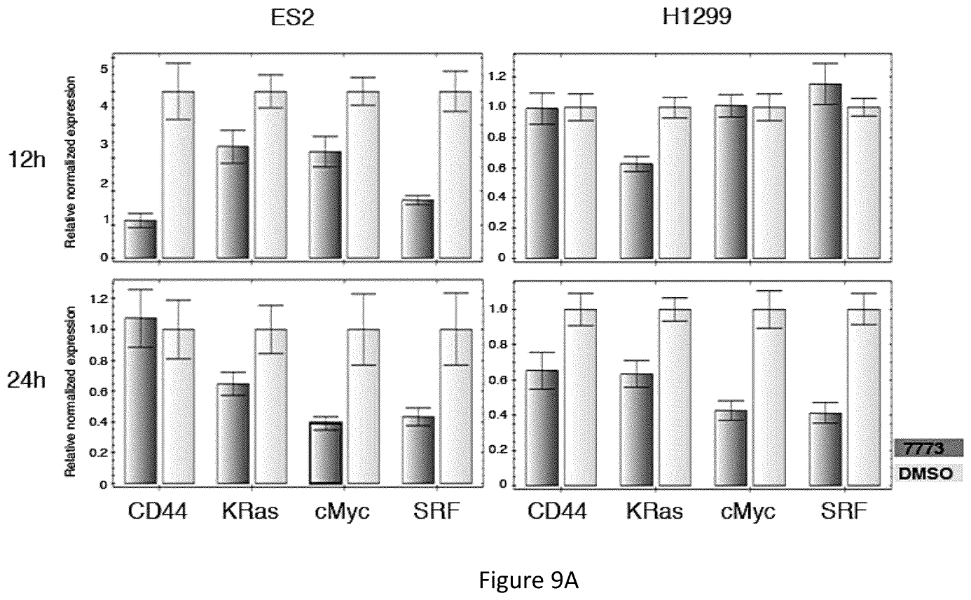

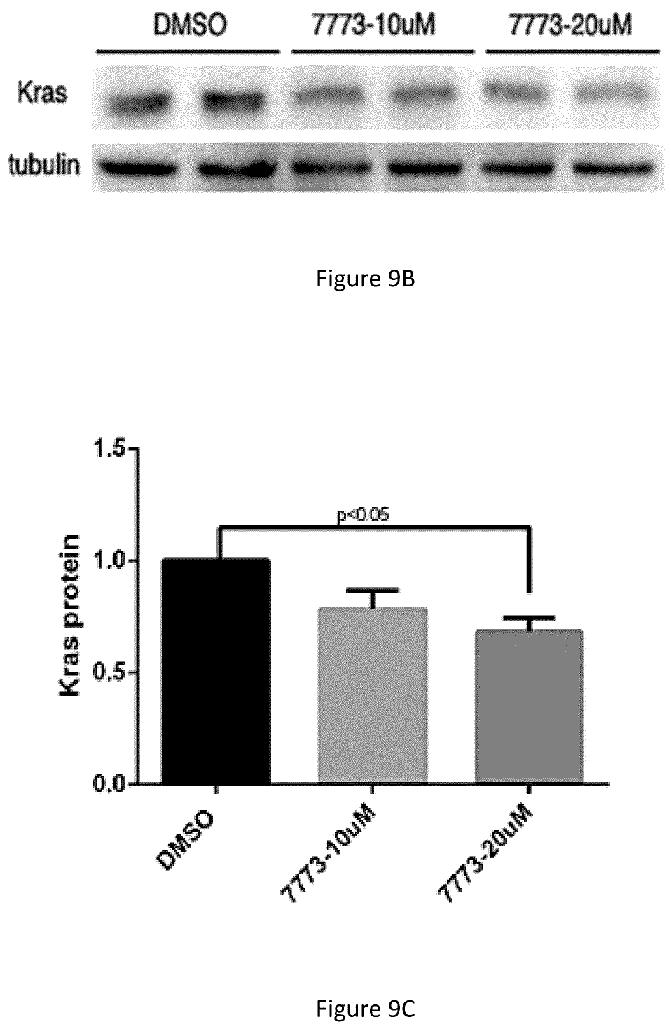

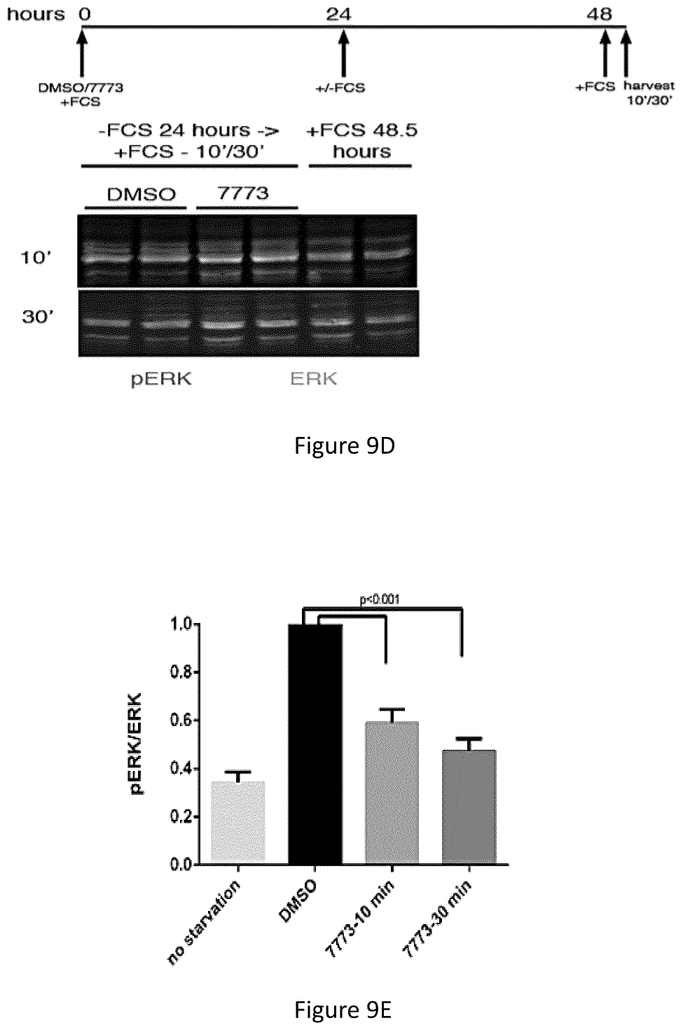

Igf2bp1 has been shown to stabilize a number of its RNA targets through binding to either coding or non-coding sequences in the mRNA. To see whether 7773 inhibition of Igf2bp1 affects target RNA levels, the inventors analyzed steady state levels of several previously identified, cancer-associated RNA targets in ES2 and H1299 cancer cell lines, which express high levels of Igf2bp1, using quantitative PCR (FIG. 9A). Incubation of cells with 7773 for either 12 or 24 hours caused a clear reduction in steady state levels for all the RNAs assayed, although the degree and timing of the reduction was both cell-type and RNA dependent. In the ovarian carcinoma line, ES2, all of the assayed RNAs were reduced after 12 hours, with 3 of the 4 RNAs showing similar or even enhanced reduction after 24 hours. One of the RNAs, CD44, was strongly reduced after 12 hours, but appeared to recover after 24 hours. In the lung adenocarcinoma line, H1299, Kras mRNA was already reduced after 12 hours, with all of the RNAs significantly reduced after 24 hours. Control RNAs, which are not targets of Igf2bp1, were not significantly affected by incubation with 7773 after 24 hours in either ES2 or H1299 cells (FIG. 17).

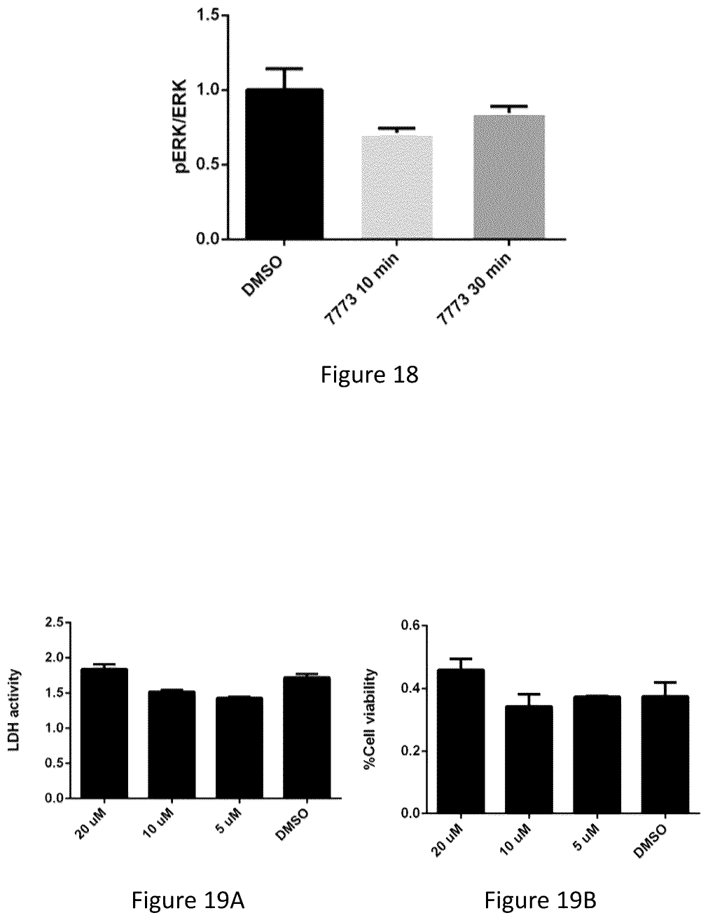

The inventors tested whether the reduction in Kras RNA would lead to a reduction in Kras protein in the H1299 cells. Cells were incubated with DMSO or 7773 for 48 hours and then assayed for expression of Kras protein on a western blot. Indeed, a dose-dependent reduction of Kras protein is observed (FIG. 9B, C). One readout of reduced Kras protein is downstream ERK signaling, which is associated with enhanced wound healing and cell migration. The ratio of phosphorylated ERK (pERK) to total ERK in H1299 cells that were starved and then induced by addition of serum is also reduced by preincubation of the cells with 7773 (FIG. 9D,E). A similar reduction of pERK levels is observed in LKR-M cells expressing human Igf2bp1 (FIG. 18). Taken together, these studies indicate that 7773 targets Igf2bp1 RNA binding in cells, leading to a reduction of Kras RNA, Kras protein, and pERK signaling.

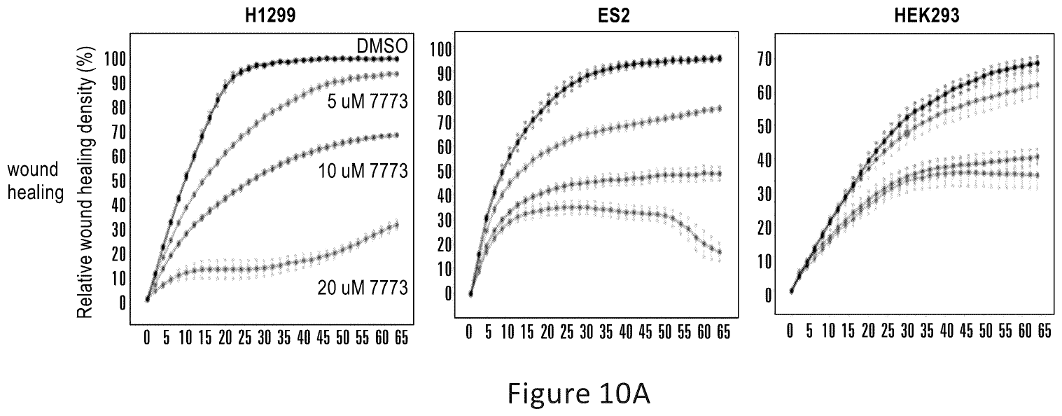

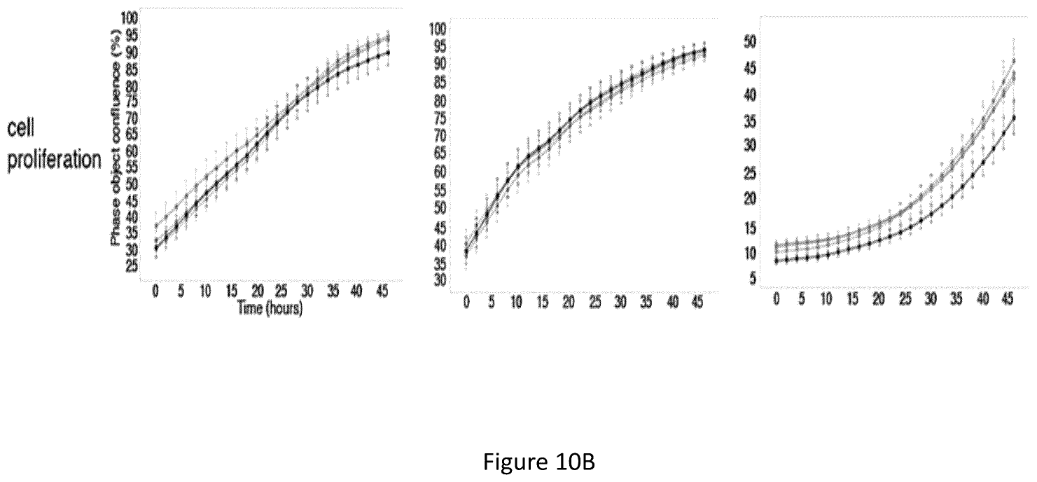

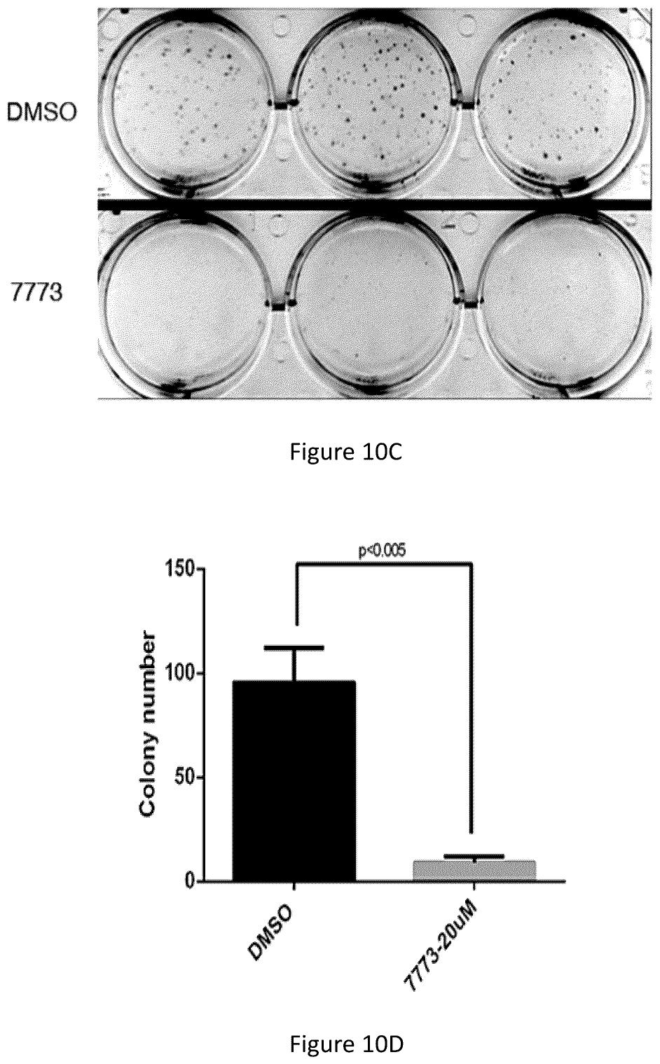

After observing the inhibition of Kras signaling by 7773 on a molecular level, the inventors followed the effects of a 2-3 day exposure to 7773 on cell migration and proliferation in three different human cell lines (H1299, ES2, and HEK293), expressing high levels of Igf2bp1 (FIG. 10A). There was a pronounced repression of wound healing with increasing concentration of 7773; a discernible inhibition was observed at even the lowest concentration, 5 mM, in all of the cell lines. Cell proliferation in all the lines, as assayed by Incucyte, was remarkably unaffected, however, by even the highest concentration of 7773 (20 mM), a concentration that strongly inhibited wound healing in each of the lines (FIG. 10B).

Anchorage-independent growth is a hallmark of lung carcinomas and other neoplastic cells. H1299 cells grow well when cultured in soft agar and form colonies from single cells that are observable by eye after 2 weeks. Incubation of these cells in 20 mM 7773, however, dramatically inhibited their ability to form colonies in soft agar (FIG. 10C, D), despite virtually no effect on cell proliferation or cytotoxicity (FIGS. 19A-B). Taken together, these results demonstrate that the effect of 7773 on lung adenocarcinoma cells is not the result of generalized toxicity but rather specific inhibition of signaling, migration, and growth associated with neoplastic cells.

Therapies directed at inhibiting Igf2bp1 function constitute a potentially powerful approach for fighting cancer, given the correlation between elevated Igf2bp1 expression and poor clinical outcomes, the activation of Igf2bp1 in a wide variety of cancers, and the effectiveness of preventing metastasis when Igf2bp1 activity is reduced. Furthermore, Igf2bp1 is a very attractive target for therapy inasmuch as specific inhibitors would be expected to have minimal side effects: i) Igf2bp1 is expressed at very low levels in normal adult tissues, and ii) adult mice with an inducible whole-mouse knockout of Igf2bp1 in adult animals (utilizing newly generated UBC-Cre^{ERT2}; Igf2bp1^{loxP/loxP} and Rosa26-Cre^{ERT2}; Igf2bp1^{loxP/loxP} mice) are healthy. For these reasons, the inventors undertook a screen to identify small molecule inhibitors of Igf2bp1. Evidence presented here indicates that a hit identified in the screen is a very promising lead molecule. In vitro, 7773 binds Igf2bp1 and inhibits its ability to bind Kras 6 RNA. When incubated with cells, 7773 targets Igf2bp1 and causes a reduction in Kras mRNA and other RNA targets, reduces Kras protein and downstream signaling, and represses cell migration and growth in soft agar. The inventors anticipate that further refinement of this compound will lead to a new class of drugs that can be used in a clinical setting for treating lung adenocarcinomas as well as other neoplasias. The inventors have previously described a role for Igf2bp1 in lung adenocarcinoma progression as well as in cell migration and metastasis. In addition, Igf2bp1 has also been associated with enhancing cancer cell resistance to chemotherapy. The inventors thus envision that an optimized small molecule based on 7773 could be useful in a clinical setting, either as monotherapy directed against cancer progression, cell migration and metastasis formation or perhaps as adjuvant therapy in conjunction with chemotherapeutic agents.

Structural analysis of the binding of 7773 to Igf2bp1 has begun to shed some light on how this molecule functions. NMR analysis indicates that 7773 interferes with the binding of Igf2bp1 to its Kras RNA target by binding to a planar, elongated surface in the KH34 di-domain interface. Interestingly, binding is mainly hydrophobic but is not mediated by the two canonical base-recognition grooves, which are also hydrophobic. As shown previously, KH34 binds bipartite RNA sequences, with each domain contributing to the binding and the RNA spacer between the target sequences spanning the two canonical RNA-binding grooves. It seems possible that 7773 prevents RNA binding by occupying the path the RNA must trace to connect the two grooves. Alternatively, 7773 binding may result in local conformational changes in the residues framing the hydrophobic grooves. The results of the gel shift and MST experiments (FIG. 5) demonstrate that incubation of Igf2bp1 with 7773 shifts complex formation at higher protein concentrations, but also leads to a decrease of signal, indicating a complex effect on binding, possibly related to the use of multiple domains in protein-RNA recognition. Future work, including a higher resolution structural analysis of the interaction, followed by a mutational analysis, will help elucidate the exact mechanism of 7773 inhibition of mRNA binding.

Kras is the most commonly mutated oncogene in cancer and is thought to drive more than 30% of all tumors. It has been a notoriously recalcitrant protein for targeted therapy. Here the inventors were able to obtain a reduction in steady-state Kras mRNA levels by treating lung and ovarian carcinoma cells with 7773 for 24 hours, and this reduction led to reduced Kras protein levels after 48 hours. ERK is a downstream effector of Kras signaling, undergoing phosphorylation and regulating several crucial cell functions, including proliferation and migration. Incubation of lung adenocarcinoma cells with 7773 prior to activation of Kras signaling impairs ERK phosphorylation. Accordingly, treated cells also show a reduction of cell migration that is dependent on the concentration of the compound. Proliferation was strikingly unaffected by any of the concentrations of 7773, indicating that it is not toxic, although the ability of H1299 cells to grow in soft agar was severely impaired by incubation with the compound. Thus, 7773 treatment, by interfering with Igf2bp1 binding to Kras, appears to be a highly selective and effective tool for inhibiting Kras and at least some of its oncogenic downstream effects.

These data are consistent with those reported previously using a mouse lung adenocarcinoma cell line, LKR-M, in which either a human full length (FL-)Igf2bp1 or a dominant negative (DN-)Igf2 bp construct was overexpressed. When these cells were xenografted subcutaneously into syngeneic mice, FL-Igf2bp1 overexpression significantly enhanced, and DN-Igf2 bp overexpression significantly repressed, the number and burden of LKR-M derived lesions in the lungs. Proliferation of these cells, either in vitro or in vivo, was not significantly affected by the expression of either of the constructs. Wound healing, however, was increased by FL-Igf2bp1, and decreased by DN-Igf2 bp due to its ability to inhibit RNA binding of all the Igf2 bp paralogs. 7773 appears to mimic the effects of the DN-Igf2 bp construct, with one notable exception, namely that 7773 does not inhibit wound healing in native LKR-M cells (FIG. 7). Significantly, these cells do not express endogenous Igf2bp1 but rather Igf2bp2, which is not bound by 7773 (FIG. 6). These results thus argue that 7773 targets Igf2bp1 selectively in vivo as well as in vitro. Selective targeting was further validated by the ability of 7773 to inhibit the split-luciferase reporter constructs fused to Igf2bp1, but not control luciferase (FIG. 8). Consistent with these findings is the fact that 7773 satisfies Lipinski's Rule of 5 for drug solubility and cell uptake, having a molecular weight under 500 Daltons and a c Log P of 2.5 (<5).

Several other Igf2bp1 target RNAs (SRF, cMyc, and CD44) are reduced in lung and ovarian carcinoma cells incubated with the compound (FIG. 9). These results are consistent with the fact that Igf2bp1 appears to promote pro-oncogenic phenotypes based on its ability to bind many RNAs associated with neoplastic cells. SRF mRNA is protected from miRNA-mediated degradation via Igf2bp1 binding, and SRF and Igf2bp1 synergize to promote gene expression in cancer. CD44 is a transmembrane glycoprotein whose aberrant expression is associated with invasion and metastasis in various cancers. In HeLa cells, downregulation of Igf2bp1 and 3 causes an almost 3-fold reduction in CD44 mRNA half-life and a loss of invadopodia. cMyc is a transcription factor that regulates growth and proliferation of cells and is associated with a majority of human tumors. Igf2bp1 binds a sequence in the coding region of cMyc mRNA that stabilizes it. Given the large number of Igf2bp1 mRNA targets, it appears likely that many additional RNAs are involved in mediating the effects of Igf2bp1 inhibition; global analysis of RNAs affected by the compound will likely elucidate additional pathways.

A small molecule that inhibited Igf2bp1-cMyc RNA interaction in vitro (termed BTYNB) has been identified using fluorescence polarization. This molecule appears to inhibit cell proliferation, but its mechanism of action, binding site, and specificity have not been characterized. 7773 shows no obvious similarity to BTYNB and has no effect on proliferation, although it does reduce cMyc mRNA levels by 50-60% in different cell lines. Despite the ability of Igf2bp1 to stabilize cMyc and other RNAs when tested in cells in culture, cMyc RNA is not upregulated in tumors induced by Igf2bp1 overexpression in the mammary glands of mice. The importance of the Igf2bp1-cMyc mRNA interaction in tumorigenesis remains to be determined.

By virtue of their ability to bind a wide range of pro-oncogenic mRNAs, Igf2 bp proteins represent an attractive target for directed therapies. Indeed, these proteins have been described as part of an epigenetic switch that occurs during oncogenesis. Igf2 bp proteins are also part of a select group of m6A readers, and methylation of RNAs is upregulated in many tumors, where it is often associated with oncogenic behavior. It is interesting to note that the small circular RNA circNDUFB2 has recently been shown to be inversely correlated with growth and metastasis in NSCLC patients. When expressed at elevated levels, this RNA facilitates ubiquitination and degradation of Igf2bps, and the process is enhanced when the circular RNA is methylated. These results demonstrate the potential benefits of downregulating Igf2 bp proteins by small molecules, and drugs that selectively inhibit these proteins can provide new approaches for fighting tumor progression and metastasis.

### Example 2

### Molecule Optimization

Analogs of compound 7773 were synthetized and screened for their inhibitory activity. Table 4 presents the structure of compounds that were tested and presented inhibitory activity with IC₅₀ between 0.3 μM and 30 μM.

TABLE 4

| Compound No. | Structure |
|---|---|
| 1 | 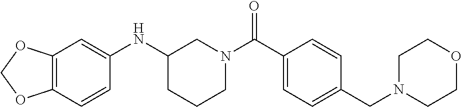 [SMILES: O=C(c1ccc(CN2CCOCC2)cc1)N1CCCC(Nc2ccc3c(c2)OCO3)C1] |
| 2 | 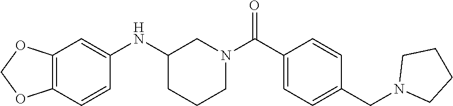 [SMILES: O=C(c1ccc(CN2CCCC2)cc1)N1CCCC(Nc2ccc3c(c2)OCO3)C1] |
| 5 | 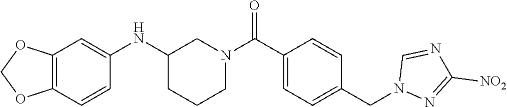 [SMILES: O=C(c1ccc(Cn2cnc([N+](=O)[O-])n2)cc1)N1CCCC(Nc2ccc3c(c2)OCO3)C1] |
| 6 | 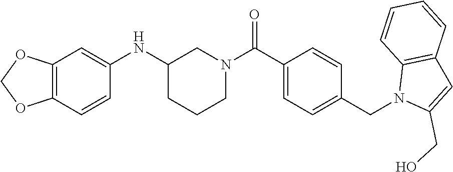 [SMILES: O=C(c1ccc(Cn2c(CO)cc3ccccc32)cc1)N1CCCC(Nc2ccc3c(c2)OCO3)C1] |
| 7 | 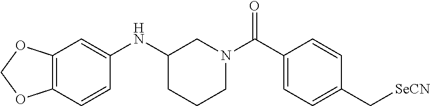 |
| 8 | 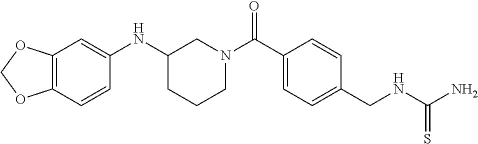 [SMILES: NC(=S)NCc1ccc(C(=O)N2CCCC(Nc3ccc4c(c3)OCO4)C2)cc1] |
| 15 | 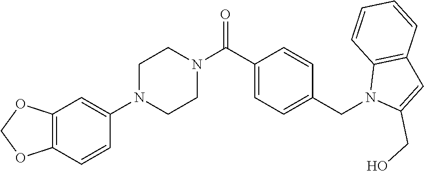 [SMILES: O=C(c1ccc(Cn2c(CO)cc3ccccc32)cc1)N1CCN(c2ccc3c(c2)OCO3)CC1] |
| 17 | 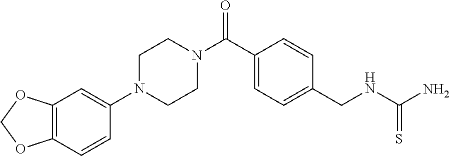 [SMILES: NC(=S)NCc1ccc(C(=O)N2CCN(c3ccc4c(c3)OCO4)CC2)cc1] |
| 22 | 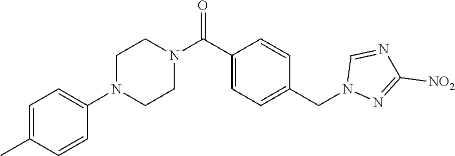 [SMILES: Cc1ccc(N2CCN(C(=O)c3ccc(Cn4cnc([N+](=O)[O-])n4)cc3)CC2)cc1] |
| 24 | 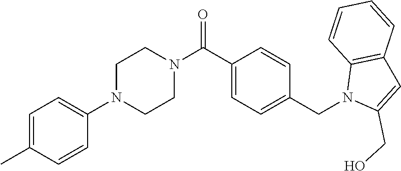 [SMILES: Cc1ccc(N2CCN(C(=O)c3ccc(Cn4c(CO)cc5ccccc54)cc3)CC2)cc1] |

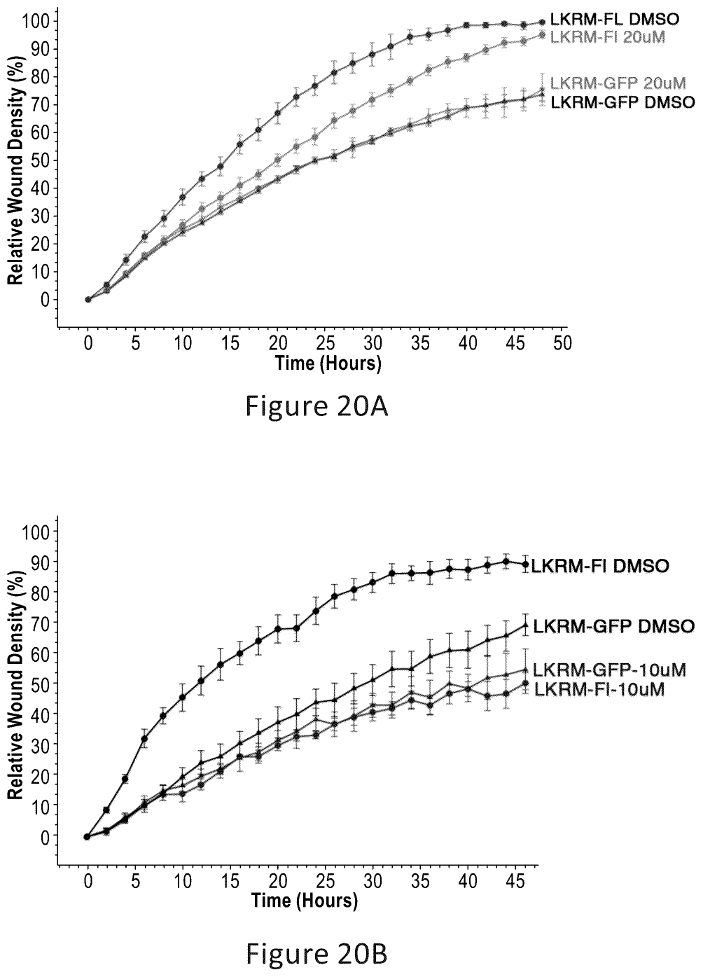

Compound no 15, was over 10-fold more efficient at inhibiting wound healing, in H1299 cells without evidence of any off-target effects, when compared to Compound 7773 (FIGS. 20A-B). Compound 15 also showed inhibition of cell proliferation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A compound or a pharmaceutically acceptable salt thereof, wherein said compound is represented by Formula V: 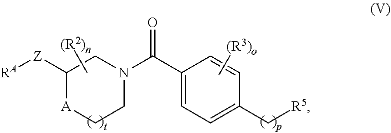
wherein:
R^{A} is absent or selected from 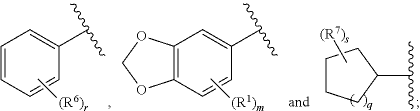
A is selected from CH and N—R, wherein R is 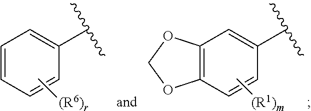
Z is absent, or selected from —C(O)— and —N(R⁸)—, wherein R⁸ is selected from hydrogen and C₁-C₆ alkyl;
m is an integer ranging between 0 and 3;
each n, o and s is independently an integer ranging between 0 and 4;
p is an integer ranging between 0 and 8;
q is an integer ranging between 0 and 2;
r is an integer ranging between 0 and 5;
t is an integer ranging between 1 and 3;
R⁵ is selected from:
a) 3- to 6-membered aliphatic ring, aromatic ring, or heteroaromatic ring having one or two ring heteroatoms independently selected from N, O, or S, wherein the 3- to 6-membered ring is optionally substituted with one or more X groups as allowed by valency;
b) 5- to 10-membered monocyclic or bicyclic aliphatic ring, aromatic ring, or heteroaromatic ring having one, two, three, or four ring heteroatoms selected from N, O, or S, wherein the 5- to 10-membered ring is optionally substituted with one or more X groups as allowed by valency, wherein X is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃ alkyl), (5- to 10-membered monocyclic or bicyclic heteroaryl)(C₀-C₃ alkyl), R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency, wherein Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol;
c) —SeR⁹, wherein R⁹ is selected from hydrogen, cyano, alkyl, cyloalkyl, heterocycle, aryl, or heteroaryl, each of which R⁹ may be optionally substituted with one or more X groups as allowed by valency; and
d) —NH(C═W)NH₂, wherein W is S or Se;
each X, R¹, R², R³, R⁶ and R⁷ represents one or more substituents, each substituent is independently selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency, or wherein two or more of the substituents are interconnected to form a fused aromatic ring, a fused aliphatic ring, or a fused heteroaromatic ring;
each R^{x} and R^{y} is independently selected from hydrogen, C₁-C₆alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;
R^{z} is selected from hydrogen, halo, C₁-C₆ alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency;
Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol; and
if A is N—R, then t is 1 and Z and R^{A} are absent; and if A is CH, Z is NH, p is 1, and R^{A} is 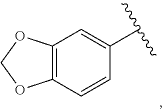 [SMILES: *c1ccc2c(c1)OCO2]
then R5 is devoid of an unsubstituted tetrazole.

2. The compound of claim 1, wherein said compound is represented by Formula VIa: 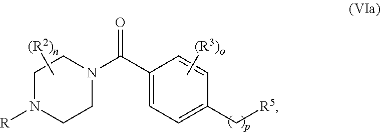
or Formula VIb:

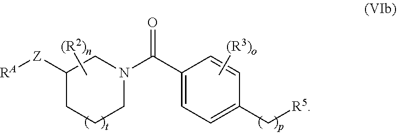

3. The compound of claim 1, wherein r is an integer ranging between 1 and 5.

4. The compound of claim 2, wherein R^{A} is

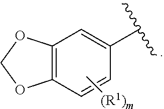

5. The compound of claim 1, wherein R is selected from

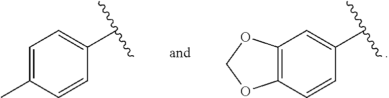

6. The compound of claim 1, wherein R^{A} is

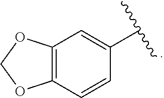 [SMILES: *c1ccc2c(c1)OCO2]

7. The compound of claim 1, wherein said compound is represented by Formula VIIIa: 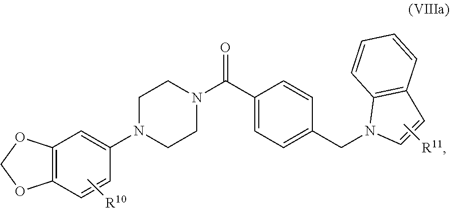
wherein:
each R¹⁰ and R¹¹ is independently selected from hydrogen, halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; and Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

8. The compound of claim 1, wherein said compound is represented by formula VIIIb: 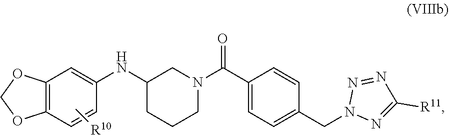
wherein:
each R¹⁰ and R¹¹ is selected from hydrogen, halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl), R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency; Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol; and if R¹⁰ is hydrogen then R¹¹ is not hydrogen.

9. The compound of claim 1, wherein R⁵ is selected from: 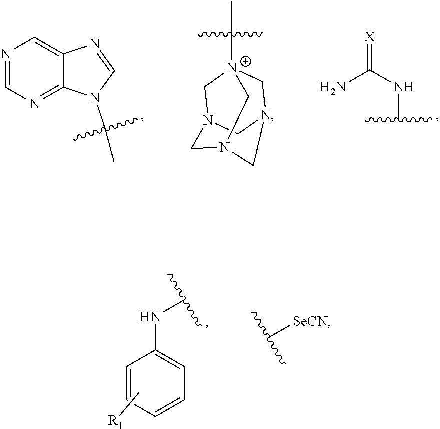
wherein:
X is Se, O or S; and R¹ is selected from H, F, Cl, Br, NO₂, NH₂, CH₃, C₂H₅, CF₃, OH, CN, C₆H₅, CHO, COOH, and any combination thereof.

10. The compound of claim 1, wherein R⁵ is selected from:

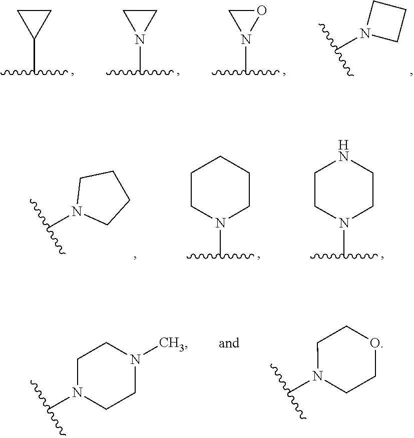

11. The compound of claim 1, wherein R⁵ is selected from:

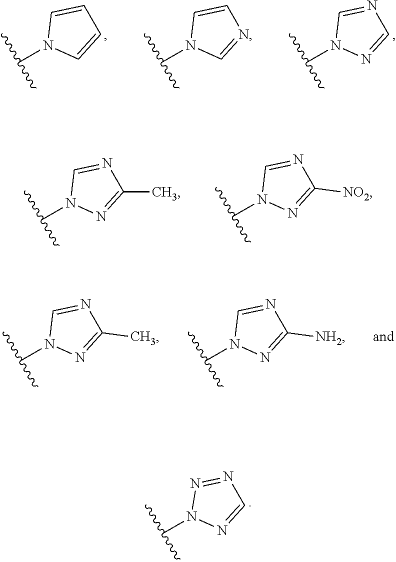

12. The compound of claim 1, wherein R⁵ is selected from: 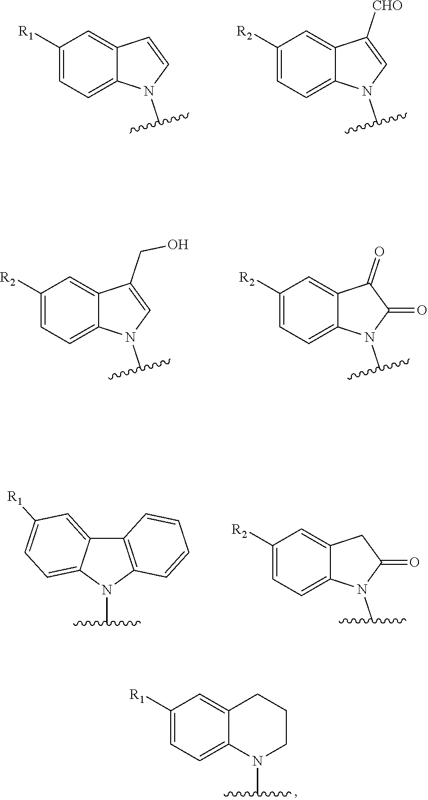
wherein:
each R₁ and R₂ is independently selected from H, F, Cl, Br, NO₂, NH₂, CH₃, C₂H₅, CF₃, OH, CN, C₆H₅, CHO, COOH, and any combination thereof.

13. The compound of claim 1, wherein R⁵ is selected from: 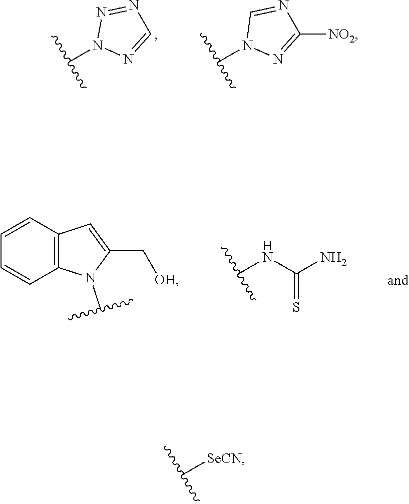
optionally wherein at least one of: Z is —N(R⁸)—; R⁸ is hydrogen; and p is 1.

14. (canceled)

15. (canceled)

16. (canceled)

17. The compound of claim 1, wherein the compound comprises:

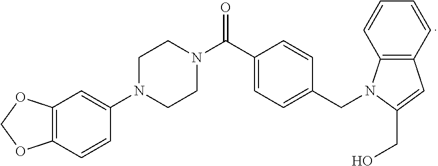 [SMILES: O=C(c1ccc(Cn2c(CO)cc3ccccc32)cc1)N1CCN(c2ccc3c(c2)OCO3)CC1]

18. A pharmaceutical composition comprising a therapeutically effective amount of a compound, a pharmaceutically acceptable salt thereof or both, and a pharmaceutically acceptable carrier or excipient, wherein said compound is represented by Formula V: 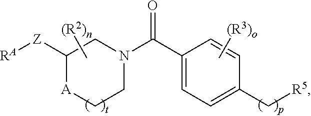
wherein:
R^{A} is absent or selected from 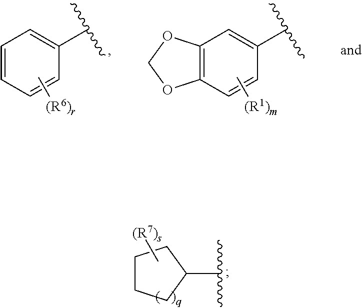
A is selected from CH and N—R, wherein R is 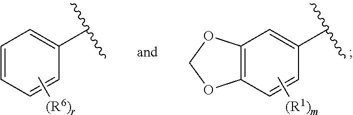
Z is absent, or selected from —C(O)— and —N(R⁸)—, wherein R⁸ is selected from hydrogen and C₁-C₆ alkyl;
m is an integer ranging between 0 and 3;
each n, o and s is independently an integer ranging between 0 and 4;
p is an integer ranging between 0 and 8;
q is an integer ranging between 0 and 2;
r is an integer ranging between 0 and 5;
t is an integer ranging between 1 and 3;
R⁵ is selected from:
a) 3- to 6-membered aliphatic ring, aromatic ring, or heteroaromatic ring having one or two ring heteroatoms independently selected from N, O, or S, wherein the 3- to 6-membered ring is optionally substituted with one or more X groups as allowed by valency;
b) 5- to 10-membered monocyclic or bicyclic aliphatic ring, aromatic ring, or heteroaromatic ring having one, two, three, or four ring heteroatoms selected from N, O, or S, wherein the 5- to 10-membered ring is optionally substituted with one or more X groups as allowed by valency, wherein X is selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl), (3- to 6-membered monocyclic heterocycle)(C₀-C₃ alkyl), (6- to 10-membered monocyclic or bicyclic aryl)(C₀-C₃ alkyl), (5- to 10-membered monocyclic or bicyclic heteroaryl)(C₀-C₃ alkyl), R^{x}O—(C₀-C₃ alkyl)-, RxS—(C₀-C₃ alkyl)-, (RxRyN)—(C₀-C₃ alkyl)-, RzC(O)—O—(C₀-C₃ alkyl)-, RzC(O)—(RxN)—(C₀-C₃ alkyl)-, R^{z}S(O)₂—O—(C₀-C₃ alkyl)-, R^{z}S(O)₂—(R^{x}N)—(C₀-C₃ alkyl)-, RzC(O)—, R^{z}S(O)—, and R^{z}S(O)₂—, each of which may be optionally substituted with one or more Y groups as allowed by valency, wherein Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol;
c) —SeR⁹, wherein R⁹ is selected from hydrogen, cyano, alkyl, 11 ycloalkyl, heterocycle, aryl, or heteroaryl, each of which R⁹ may be optionally substituted with one or more X groups as allowed by valency; and
d) —NH(C═W)NH₂, wherein W is S or Se;
each X, R¹, R², R³, R⁶ and R⁷ represents one or more substituents, each substituent is independently selected from halo, nitro, cyano, azido, C₁-C₆ alkyl, C₁-C₆ alkoxy, C₁-C₆ haloalkyl, C₂-C₆ alkenyl, C₂-C₆ alkynyl, (C₃-C₆ cycloalkyl)(C₀-C₃ alkyl)-, (3- to 6-membered monocyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—, R^{z}S(O)—, and R^{z}S(O)₂—, each of which may be optionally substituted with one or more Y groups as allowed by valency, or wherein two or more of the substituents are interconnected to form a fused aromatic ring, a fused aliphatic ring, or a fused heteroaromatic ring;
each R^{x} and R^{y} is independently selected from hydrogen, C₁-C₆alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;
R^{z} is selected from hydrogen, halo, C₁-C₆ alkyl, C₁-C₆ haloalkyl, C₂-C₆ alkenyl, C₂-C₆ alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and Y is selected from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

19. The pharmaceutical composition of claim 18, wherein the therapeutically effective amount comprises a concentration of said compound within the pharmaceutical composition of between 100 nM and 50 μM.

20. (canceled)

21. (canceled)

22. A method for preventing or treating cancer in a subject, comprising administering to said subject a therapeutically effective amount of the pharmaceutical composition of claim 18, thereby treating or preventing cancer in the subject.

23. The method of claim 22, wherein said subject is a human subject; and wherein said administering comprises an administration route selected from intravenous administration, intraperitoneal administration, subcutaneous administration, or any combination thereof; optionally wherein said cancer comprises any one of a metastatic cancer, a solid tumor, and a liquid tumor.

24. (canceled)

25. (canceled)

26. (canceled)

27. (canceled)

28. (canceled)

29. The method of claim 22, wherein said treating comprises: (i) reducing intracellular expression of at least one protooncogene, (ii) preventing or reducing metastasis, or both (i) and (ii).

30. The method of claim 22, further comprising a step preceding said administering, comprising determining abundance or levels of any one of: IGF2BP1 transcripts or a protein product thereof, IGF2BP1-RNA complexes, or both, in said subject, wherein an increase in any one of said IGF2BP1 transcripts or a protein product thereof, said IGF2BP1-RNA complexes, or both, in said subject compared to a control, is indicative of said subject being suitable for said treating; optionally wherein said determining is in a sample obtained or derived from the subject.

31. (canceled)
